(19) United States
(12) Patent Application Publication
(10) Pub. No.: US 2025/0019770 A1
(43) Pub. Date: Jan. 16, 2025

(54) CIRCULATING TUMOR DNA FRACTION AND USES THEREOF

(71) Applicant: Foundation Medicine, Inc., Boston, MA (US)

(72) Inventors: Geoffrey R. OXNARD, Arlington, MA (US); Hanna TUKACHINSKY, Boston, MA (US); Russell MADISON, Encinitas, CA (US)

(73) Assignee: Foundation Medicine, Inc., Boston, MA (US)

(21) Appl. No.: 18/709,279

(22) PCT Filed: Nov. 11, 2022
(86) PCT No.: PCT/US2022/079736

§ 371 Date: May 10, 2024

Related U.S. Application Data
(60) Provisional application No. 63/305,776, filed on Feb. 2, 2022.
(60) Provisional application No. 63/279,023, filed on Nov. 12, 2021.

(51) Int. Cl. C12Q 1/6886; C12Q 1/6869

(52) U.S. Cl. CPC C12Q 1/6886; C12Q 1/6869; C12Q 2600/118; C12Q 2600/156

(57) ABSTRACT

Disclosed herein are methods of treating an individual having cancer, of treating or identifying an individual having cancer for a treatment, or stratifying individuals having cancer for a treatment based on a tumor shed value determination in a liquid biopsy sample. Also described herein are methods of analyzing a biomarker based on a tumor shed value determination in a liquid biopsy sample.

### CROSS-REFERENCE TO RELATED APPLICATIONS

This application claims the benefit of U.S. Provisional Application Nos. 63/279,023, filed Nov. 12, 2021, and 63/305,776, filed Feb. 2, 2022, each of which is hereby incorporated by reference in its entirety.

### FIELD

Provided herein are methods of selecting a treatment for an individual having cancer, of treating or identifying an individual having cancer for a treatment, or stratifying individuals having cancer for a treatment based on a tumor shed value determination. Also provided herein are methods of analyzing a biomarker based on a tumor shed value determination.

### BACKGROUND

Cancer can be caused by genomic mutations, and cancer cells may accumulate mutations during cancer development and progression. These mutations may be the consequence of intrinsic malfunction of DNA repair, replication, or modification mechanisms, or may be a consequence of exposure to external mutagens. Certain mutations confer growth advantages on cancer cells and are positively selected in the microenvironment of the tissue in which the cancer arises. Detection of these mutations in patient samples using next generation sequencing (NGS) or other genomic analysis techniques can provide valuable insights with respect to diagnosis, prognosis, and treatment of cancer. However, translating the results of genomic studies into routine clinical practice remains expensive, time intensive, and technically challenging.

Liquid biopsy has become a promising tool for applying the results of genomics studies to practical clinical application. One of the challenges of detecting mutations related to cancer in liquid biopsy samples is the low abundance of circulating tumor DNA (ctDNA) shed by cancerous tissue into the bloodstream relative to the total amount of cell-free DNA (cfDNA) present, as well as the often very low allele frequencies of the mutations of interest. Similarly, the low abundance of circulating tumor DNA in a liquid biopsy sample has made predicting response to a treatment regimen challenging.

Thus, there is a need for novel methods that identify an individual for treatment based on determinations of circulating tumor fraction.

### BRIEF SUMMARY OF THE INVENTION

In one aspect, provided herein is a method for identifying an individual having a cancer for treatment with an immuno-oncology (IO) therapy and chemotherapy combination comprising (a) determining a tumor shed value for a liquid biopsy sample obtained from the individual, and (b) identifying the individual for treatment with the IO therapy and chemotherapy combination if the tumor shed value in the liquid biopsy sample is equal to or higher than a reference tumor shed value.

In another aspect, provided herein is a method of treating an individual having a cancer with an immuno-oncology (IO) therapy and chemotherapy combination comprising (a) determining a tumor shed value for a liquid biopsy sample obtained from the individual, and (b) treating the individual with the IO therapy and chemotherapy combination if the tumor shed value in the liquid biopsy sample is equal to or higher than a reference tumor shed value.

In another aspect, provided herein is a method of selecting a treatment for an individual having a cancer, the method comprising determining a tumor shed value for a liquid biopsy sample obtained from the individual, wherein a tumor shed value in the liquid biopsy sample that is equal to or higher than a reference tumor shed value identifies the individual as one who may benefit from treatment with an immuno-oncology (IO) therapy and chemotherapy combination.

In another aspect, provided herein is a method of identifying one or more treatment options for an individual having a cancer, the method comprising: determining a tumor shed value for a liquid biopsy sample obtained from the individual, and generating a report comprising one or more treatment options identified for the individual based at least in part on the tumor shed value for the liquid biopsy sample, wherein a tumor shed value in the liquid biopsy sample that is equal to or higher than a reference tumor shed value identifies the individual as one who may benefit from treatment with an immuno-oncology (IO) therapy and chemotherapy combination.

In another aspect, provided herein is a method of predicting survival of an individual having a cancer, comprising acquiring knowledge of a tumor shed value for a liquid biopsy sample obtained from the individual, wherein the if tumor shed value for the liquid biopsy sample obtained from the individual is equal to or higher than a reference tumor shed value, and wherein responsive to the acquisition of said knowledge, the individual is predicted to have longer survival when treated with an immuno-oncology (IO) therapy and chemotherapy combination, as compared to treatment with an immuno-oncology (IO) therapy without chemotherapy.

In another aspect, provided herein is a method of monitoring, evaluating, or screening an individual having a cancer, comprising acquiring knowledge of a tumor shed value for a liquid biopsy sample obtained from the individual, wherein if the tumor shed value for the liquid biopsy sample obtained from the individual is equal to or higher than a reference tumor shed value, and wherein responsive to the acquisition of said knowledge, the individual is predicted to have longer survival when treated with an immuno-oncology (IO) therapy in combination with chemotherapy, as compared to treatment with an immuno-oncology (IO) therapy without chemotherapy.

In another aspect, provided herein is a method for identifying an individual having a cancer for treatment with an immuno-oncology (IO) therapy comprising (a) determining a tumor shed value for a liquid biopsy sample obtained from the individual, and (b) identifying the individual for treatment with the IO therapy if the tumor shed value in the liquid biopsy sample is lower than a reference tumor shed value.

In another aspect, provided herein is a method of treating an individual having a cancer with an immuno-oncology (IO) therapy comprising (a) determining a tumor shed value for a liquid biopsy sample obtained from the individual, and (b) treating the individual with the IO if the tumor shed value in the liquid biopsy sample is lower than a reference tumor shed value.

In another aspect, provided herein is a method of selecting a treatment for an individual having a cancer, the method comprising determining a tumor shed value for a liquid biopsy sample obtained from the individual, wherein a tumor shed value in the liquid biopsy sample that is lower than a reference tumor shed value identifies the individual as one who may benefit from treatment with an immuno-oncology (IO) therapy.

In another aspect, provided herein is a method of identifying one or more treatment options for an individual having a cancer, the method comprising: determining a tumor shed value for a liquid biopsy sample obtained from the individual, and generating a report comprising one or more treatment options identified for the individual based at least in part on the tumor shed value for the liquid biopsy sample, wherein a tumor shed value in the liquid biopsy sample that is lower than a reference tumor shed value identifies the individual as one who may benefit from treatment with an immuno-oncology (IO) therapy.

In another aspect, provided herein is a method of predicting survival of an individual having a cancer, comprising acquiring knowledge of a tumor shed value for a liquid biopsy sample obtained from the individual, wherein the if tumor shed value for the liquid biopsy sample obtained from the individual is lower than a reference tumor shed value, and wherein responsive to the acquisition of said knowledge, the individual is predicted to have longer survival when treated with an immuno-oncology (IO) therapy, as compared to treatment without immuno-oncology (IO) therapy.

In another aspect, provided herein is a method of monitoring, evaluating, or screening an individual having a cancer, comprising acquiring knowledge of a tumor shed value for a liquid biopsy sample obtained from the individual, wherein if the tumor shed value for the liquid biopsy sample obtained from the individual is lower than a reference tumor shed value, and wherein responsive to the acquisition of said knowledge, the individual is predicted to have longer survival when treated with an immuno-oncology (IO) therapy, as compared to treatment without an immuno-oncology (IO) therapy.

In another aspect, provided herein is a method of stratifying an individual with a cancer for treatment with a therapy comprising determining a tumor shed value for a liquid biopsy sample obtained from the individual, and (a) if the tumor shed value is equal to or higher than a reference tumor shed value, identifying the individual as a candidate for receiving an IO therapy in combination with chemotherapy; or (b) if the tumor shed value is less than the reference tumor shed value, identifying the individual as a candidate for receiving an immuno-oncology (IO) therapy without chemotherapy.

In another aspect, provided herein is a method for identifying an individual having a cancer for treatment with a first therapy and a second therapy comprising (a) determining a tumor shed value for a liquid biopsy sample obtained from the individual, and (b) identifying the individual for treatment with the first therapy and the second therapy if the tumor shed value in the liquid biopsy sample is equal to or greater than a reference tumor shed value.

In another aspect, provided herein is a method of treating an individual having a cancer with a first therapy and a second therapy comprising (a) determining a tumor shed value for a liquid biopsy sample obtained from the individual, and (b) treating the individual with the first therapy and the second therapy if the tumor shed value in the liquid biopsy sample is equal to or greater than a reference tumor shed value.

In another aspect, provided herein is a method of selecting a treatment for an individual having a cancer, the method comprising determining a tumor shed value for a liquid biopsy sample obtained from the individual, wherein a tumor shed value in the liquid biopsy sample that is equal to or greater than a reference tumor shed value identifies the individual as one who may benefit from treatment with a first therapy and a second therapy.

In another aspect, provided herein is a method of identifying one or more treatment options for an individual having a cancer, the method comprising: determining a tumor shed value for a liquid biopsy sample obtained from the individual, and generating a report comprising one or more treatment options identified for the individual based at least in part on the tumor shed value for the liquid biopsy sample, wherein a tumor shed value in the liquid biopsy sample that is equal to or greater than a reference tumor shed value identifies the individual as one who may benefit from treatment with a first therapy and a second therapy.

In another aspect, provided herein is a method of predicting survival of an individual having a cancer, comprising acquiring knowledge of a tumor shed value for a liquid biopsy sample obtained from the individual, wherein the tumor shed value for the liquid biopsy sample obtained from the individual is equal to or greater than a reference tumor shed value, and wherein responsive to the acquisition of said knowledge, the individual is predicted to have longer survival when treated with a first therapy and a second therapy, as compared to treatment with the first therapy without the second therapy.

In another aspect, provided herein is a method of monitoring, evaluating, or screening an individual having a cancer, comprising acquiring knowledge of a tumor shed value for a liquid biopsy sample obtained from the individual, wherein the tumor shed value for the liquid biopsy sample obtained from the individual is equal to or greater than a reference tumor shed value, and wherein responsive to the acquisition of said knowledge, the individual is predicted to have longer survival when treated with a first therapy and a second therapy, as compared to treatment the first therapy without the second therapy.

In another aspect, provided herein is a method of stratifying an individual with a cancer for treatment with a first therapy and a second therapy comprising determining a tumor shed value for a liquid biopsy sample obtained from the individual, and (a) if the tumor shed value is equal to or greater than a reference tumor shed value, identifying the individual as a candidate for receiving a first therapy and a second therapy; or (b) if the tumor shed value is less than a reference tumor shed value, identifying the individual as a candidate for receiving the first therapy without the second therapy.

In some embodiments, the first therapy is an immuno-oncology (IO) therapy. In some embodiments the second therapy is a chemotherapy.

In yet another aspect, provided herein is a method of assessing a biomarker in a liquid biopsy sample from an individual having cancer, the method comprising determining a tumor shed value for the individual, and wherein the tumor shed value is equal to or greater than a reference tumor shed value, further analyzing the biomarker.

In some embodiments, the biomarker is one or more of a tumor mutational burden (TMB) score, a homologous recombination deficiency (HRD) score, or a microsatellite instability (MSI) status. In some embodiments, the TMB score is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb. In some embodiments, the TMB is at least about 5 mutations/Mb. In some embodiments, the TMB score is at least about 10 mutations/Mb. In some embodiments, the TMB score is at least about 12 mutations/Mb. In some embodiments, the TMB score is at least about 16 mutations/Mb. The method of any one of claims 25-29, wherein the TMB score is at least about 20 mutations/Mb. In some embodiments, the TMB score is at least about 30 mutations/Mb. In some embodiments, the TMB score is determined based on between about 100 kb to about 10 Mb. In some embodiments, the TMB score is determined based on between about 0.8 Mb to about 1.1 Mb. In some embodiments, the TMB score is a blood TMB (bTMB) score. In some embodiments, the MSI status is a MSI high or MSI low status. In some embodiments, the MSI status is an MSI stable status. In some embodiments, the HRD score is a HRD-positive score, or a HRD-negative score.

In some embodiments, the biomarker comprises one or more alterations in one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, C11orf30, C17orf39, CALR, CARD11, CASP8, CBFB, CBL, CCND1, CCND2, CCND3, CCNE1, CD22, CD70, CD74, CD79A, CD79B, CD274, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A, KMT2D, KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NSD3, NT5C2, NTRK1 NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK. TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WTI, XPO1, XRCC2, ZNF217, and ZNF703, or any combination thereof. In some embodiments, the biomarker comprises one or more alteration in PIK3CA. In some embodiments, of the one or more alterations comprise a base substitution, an insertion/deletion (indel), a copy number alteration, or a genomic rearrangement.

In some embodiments of any of the methods of the disclosure, the tumor shed value is determined by composite tumor fraction (cTF) or by a tumor fraction estimator (TFE) process.

In some embodiments, the tumor shed value is determined by cTF using a method comprising: receiving a plurality of values, each value indicative of an allele fraction at a corresponding locus within a subgenomic interval in the sample; determining a certainty metric value indicative of a dispersion of the plurality of values; determining a first estimate of the tumor fraction of the sample, the first estimate based on the certainty metric value for the sample and a predetermined relationship between one or more stored certainty metric values and one or more stored tumor fraction values; determining whether a value associated with the first estimate is greater than a first threshold, wherein if the value associated with the first estimate is greater than the first threshold, the first estimate determined as the tumor fraction of the sample; and if the value associated with the first estimate is less than or equal to the first threshold, determining a second estimate of the tumor fraction of the sample based on an allele frequency determination, wherein the second estimate is determined as the tumor fraction of the sample. In some embodiments, the tumor fraction is a value indicative of a ratio of circulating tumor DNA (ctDNA) to total cell-free DNA (cfDNA) in the sample. In some embodiments, the first threshold is indicative of a minimum detectable quantity for the tumor fraction of the sample. In some embodiments, determining whether the value associated with the first estimate of the tumor fraction of the sample is greater than the first threshold comprises determining whether the first estimate is greater than a defined tumor fraction threshold. In some embodiments, determining whether the value associated with the first estimate of the tumor fraction of the sample is greater than a first threshold comprises determining whether a statistical lower bound associated with the first estimate is greater than 0. In some embodiments, determining the second estimate of the tumor fraction of the sample based on the allele frequency determination comprises: determining whether a quality metric for the plurality of values is greater than a second threshold; based on a determination that the quality metric for the plurality of values is greater than the second threshold, determining the second estimate for the tumor fraction of the sample based on a first determination of somatic allele frequency, and based on a determination that the quality metric for the plurality of values is less than or equal to the second threshold, determining the second estimate for the tumor fraction of the sample based on a second determination of somatic allele frequency. In some embodiments, the quality metric for the plurality of values is indicative of an average sequence coverage for the sample, an allele coverage at each loci corresponding to the plurality of values, a degree of nucleic acid contamination in the sample, a number of single nucleotide polymorphism (SNP) loci within the loci corresponding to the plurality of values, or any combination thereof. In some embodiments, the quality metric for the plurality of values is indicative of a minimum average sequence coverage for the sample, a minimum allele coverage at each of the loci corresponding to the plurality of values, a maximum degree of nucleic acid contamination in the sample, a minimum number of single nucleotide polymorphism (SNP) loci within the loci corresponding to the plurality of values, or any combination thereof. In some embodiments, the second threshold comprises a specified lower limit of the quality metric. In some embodiments, the first determination of somatic allele frequency comprises a determination of variant allele frequencies associated with the plurality of values after excluding variant alleles that are present at an allele frequency greater than an upper bound for the first estimate of the tumor fraction of the sample, and the second estimate of the tumor fraction of the sample is set equal to a maximum variant allele frequency detected. In some embodiments, the second determination of somatic allele frequency comprises a determination of variant allele frequencies for all variant alleles associated with the plurality of values, and the second estimate of the tumor fraction of the sample is set equal to a maximum variant allele frequency detected. In some embodiments, the first determination of somatic allele frequency and the second determination of somatic allele frequency further comprise removing variant allele frequencies from the determination that correspond to germline variants, clonal hematopoiesis of indeterminate potential (CHIP) variants, and sequencing artifact variants, prior to determining the second estimate of the tumor fraction of the sample. In some embodiments, the first determination of somatic allele frequency and the second determination of somatic allele frequency further comprise using a variant allele frequency for a rearrangement as the second estimate of the tumor fraction of the sample if rearrangements are detected in the sample. In some embodiments, the first determination of somatic allele frequency and the second determination of somatic allele frequency further comprise removing variant allele frequencies that correspond to variants of unknown significance prior to determining the second estimate of the tumor fraction of the sample. In some embodiments, each value within the plurality of values is an allele fraction. In some embodiments, each value within the plurality of values comprises a ratio of the difference in abundance between a maternal allele and a paternal allele relative to abundance of the maternal allele or the paternal allele at the corresponding locus. In some embodiments, the certainty metric value for the sample is indicative of a deviation of each of the plurality of values from a corresponding expected value. In some embodiments, the corresponding expected value is a locus-specific expected value. In some embodiments, the certainty metric for the sample is a root mean squared deviation of the plurality of values from their corresponding expected values. In some embodiments, the corresponding expected value is an expected allele frequency for a non-tumorous sample. In some embodiments, each value within the plurality of values is an allele fraction, and the expected value is about 0.5. In some embodiments, each value within the plurality of values is a ratio of the difference in abundance between a maternal allele and a paternal allele, relative to an abundance of the maternal allele or the paternal allele at the corresponding locus, and the expected value comprises the expected ratio of the difference in abundance between a maternal allele and a paternal allele, relative to an abundance of the maternal allele or the paternal allele, wherein the expected value is the expected ratio for a non-tumorous sample. In some embodiments, the corresponding expected value is about 0. In some embodiments, the plurality of values comprises a plurality of allele coverages. In some embodiments, the method further comprising determining a probability distribution function for the plurality of values; wherein the certainty metric value for the sample is determined using the probability distribution function. In some embodiments, the certainty metric value for the sample is an entropy of the probability distribution function. In some embodiments, the corresponding loci comprise one or more loci having a different maternal allele and paternal allele. In some embodiments, the corresponding loci consist of loci having a different maternal allele and paternal allele. In some embodiments, the corresponding loci comprise one or more loci having the same maternal allele and paternal allele.

In some embodiments, the tumor shed value is determined by a TFE process using a method comprising: receiving a plurality of values, each value indicative of an allele fraction at a corresponding locus within a subgenomic interval in the sample; determining a certainty metric value indicative of a dispersion of the plurality of values; and determining an estimate of the tumor fraction of the sample based on the certainty metric value for the sample and a predetermined relationship between one or more stored certainty metric values and one or more stored tumor fraction values, wherein the estimate is determined as the tumor fraction of the sample. In some embodiments, the tumor fraction is a value indicative of a ratio of ctDNA to total cfDNA in the sample. In some embodiments, each value within the plurality of values is an allele fraction. In some embodiments, each value within the plurality of values comprises a ratio of the difference in abundance between a maternal allele and a paternal allele relative to abundance of the maternal allele or the paternal allele at the corresponding locus. In some embodiments, the certainty metric value for the sample is indicative of a deviation of each of the plurality of values from a corresponding expected value. In some embodiments, the plurality of values comprises a plurality of allele coverages. In some embodiments, the method further comprising determining a probability distribution function for the plurality of values; wherein the certainty metric value for the sample is determined using the probability distribution function. In some embodiments, the certainty metric value for the sample is an entropy of the probability distribution function. In some embodiments, the corresponding loci comprise one or more loci having a different maternal allele and paternal allele. In some embodiments, the corresponding loci consist of loci having a different maternal allele and paternal allele. In some embodiments, the corresponding loci comprise one or more loci having the same maternal allele and paternal allele.

In some embodiments of any of the methods of the disclosure, the reference tumor shed value is between 0.5% to 10.0%. In some embodiments, the reference tumor shed value is 0.5%. In some embodiments, the reference tumor shed value is 1.0%. In some embodiments, the reference tumor shed value is 2.0%. In some embodiments, the IO therapy comprises a single IO agent or multiple IO agents.

In some embodiments of any of the methods of the disclosure, the IO therapy comprises an immune checkpoint inhibitor. In some embodiments, the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In some embodiments, the immune checkpoint inhibitor is a PD-L1-inhibitor. In some embodiments, the immune checkpoint inhibitor comprises one or more of atezolizumab, avelumab, or durvalumab. In some embodiments, the immune checkpoint inhibitor is a CTLA-4 inhibitor. In some embodiments, the CTLA-4 inhibitor comprises ipilimumab. In some embodiments, the nucleic acid comprises a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments of any of the methods of the disclosure, the chemotherapy comprises a single chemotherapeutic agent or multiple therapeutic agents. In some embodiments, the chemotherapy comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, farnesyl-protein transferase inhibitor, transplatinum, or any combination thereof.

In some embodiments of any of the methods of the disclosure, the survival is progression-free survival (PFS). In some embodiments of any of the methods of the disclosure, the survival is overall survival (OS).

In some embodiments of any of the methods of the disclosure, the method further comprising treating the individual with the IO therapy in combination with chemotherapy. In some embodiments, the IO therapy and the chemotherapy are administered concurrently or sequentially.

In some embodiments of any of the methods of the disclosure, the method further comprising treating the individual with the IO therapy.

In some embodiments of any of the methods of the disclosure, the method further comprising treating the individual with a TMB-targeted therapy. In some embodiments, the TMB-targeted therapy comprises an immune checkpoint inhibitor. In some embodiments, the immune checkpoint inhibitor is an anti-PD1 therapy or an anti-PD-L1 therapy. In some embodiments, the anti-PD-1 therapy comprises one or more of nivolumab, pembrolizumab, cemiplimab, or dostarlimab. In some embodiments, the anti-PD-L1 therapy comprises one or more of atezolizumab, avelumab, or durvalumab.

In some embodiments of any of the methods of the disclosure, the method further comprising treating the individual with a MSI high status an MSI-high-targeted therapy. In some embodiments, the MSI-high-targeted therapy comprises an immune checkpoint inhibitor. In some embodiments, the immune checkpoint inhibitor is an anti-PD1 therapy, an anti-PD-L1 therapy, or an anti-CTLA-4 therapy. In some embodiments, the anti-PD-1 therapy comprises one or more of nivolumab, pembrolizumab, cemiplimab, or dostarlimab. In some embodiments, the anti-PD-L1 therapy comprises one or more of atezolizumab, avelumab, or durvalumab. In some embodiments, the anti-CTLA-4 therapy comprises ipilimumab.

In some embodiments of any of the methods of the disclosure, the method further comprising treating the individual having a HRD-positive score with an HRD-positive targeted therapy. In some embodiments, the HRD-positive targeted therapy is selected from the group consisting of a platinum-based drug and a PARP inhibitor, or any combination thereof. In some embodiments, the PARP inhibitor is olaparib, niraparib, or rucaparib.

In some embodiments of any of the methods of the disclosure, the method further comprising treating the individual with an additional anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some embodiments of any of the methods of the disclosure, the liquid biopsy sample comprises blood, plasma, serum, cerebrospinal fluid, sputum, stool, urine, or saliva. In some embodiments, the liquid biopsy is blood, plasma, or serum. In some embodiments, the liquid biopsy sample comprises mRNA, DNA, circulating tumor DNA (ctDNA), cell-free DNA, or cell-free RNA from the cancer.

In some embodiments of any of the methods of the disclosure, the tumor shed value is determined by sequencing. In some embodiments, the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique. In some embodiments, the sequencing comprises: providing a plurality of nucleic acid molecules obtained from the sample, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; capturing nucleic acid molecules from the amplified nucleic acid molecules, wherein the captured nucleic acid molecules are captured from the amplified nucleic acid molecules by hybridization to one or more bait molecules; sequencing, by a sequencer, the captured nucleic acid molecules to obtain a plurality of sequence reads corresponding to one or more genomic loci within a subgenomic interval in the sample. In some embodiments, the adapters comprise one or more of amplification primer sequences, flow cell adapter hybridization sequences, unique molecular identifier sequences, substrate adapter sequences, or sample index sequences. In some embodiments, amplifying nucleic acid molecules comprises performing a polymerase chain reaction (PCR) technique, a non-PCR amplification technique, or an isothermal amplification technique. In some embodiments, the one or more bait molecules comprise one or more nucleic acid molecules, each comprising a region that is complementary to a region of a captured nucleic acid molecule. In some embodiments, the one or more bait molecules each comprise a capture moiety. In some embodiments, the capture moiety is biotin.

In some embodiments of any of the methods of the disclosure, the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the cancer is a NSCLC, colorectal cancer, cholangiocarcinoma, breast cancer, stomach cancer, melanoma, pancreatic cancer, prostate cancer, ovarian cancer, esophageal cancer, or a cancer of unknown primary.

In some embodiments of any of the methods of the disclosure, the individual is a human. In some embodiments, the individual has previously been treated with an anti-cancer therapy. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended as a definition of the limits of a particular example. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIGS. 1A-1C show the distribution of positive percent agreement (PPA) from simulation data between paired non-small cell lung cancer (NSCLC) tissue and liquid biopsy for companion diagnostic variants stratified by tumor shed. A 1% cut-off was used for tumor shed stratification. Median PPA values are marked by a dotted lines. FIG. 1A shows the distribution of PPA values for samples with ≥1% tumor shed based on maximum allele fraction (MAF). The median PPA value for samples stratified by MAF as having a ≥1% tumor shed was 67%. FIG. 1B shows the distribution of PPA values for samples with ≥1% tumor shed based on composite tumor fraction (cTF). The median PPA value for samples stratified by cTF as having ≥1% tumor shed was 75%. FIG. 1C shows the distribution of PPA values for samples with ≥1% tumor shed based on cTF v2. The median PPA value for samples stratified by cTF v2 as having ≥1% tumor shed was 89%.

FIGS. 2A-2C show the distribution of negative predicted value (NPV) from simulation data between paired NSCLC tissue and liquid biopsy for companion diagnostic variants stratified by tumor shed. A 1% NPV cut-off was used for tumor shed stratification. Median NPV values are marked by a dotted lines. FIG. 2A shows the distribution of NPV values for samples with ≥1% tumor shed based on MAF. The median NPV value for samples stratified by MAF as having a ≥1% tumor shed was 72%. FIG. 2B shows the distribution of NPV values for samples with ≥1% tumor shed based on cTF. The median NPV value for samples stratified by cTF as having a ≥1% tumor shed was 79%. FIG. 2C shows the distribution of NPV values for samples with ≥1% tumor shed based on cTF v2. The median NPV value for samples stratified by cTF v2 as having a 1% tumor shed was 90%.

FIGS. 3A-3C show the distribution of PPA from simulation data between paired NSCLC tissue and liquid biopsy for companion diagnostic variants stratified by tumor shed. A 1% PPA cut-off was used for tumor shed stratification. Median PPA values are marked by a dotted lines. FIG. 3A shows the distribution of PPA values for samples with <1% tumor shed based on MAF. The median PPA value for samples stratified by MAF as having with <1% tumor shed was 41%. FIG. 3B shows the distribution of PPA values for samples with <1% tumor shed based on cTF. The median PPA value for samples stratified by cTF as having with <1% tumor shed was 38%. FIG. 3C shows the distribution of PPA values for samples with <1% tumor shed based on cTF v2. The median PPA value for stratified by cTF v2 as having with <1% tumor shed was 38%.

FIG. 4 shows a receiver operator characteristic (ROC) curve for logistic regression models using tumor shed stratification by MAF, cTF or cTF v2 to predict the presence of tumor in a liquid biopsy. Tumor shed was defined as the detection of companion diagnostic variants in liquid biopsy samples paired to companion diagnostic-positive tissue specimens.

FIGS. 5A-5D show the overall survival (OS) and time to next therapy (TTNT) of NSCLC patients treated with immune-oncology (IO) monotherapy stratified by tumor shed. Patients were stratified based on a cut-off of 1% for either cTF or MAF. The median, 95% lower confidence limit (0.95LCL) and 95% upper confidence level (0.95ULC) are indicated for tumors with ≥1% (1%+) or <1% tumor shed. OS was adjusted for delayed entry. FIG. 5A shows the OS of NSCLC patients stratified by MAF. FIG. 5B shows the TTNT of NSCLC patients stratified by MAF. FIG. 5C shows the OS of NSCLC patients stratified by cTF. FIG. 5D shows the TTNT of NSCLC patients stratified by cTF.

FIGS. 6A-6D show the OS and real-world progression-free survival (rwPFS) of NSCLC patients treated with IO monotherapy, alone or in combination with chemotherapy. Patients were stratified based on a cut-off of 1% for cTF. OS was adjusted for delayed entry. FIG. 6A shows the OS for patients stratified by tumor shed and therapy class. The bottom panel represents the number of patients at risk for each time period of the treatment course. FIG. 6B shows the hazard ratio for the interaction of cTF<1% status and therapy class (3^{rd} line of forest plot) in OS. Patients with <1% treated with a combination of IO therapy and chemotherapy are used as the reference group. Patients with <1% for therapy specific hazard ratios (1^{st} and 2^{nd} line of forest plot). FIG. 6C shows the rwPFS for patients stratified by tumor shed and therapy class. The bottom panel represents the number of patients at risk for each time period of the treatment course. FIG. 6D shows the hazard ratio for the interaction of cTF<1% status and therapy class (3^{rd} line of forest plot) in rwPFS. Patients with <1% treated with a combination of IO therapy and chemotherapy are used as the reference group. Patients with <1% for therapy specific hazard ratios (1^{st} and 2^{nd} line of forest plot). As used in the figure: IO, IO monotherapy; chemIO, IO therapy in combination with chemotherapy; and 95% CI, 95% confidence interval.

FIGS. 7A-7B show the association of tumor shed with biomarker variants in paired cancer tissue and liquid biopsy. FIG. 7A shows the detection of PIK3CA variants in paired tissue and liquid biopsy samples from 206 patients with breast cancer at patient level (left panel) and variant level (right panel). The PPA at patient-level was calculated using the tissue biopsy results as the standard. The PPA was 77% (51/66) and 75% (59/79) at the patient- and variant-level, respectively. FIG. 7B shows the effect of the tumor shed on PIK3CA patient level PPA (solid line) and fraction of cohort (dotted line). Tumor shed was determined by cTF.

FIGS. 8A-8B show the detection of tumor mutation burden (TMB) between paired tissue and liquid biopsy samples stratified by tumor shed. FIG. 8A shows the concordance between liquid biopsy bTMB and tissue TMB in paired samples from 597 patients. The pairs are grouped according to tumor shed. The samples are stratified based on tumor fraction ≥10% (N=270; left panel), ≥1% but lower than 10% (N=277; middle panel), and <1%, (N=150; right panel), the latter including samples with no detectable tumor shed. Tumor shed was calculated by cTF. Spearman's correlation coefficient (ρ) and Lin's Concordance Correlation Coefficient (CCC) were calculated for each group. FIG. 8B shows the distribution of cancer types in different tumor shed cohorts. As used in the figure: TF, tumor fraction; ctDNA shed, circulating tumor DNA shed; bTMB, blood TMB; tTMB, tissue TMB; NSCLC, non-small cell lung cancer; CRC, colorectal cancer; CUP, cancer of unknown primary; and Cholangio, cholangiocarcinoma.

FIG. 9 shows the distribution of tumor shed for liquid biopsy samples harboring complex biomarkers. The plot shows the distribution of tumor shed for all samples analyzed (N=16,381), as well as subgroups of samples having a blood TMB (bTMB)<10 mutations/Mb (N=14,302), bTMB≥10 mutations/Mb (N=2,079), or a microsatellite instability-high (MSI-H) status (N=125). The estimated tumor fraction was calculated by cTF.

FIGS. 10A-10D show the detection of homologous recombination repair deficiency (HRD) scores between paired tissue and liquid biopsy samples. FIG. 10A shows the HRD-positive score detection rates in tissue samples and liquid biopsy samples with cTF>10% by cancer type. FIG. 10B shows a comparison between the frequency of HRD-positive tissue samples and HRD-positive liquid biopsy samples with cTF>10%. FIG. 10C shows a comparison of the HRD-positive fraction between tissue samples and liquid biopsy samples with cTF>10% (triangles), cTF 1-10% (squares), and cTF<1% (circles). FIG. 10D shows the detection rates of BRCA deletions in HRD-positive tissue samples and liquid biopsy samples with cTF>10% from prostate and breast cancer patients. As used in the figures: HRD+, HRD-positive; CUP, unknown primary carcinoma; NSCLC, and non-small cell lung cancer.

### DETAILED DESCRIPTION

Provided herein are methods of identifying an individual having cancer for treatment with an IO therapy, or an IO therapy and chemotherapy combination. In some embodiments, the method comprises (a) determining a tumor shed value for a liquid biopsy sample obtained from the individual, and (b) identifying the individual for treatment with the IO therapy, or the IO therapy and chemotherapy combination. In some embodiments, the method comprises identifying the individual for the IO therapy and chemotherapy combination if the tumor shed value in the liquid biopsy sample is equal to or higher than a reference tumor shed value. In some embodiments, the method comprises identifying the individual for treatment with the IO therapy if the tumor shed value in the liquid biopsy sample is lower than a reference tumor shed value.

Also provided herein are methods of treating an individual having a cancer with an IO therapy, or an IO therapy and chemotherapy combination. In some embodiments, the method comprises (a) determining a tumor shed value for a liquid biopsy sample obtained from the individual, and (b) treating the individual with the IO therapy or the IO therapy and chemotherapy combination. In some embodiments, the method comprises treating the individual with the IO if the tumor shed value in the liquid biopsy sample is lower than a reference tumor shed value. In some embodiments, the method comprises treating the individual with the IO therapy and chemotherapy combination if the tumor shed value in the liquid biopsy sample is equal to or higher than a reference tumor shed value.

Also provided herein are methods of selecting a treatment for an individual having a cancer. In some embodiments, the method comprises determining a tumor shed value for a liquid biopsy sample obtained from the individual. In some embodiments, a tumor shed value in the liquid biopsy sample that is equal to or higher than a reference tumor shed value identifies the individual as one who may benefit from treatment with an IO therapy and chemotherapy combination. In other embodiments, a tumor shed value in the liquid biopsy sample that is lower than a reference tumor shed value identifies the individual as one who may benefit from treatment with an IO therapy.

A method of identifying one or more treatment options for an individual having a cancer, the method comprising: (a) determining a tumor shed value for a liquid biopsy sample obtained from the individual, and (b) generating a report comprising one or more treatment options identified for the individual based at least in part on the tumor shed value for the liquid biopsy sample. In some embodiments, a tumor shed value in the liquid biopsy sample that is equal to or higher than a reference tumor shed value identifies the individual as one who may benefit from treatment with an IO therapy and chemotherapy combination. In some embodiments, a tumor shed value in the liquid biopsy sample that is lower than a reference tumor shed value identifies the individual as one who may benefit from treatment with an IO therapy.

Also provided herein are methods of predicting survival of an individual having a cancer, comprising acquiring knowledge of a tumor shed value for a liquid biopsy sample obtained from the individual. In some embodiments, responsive to the acquisition of knowledge that the tumor shed value for the liquid biopsy sample obtained from the individual is equal to or higher than a reference tumor shed value, the individual is predicted to have longer survival when treated with an IO therapy and chemotherapy combination, as compared to treatment with an IO therapy without chemotherapy. In some embodiments, responsive to the acquisition of knowledge that the tumor shed value for the liquid biopsy sample obtained from the individual is lower than a reference tumor shed value, the individual is predicted to have longer survival when treated with an IO therapy, as compared to treatment without IO therapy. In some embodiments, the survival is the overall survival (OS). In some embodiments, the survival is the progression-free survival (PFS).

Also provided herein are methods of monitoring, evaluating, or screening an individual having a cancer, comprising acquiring knowledge of a tumor shed value for a liquid biopsy sample obtained from the individual. In some embodiments, responsive to the knowledge that the tumor shed value for the liquid biopsy sample obtained from the individual is equal to or higher than a reference tumor shed value, and wherein responsive to the acquisition of said knowledge, the individual is predicted to have longer survival when treated with an immuno-oncology (IO) therapy in combination with chemotherapy, as compared to treatment with an immuno-oncology (IO) therapy without chemotherapy. In some embodiments, responsive to the knowledge that the tumor shed value for the liquid biopsy sample obtained from the individual is lower than a reference tumor shed value, the individual is predicted to have longer survival when treated with an immuno-oncology (IO) therapy, as compared to treatment without an immuno-oncology (IO) therapy. In some embodiments, the survival is the OS. In some embodiments, the survival is the PFS.

Also provided herein are methods of stratifying an individual with a cancer for treatment with a therapy comprising determining a tumor shed value for a liquid biopsy sample obtained from the individual. In some embodiments, if the tumor shed value is equal to or higher than a reference tumor shed value, the method comprises identifying the individual as a candidate for receiving an IO therapy in combination with chemotherapy. In some embodiments, if the tumor shed value is less than the reference tumor shed value, the method comprises identifying the individual as a candidate for receiving an immuno-oncology (IO) therapy without chemotherapy.

Also provided herein are methods for identifying an individual having a cancer for treatment with a first therapy and a second therapy comprising (a) determining a tumor shed value for a liquid biopsy sample obtained from the individual, and (b) identifying the individual for treatment with the first therapy and the second therapy if the tumor shed value in the liquid biopsy sample is equal to or greater than a reference tumor shed value.

Also provided herein are methods of treating an individual having a cancer with a first therapy and a second therapy comprising (a) determining a tumor shed value for a liquid biopsy sample obtained from the individual, and (b) treating the individual with the first therapy and the second therapy if the tumor shed value in the liquid biopsy sample is equal to or greater than a reference tumor shed value. In some embodiments, the first therapy is an IO therapy. In some embodiments, the second therapy is a chemotherapy.

Also provided herein are methods of selecting a treatment for an individual having a cancer, the method comprising determining a tumor shed value for a liquid biopsy sample obtained from the individual, wherein a tumor shed value in the liquid biopsy sample that is equal to or greater than a reference tumor shed value identifies the individual as one who may benefit from treatment with a first therapy and a second therapy. In some embodiments, the first therapy is an IO therapy. In some embodiments, the second therapy is a chemotherapy.

Also provided herein are methods of identifying one or more treatment options for an individual having a cancer, the method comprising: determining a tumor shed value for a liquid biopsy sample obtained from the individual, and generating a report comprising one or more treatment options identified for the individual based at least in part on the tumor shed value for the liquid biopsy sample. In some embodiments, a tumor shed value in the liquid biopsy sample that is equal to or greater than a reference tumor shed value identifies the individual as one who may benefit from treatment with a first therapy and a second therapy. In some embodiments, the first therapy is an IO therapy. In some embodiments, the second therapy is a chemotherapy.

Also provided herein are methods of predicting survival of an individual having a cancer, comprising acquiring knowledge of a tumor shed value for a liquid biopsy sample obtained from the individual. In some embodiments, the tumor shed value for the liquid biopsy sample obtained from the individual is equal to or greater than a reference tumor shed value. In some embodiments, responsive to the knowledge that the tumor shed value for the liquid biopsy sample obtained from the individual is equal to or greater than a reference tumor shed value, the individual is predicted to have longer survival when treated with a first therapy and a second therapy, as compared to treatment with the first therapy without the second therapy. In some embodiments, the first therapy is IO therapy. In some embodiments, the second therapy is a chemotherapy. In some embodiments, the survival is the OS. In some embodiments, the survival is the PFS.

Also provided herein are methods of monitoring, evaluating, or screening an individual having a cancer, comprising acquiring knowledge of a tumor shed value for a liquid biopsy sample obtained from the individual. In some embodiments, the tumor shed value for the liquid biopsy sample obtained from the individual is equal to or greater than a reference tumor shed value. In some embodiments, responsive to the knowledge that the tumor shed value for the liquid biopsy sample obtained from the individual is equal to or greater than a reference tumor shed value, the individual is predicted to have longer survival when treated with a first therapy and a second therapy, as compared to treatment the first therapy without the second therapy. In some embodiments, the first therapy is an IO therapy. In some embodiments, the second therapy is a chemotherapy. In some embodiments, the survival is the OS. In some embodiments, the survival is the PFS.

Also provided herein are methods of stratifying an individual with a cancer for treatment with a first therapy and a second therapy comprising determining a tumor shed value for a liquid biopsy sample obtained from the individual. In some embodiments, if the tumor shed value is equal to or greater than a reference tumor shed value, the method comprises identifying the individual as a candidate for receiving a first therapy and a second therapy. In some embodiments, if the tumor shed value is less than a reference tumor shed value, the method comprises identifying the individual as a candidate for receiving the first therapy without the second therapy. In some embodiments, the first therapy is an (IO) therapy. In some embodiments, the second therapy is a chemotherapy.

Also provided herein are methods of assessing a biomarker in a liquid biopsy sample from an individual having cancer. In some embodiments, the method comprises determining a tumor shed value for the individual. In some embodiments, the method comprises further analyzing the biomarker if the tumor shed value is equal to or greater than a reference tumor shed value. Provided herein are methods comprising tumor fractions

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N. Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

Certain terms are defined. Additional terms are defined throughout the specification.

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

It is understood that aspects and embodiments of the invention described herein include “comprising,” “consisting,” and “consisting essentially of” aspects and embodiments.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2-0-methyl-, 2-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(0)R, P(0)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

The term “detection” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy, e.g., a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations, or other mutations or alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The terms “concurrently” or “in combination” are used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sequence” or “acquiring a read” as the term is used herein, refers to obtaining possession of a nucleotide sequence or amino acid sequence, by “directly acquiring” or “indirectly acquiring” the sequence or read. “Directly acquiring” a sequence or read means performing a process (e.g., performing a synthetic or analytical method) to obtain the sequence, such as performing a sequencing method (e.g., a Next-generation Sequencing (NGS) method). “Indirectly acquiring” a sequence or read refers to receiving information or knowledge of, or receiving, the sequence from another party or source (e.g., a third-party laboratory that directly acquired the sequence). The sequence or read acquired need not be a full sequence, e.g., sequencing of at least one nucleotide, or obtaining information or knowledge, that identifies one or more of the alterations disclosed herein as being present in a sample, biopsy or subject constitutes acquiring a sequence.

Directly acquiring a sequence or read includes performing a process that includes a physical change in a physical substance, e.g., a starting material, such as a sample described herein. Exemplary changes include making a physical entity from two or more starting materials, shearing or fragmenting a substance, such as a genomic DNA fragment; separating or purifying a substance (e.g., isolating a nucleic acid sample from a tissue); combining two or more separate entities into a mixture, performing a chemical reaction that includes breaking or forming a covalent or non-covalent bond. Directly acquiring a value includes performing a process that includes a physical change in a sample or another substance as described above. The size of the fragment (e.g., the average size of the fragments) can be 2500 bp or less, 2000 bp or less, 1500 bp or less, 1000 bp or less, 800 bp or less, 600 bp or less, 400 bp or less, or 200 bp or less. In some embodiments, the size of the fragment (e.g., cfDNA) is between about 150 bp and about 200 bp (e.g., between about 160 bp and about 170 bp). In some embodiments, the size of the fragment (e.g., DNA fragments from liquid biopsy samples) is between about 150 bp and about 250 bp. In some embodiments, the size of the fragment (e.g., cDNA fragments obtained from RNA in liquid biopsy samples) is between about 100 bp and about 150 bp.

“Alteration” or “altered structure” as used herein, of a gene or gene product (e.g., a marker gene or gene product) refers to the presence of a mutation or mutations within the gene or gene product, e.g., a mutation, which affects integrity, sequence, structure, amount or activity of the gene or gene product, as compared to the normal or wild-type gene. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, an alteration which is associated with cancer, or predictive of responsiveness to anti-cancer therapeutics, can have an altered nucleotide sequence (e.g., a mutation), amino acid sequence, chromosomal translocation, intra-chromosomal inversion, copy number, expression level, protein level, protein activity, epigenetic modification (e.g., methylation or acetylation status, or post-translational modification, in a cancer tissue or cancer cell, as compared to a normal, healthy tissue or cell. Exemplary mutations include, but are not limited to, point mutations (e.g., silent, missense, or nonsense), deletions, insertions, inversions, duplications, amplification, translocations, inter- and intra-chromosomal rearrangements. Mutations can be present in the coding or non-coding region of the gene. In certain embodiments, the alteration(s) is detected as a rearrangement, e.g., a genomic rearrangement comprising one or more introns or fragments thereof (e.g., one or more rearrangements in the 5′- and/or 3′-UTR). In certain embodiments, the alterations are associated (or not associated) with a phenotype, e.g., a cancerous phenotype (e.g., one or more of cancer risk, cancer progression, cancer treatment or resistance to cancer treatment). In one embodiment, the alteration (or tumor mutational burden) is associated with one or more of: a genetic risk factor for cancer, a positive treatment response predictor, a negative treatment response predictor, a positive prognostic factor, a negative prognostic factor, or a diagnostic factor.

As used herein, the term “indel” refers to an insertion, a deletion, or both, of one or more nucleotides in a nucleic acid of a cell. In certain embodiments, an indel includes both an insertion and a deletion of one or more nucleotides, where both the insertion and the deletion are nearby on the nucleic acid. In certain embodiments, the indel results in a net change in the total number of nucleotides. In certain embodiments, the indel results in a net change of about 1 to about 50 nucleotides.

“Mutant allele frequency” (MAF) as that term is used herein, refers to the relative frequency of a mutant allele at a particular locus, e.g., in a sample. In some embodiments, a mutant allele frequency is expressed as a fraction or percentage.

“Subgenomic interval” as that term is used herein, refers to a portion of genomic sequence. In an embodiment, a subgenomic interval can be a single nucleotide position, e.g., a variant at the position is associated (positively or negatively) with a tumor phenotype. In an embodiment, a subgenomic interval comprises more than one nucleotide position. Such embodiments include sequences of at least 2, 5, 10, 50, 100, 150, or 250 nucleotide positions in length. Subgenomic intervals can comprise an entire gene, or a portion thereof, e.g., the coding region (or portions thereof), an intron (or portion thereof) or exon (or portion thereof). A subgenomic interval can comprise all or a part of a fragment of a naturally occurring, e.g., genomic DNA, nucleic acid. E.g., a subgenomic interval can correspond to a fragment of genomic DNA which is subjected to a sequencing reaction. In an embodiment, a subgenomic interval is continuous sequence from a genomic source. In an embodiment, a subgenomic interval includes sequences that are not contiguous in the genome, e.g., subgenomic intervals in cDNA can include exon-exon junctions formed as a result of splicing. In an embodiment, the subgenomic interval comprises a tumor nucleic acid molecule. In an embodiment, the subgenomic interval comprises a non-tumor nucleic acid molecule.

In an embodiment, a subgenomic interval comprises or consists of: a single nucleotide position; an intragenic region or an intergenic region; an exon or an intron, or a fragment thereof, typically an exon sequence or a fragment thereof; a coding region or a non-coding region, e.g., a promoter, an enhancer, a 5′ untranslated region (5′ UTR), or a 3′ untranslated region (3′ UTR), or a fragment thereof; a cDNA or a fragment thereof; an SNP; a somatic mutation, a germline mutation or both; an alteration, e.g., a point or a single mutation; a deletion mutation (e.g., an in-frame deletion, an intragenic deletion, a full gene deletion); an insertion mutation (e.g., intragenic insertion); an inversion mutation (e.g., an intra-chromosomal inversion); an inverted duplication mutation; a tandem duplication (e.g., an intrachromosomal tandem duplication); a translocation (e.g., a chromosomal translocation, a non-reciprocal translocation); a rearrangement (e.g., a genomic rearrangement (e.g., a rearrangement of one or more introns, a rearrangement of one or more exons, or a combination and/or a fragment thereof; a rearranged intron can include a 5′- and/or 3′-UTR)); a change in gene copy number; a change in gene expression; a change in RNA levels; or a combination thereof. The “copy number of a gene” refers to the number of DNA sequences in a cell encoding a particular gene product. Generally, for a given gene, a mammal has two copies of each gene. The copy number can be increased, e.g., by gene amplification or duplication, or reduced by deletion.

“Subject interval”, as that term is used herein, refers to a subgenomic interval or an expressed subgenomic interval. In an embodiment, a subgenomic interval and an expressed subgenomic interval correspond, meaning that the expressed subgenomic interval comprises sequence expressed from the corresponding subgenomic interval. In an embodiment, a subgenomic interval and an expressed subgenomic interval are non-corresponding, meaning that the expressed subgenomic interval does not comprise sequence expressed from the non-corresponding subgenomic interval, but rather corresponds to a different subgenomic interval. In an embodiment, a subgenomic interval and an expressed subgenomic interval partially correspond, meaning that the expressed subgenomic interval comprises sequence expressed from the corresponding subgenomic interval and sequence expressed from a different corresponding subgenomic interval.

As used herein, the term “library” refers to a collection of nucleic acid molecules. In one embodiment, the library includes a collection of nucleic acid nucleic acid molecules, e.g., a collection of whole genomic, subgenomic fragments, cDNA, cDNA fragments, RNA, e.g., mRNA, RNA fragments, or a combination thereof. Typically, a nucleic acid molecule is a DNA molecule, e.g., genomic DNA or cDNA. A nucleic acid molecule can be fragmented, e.g., sheared or enzymatically prepared, genomic DNA. Nucleic acid molecules comprise sequence from a subject and can also comprise sequence not derived from the subject, e.g., an adapter sequence, a primer sequence, or other sequences that allow for identification, e.g., “barcode” sequences. In one embodiment, a portion or all of the library nucleic acid molecules comprises an adapter sequence. The adapter sequence can be located at one or both ends. The adapter sequence can be useful, e.g., for a sequencing method (e.g., an NGS method), for amplification, for reverse transcription, or for cloning into a vector. The library can comprise a collection of nucleic acid molecules, e.g., a target nucleic acid molecule (e.g., a tumor nucleic acid molecule, a reference nucleic acid molecule, or a combination thereof). The nucleic acid molecules of the library can be from a single individual. In embodiments, a library can comprise nucleic acid molecules from more than one subject (e.g., 2, 3, 4, 5, 6, 7, 8, 9, 10, 20, 30 or more subjects), e.g., two or more libraries from different subjects can be combined to form a library comprising nucleic acid molecules from more than one subject. In one embodiment, the subject is a human having, or at risk of having, a cancer or tumor.

“Complementary” refers to sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

“Likely to” or “increased likelihood,” as used herein, refers to an increased probability that an item, object, thing or person will occur. Thus, in one example, a subject that is likely to respond to treatment has an increased probability of responding to treatment relative to a reference subject or group of subjects.

“Unlikely to” refers to a decreased probability that an event, item, object, thing or person will occur with respect to a reference. Thus, a subject that is unlikely to respond to treatment has a decreased probability of responding to treatment relative to a reference subject or group of subjects.

“Next-generation sequencing” or “NGS” or “NG sequencing” as used herein, refers to any sequencing method that determines the nucleotide sequence of either individual nucleic acid molecules (e.g., in single molecule sequencing) or clonally expanded proxies for individual nucleic acid molecules in a high throughput fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next-generation sequencing methods are known in the art, and are described, e.g., in Metzker, M. (2010) Nature Biotechnology Reviews 11:31-46, incorporated herein by reference. Next-generation sequencing can detect a variant present in less than 5% or less than 1% of the nucleic acids in a sample.

“Nucleotide value” as referred herein, represents the identity of the nucleotide(s) occupying or assigned to a nucleotide position. Typical nucleotide values include: missing (e.g., deleted); additional (e.g., an insertion of one or more nucleotides, the identity of which may or may not be included); or present (occupied); A; T; C; or G. Other values can be, e.g., not Y, wherein Y is A, T, G, or C; A or X, wherein X is one or two of T, G, or C; T or X, wherein X is one or two of A, G, or C; G or X, wherein X is one or two of T, A, or C; C or X, wherein X is one or two of T, G, or A; a pyrimidine nucleotide; or a purine nucleotide. A nucleotide value can be a frequency for 1 or more, e.g., 2, 3, or 4, bases (or other value described herein, e.g., missing or additional) at a nucleotide position. E.g., a nucleotide value can comprise a frequency for A, and a frequency for G, at a nucleotide position.

“Or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

A “control nucleic acid” or “reference nucleic acid” as used herein, refers to nucleic acid molecules from a control or reference sample. Typically, it is DNA, e.g., genomic DNA, or cDNA derived from RNA, not containing the alteration or variation in the gene or gene product. In certain embodiments, the reference or control nucleic acid sample is a wild-type or a non-mutated sequence. In certain embodiments, the reference nucleic acid sample is purified or isolated (e.g., it is removed from its natural state). In other embodiments, the reference nucleic acid sample is from a blood control, a normal adjacent tissue (NAT), or any other non-cancerous sample from the same or a different subject. In some embodiments, the reference nucleic acid sample comprises normal DNA mixtures. In some embodiments, the normal DNA mixture is a process matched control. In some embodiments, the reference nucleic acid sample has germline variants. In some embodiments, the reference nucleic acid sample does not have somatic alterations, e.g., serves as a negative control.

“Threshold value,” as used herein, is a value that is a function of the number of reads required to be present to assign a nucleotide value to a subject interval (e.g., a subgenomic interval or an expressed subgenomic interval). E.g., it is a function of the number of reads having a specific nucleotide value, e.g., “A,” at a nucleotide position, required to assign that nucleotide value to that nucleotide position in the subgenomic interval. The threshold value can, e.g., be expressed as (or as a function of) a number of reads, e.g., an integer, or as a proportion of reads having the value. By way of example, if the threshold value is X, and X+1 reads having the nucleotide value of “A” are present, then the value of “A” is assigned to the position in the subject interval (e.g., subgenomic interval or expressed subgenomic interval). The threshold value can also be expressed as a function of a mutation or variant expectation, mutation frequency, or of Bayesian prior. In an embodiment, a mutation frequency would require a number or proportion of reads having a nucleotide value, e.g., A or G, at a position, to call that nucleotide value. In embodiments the threshold value can be a function of mutation expectation, e.g., mutation frequency, and tumor type. E.g., a variant at a nucleotide position could have a first threshold value if the patient has a first tumor type and a second threshold value if the patient has a second tumor type.

As used herein, “target nucleic acid molecule” refers to a nucleic acid molecule that one desires to isolate from the nucleic acid library. In one embodiment, the target nucleic acid molecules can be a tumor nucleic acid molecule, a reference nucleic acid molecule, or a control nucleic acid molecule, as described herein.

“Tumor nucleic acid molecule,” or other similar term (e.g., a “tumor or cancer-associated nucleic acid molecule”), as used herein refers to a nucleic acid molecule having sequence from a tumor cell. The terms “tumor nucleic acid molecule” and “tumor nucleic acid” may sometimes be used interchangeably herein. In one embodiment, the tumor nucleic acid molecule includes a subject interval having a sequence (e.g., a nucleotide sequence) that has an alteration (e.g., a mutation) associated with a cancerous phenotype. In other embodiments, the tumor nucleic acid molecule includes a subject interval having a wild-type sequence (e.g., a wild-type nucleotide sequence). For example, a subject interval from a heterozygous or homozygous wild-type allele present in a cancer cell. A tumor nucleic acid molecule can include a reference nucleic acid molecule. Typically, it is DNA, e.g., genomic DNA, or cDNA derived from RNA, from a sample. In certain embodiments, the sample is purified or isolated (e.g., it is removed from its natural state). In some embodiments, the tumor nucleic acid molecule is a cfDNA. In some embodiments, the tumor nucleic acid molecule is a ctDNA. In some embodiments, the tumor nucleic acid molecule is DNA from a CTC.

“Variant,” as used herein, refers to a structure that can be present at a subgenomic interval that can have more than one structure, e.g., an allele at a polymorphic locus.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as an RNA molecule or a cDNA molecule, can be substantially free of other cellular material or culture medium, e.g., when produced by recombinant techniques, or substantially free of chemical precursors or other chemicals, e.g., when chemically synthesized.

### III. Methods of Determining Tumor Fractions

Provided herein are methods comprising determining a tumor shed value in a liquid biopsy sample from an individual. In some embodiments, determining a tumor shed value comprises determining a composite tumor fraction (cTF). In some embodiments, determining a tumor shed value comprises a tumor fraction estimator (TFE) process. In some embodiments, determining a tumor shed value comprises determining a maximum allele fraction (MAF).

### A. Tumor Fraction Determination

In some embodiments, the methods of the disclosure comprise determining a tumor shed value in a liquid biopsy sample of an individual.

As used herein, “tumor shed” or “tumor fraction” are a measure of tumor genomic content, for example in a liquid biopsy sample, in proportion to the total genomic content regardless of cell origin. In general, it is advantageous to determine (e.g., estimate) tumor content, or a change in tumor content, from a sample, since this can aid in both reporting alterations and informing on disease presence or progression. For example, liquid biopsies, which typically utilize blood samples from cancer patients, can be useful when solid biopsies are not possible or recommended. In some embodiments, tumor fraction in a cell-free sample comprises a measure of the tumor DNA that has shed into the vasculature or lymphatics from a primary tumor relative to the amount of total DNA (e.g., tumor and normal) shed into the blood stream, and is being carried around the body in the blood circulation. Tumor fraction can be used to monitor a patient at risk for cancer (with or without current diagnosis); as a factor used in diagnosing cancer; or to determine if a current treatment regimen is having an effect, e.g., a beneficial effect.

Traditional processes for measuring tumor fraction typically require that both purity and ploidy, modeled parameters, be inferred from either log ratio and allele frequency measurements or both, or from pathology review. In some embodiments, tumor fraction can be considered as a modeled parameter of the fraction of cancer cells in a heterogeneous tumor sample and can take into account tumor purity or other measures. In some embodiments, tumor cell ploidy can refer to the average weighted copy number of all chromosomes (or portions thereof). The ploidy observed in a sample can be impacted by the varying degrees of aneuploidy of tumor cells, the heterogeneity of the sample (e.g., different ratios of tumor cells to normal cells), or both.

Traditional processes for predicting tumor fraction can be highly unreliable for low tumor content due to poorly fit models. In some embodiments, tumor fraction (and associated confidence levels) are determined based on the effects of tumor cell aneuploidy, e.g., as measured by the allele coverage or allele fraction at one or more subgenomic intervals in a sample. In some embodiments, the subgenomic interval comprises a heterozygous single nucleotide polymorphism (SNP) site. In other embodiments, the subgenomic interval comprises more than one nucleotide positions. The term “allele coverage,” or simply “coverage” or “Cvg” as used herein, refers to the number of reads (e.g., unique reads) generated from DNA sequencing of a subgenomic interval in a sample. The term “allele intensity,” or simply “intensity,” as used herein, refers to the number of signals (e.g., unique signals) generated from a genomic hybridization at a subgenomic interval in a sample. It will be appreciated that “reads” or “signal” is intended to encompass situations in which there may exist duplicates of the same “unique read” or “unique signal” (i.e., duplicates are not removed prior to performing the methods described herein), but any ratios calculated using the described methods will yield a value very similar to “unique” read or signal ratios, since the duplicates will be represented in both the numerator and denominator.

The term “allele fraction,” as used herein, refers to the relative level (e.g., abundance) of an allele at a subgenomic interval in a sample. Allele fraction can be expressed as a fraction or percentage. For example, allele fraction can be expressed as the ratio of the number of one particular allele (e.g., A, T, C, or G) at a subgenomic interval relative to the number of all different alleles at that subgenomic interval. In some embodiments, allele fraction is measured by determining the ratio of the coverage or intensity from one particular allele (e.g., A, T, C, or G) to the total coverage or intensity from all different alleles at a given subgenomic interval. Sometimes, the terms “allele fraction” and “allele frequency” are used interchangeably herein. As used herein, a log ratio is typically measured by log 2 (T/R), where T is the level (e.g., abundance) of one or more alleles associated with a subgenomic interval in a sample, and R is the level (e.g., abundance) of the one or more alleles associated with the subgenomic interval in a reference sample. The term, “allele,” as used herein, refers to one of the two or more alternative forms of a genomic sequence (e.g., a gene or any portion thereof). For example, if a “C” to “T” SNP is associated with a subgenomic interval, then the subgenomic interval can be described as being associated with alleles “C” and “T” with respect to the SNP.

In some embodiments, there are two or more different alleles associated with a subgenomic interval. If the two or more different alleles are present in a sample, the subgenomic interval is considered as heterozygous for the sample. If the subgenomic interval is not heterozygous for the sample, it can, in some embodiments, be homozygous, semizygous, or hemizygous.

The term, “abundance,” as used herein, refers to the amount, number, or quantity of an object. For example, the abundance of an allele associated with a subgenomic interval can mean the amount, number, or quantity of an allele associated with a subgenomic interval in a sample, for example, as determined by sequencing or array-based comprehensive genomic hybridization (aCGH). For example, if there are two alleles, “A” and “G,” associated with a particular subgenomic interval, and there are 10 copies of allele “A” and 20 copies of allele “G” in a sample, the abundance of allele “A” can be considered as 10 and the abundance of allele “G” can be considered as 20. In some embodiments, the abundance of an allele is measured by allele coverage or allele intensity. For example, the number of unique reads for allele “A” or “G” reflects how many copies of allele “A” or “G” are present in the sample.

The term “certainty metric,” as used herein, refers to a metric derived from a measure or value of a target variable. In some embodiments, the target variable may represent an abundance of a subgenomic interval, or an allele associated with the subgenomic interval, in a sample. In some examples, the certainty metric may be a deviation of an allele fraction from an expected allele fraction. In other examples, the certainty metric may be a measure of allele intensity. These examples are intended to be illustrative, and other certainty metrics may be used.

As an example, for a heterozygous SNP, an allele fraction value of 0.50 can indicate a typical diploid subgenomic interval; and an allele fraction that deviates from an expected value of 0.50 indicates aneuploidy at that site. In these examples, this deviation of allele coverage can be correlated with tumor fraction in a training set in order to build a model that determines (e.g., predicts or estimates) tumor fraction based on allele coverage. In some embodiments, the methods described herein correlate deviation of allele fraction or log ratio with tumor fraction, thereby eliminating the need to model tumor purity and ploidy. In some embodiments, the methods described herein allow for more accurate determination of tumor fraction of low level, e.g., less than 30%. In an embodiment, the allele fraction or log ratio is determined by a method comprising sequencing, e.g., next generation sequencing (NGS). It will be appreciated that the methods for determining allele fraction or log ratio are not limited to sequencing. Any method that measures, for example, SNP coverage or relative level (e.g., abundance) of SNPs, as well as, any method that measures coverage from larger genomic regions can be used. In an embodiment, the allele fraction or log ratio is determined by a method other than sequencing, e.g., is determined by an array-based comprehensive genomic hybridization (aCGH). In an embodiment, the tumor fraction is, or is expected to be, less than or equal to 0.25, less than or equal to 0.2, less than or equal to 0.15, or less than or equal to 0.1, e.g., between 0.1 and 0.3, between 0.1 and 0.2, between 0.2 and 0.3, or between 0.15 and 0.25.

While in some embodiments the methods described herein use allele fraction or log ratio to indicate expected coverage proportions, it will be appreciated that the present disclosure is generally intended to describe the correlation of tumor fraction to expected coverage deviations, without limitation to allele fraction, log ratio, or any other specific metric.

As used herein, a “single-nucleotide polymorphism,” or SNP, refers to an alteration of a single nucleotide that occurs at a specific position in the genome. In some embodiments, such alteration is present to some appreciable degree within a population (e.g., >1%). Typically, a SNP is a germline alteration and is not a somatic single-nucleotide variant (SNV).

In an embodiment, the tumor fraction is a numerical representation (e.g., fraction or percentage) indicating the amount of DNA from tumor cells versus the total amount of DNA (e.g., tumor and non-tumor DNA) in a liquid biopsy sample. In an embodiment, the tumor fraction in a liquid biopsy sample indicates the presence or level of detectable tumor in the body.

An exemplary method of determining a tumor fraction of a sample from a subject includes: acquiring a plurality of values, each value indicative of an allele fraction at a corresponding locus within a subgenomic interval in the sample; determining a certainty metric indicative of a dispersion of the plurality of values; accessing a predetermined relationship between a stored certainty metric and a stored tumor fraction; and determining, from the certainty metric and the predetermined relationship, the tumor fraction of the sample

A value indicative of an allele fraction can be determined for each corresponding locus. The loci include may include one or more nucleotide. In some embodiments, the corresponding loci comprise one or more loci having a different maternal allele and paternal allele. In some embodiments, the corresponding loci consist of loci having a different maternal allele and paternal allele. In some embodiments, the corresponding loci comprise one or more loci having the same maternal allele and paternal allele.

In some embodiments, the plurality of values indicative of an allele fraction at a plurality of corresponding loci in the sample is a plurality of allele fractions at the plurality of corresponding loci in the sample. The allele fraction at each of the corresponding loci may be determined, for example, by sequencing nucleic acid molecules in the tumor sample and assigning an allele coverage for each allele at each locus. For example, the allele fraction at locus i (afᵢ) may be determined by:

$${af}_{i} = \frac{{Cvg}_{i , a}}{{Cvg}_{i , a} + {Cvg}_{i , b}}$$

wherein Cvg_{i,a} is the coverage of allele a at locus i, and Cvg_{i,b} is the coverage of allele b at locus i. In some embodiments, allele a and allele b are assigned such that Cvg_{i,a}≤Cvg_{i,b}, such that afᵢ≤0.5.

In some embodiments, the expected allele fraction is the allele fraction expected in a healthy individual or healthy sample (i.e., a non-tumor sample). For example, the allele fraction at a heterozygous locus (that is, having a different maternal allele and paternal allele) is expected to be 0.5, and the allele fraction at a homozygous locus (that is, wherein the maternal allele and the paternal allele are the same) is expected to be 1.0.

Allele fraction is an exemplary value for determining tumor fraction according to the methods described herein, although other values indicative of allele fraction may be used in some embodiments. In some embodiments, the value indicative of the allele fraction is a relative difference in allele frequency. For example, the value indicative of the allele fraction may be ratio of the difference in the abundance (e.g., a coverage or sequencing depth) between a maternal allele and a paternal allele relative to the abundance of the maternal allele or the paternal allele. That is, in some embodiments, the value can a relative_difference as de

$$relative\_difference = \frac{{Cvg}_{i , a} - {Cvg}_{i , b}}{{Cvg}_{i , b}}$$

wherein Cvg_{i,a} is the coverage of allele a at locus i, and Cvg_{i,b} is the coverage of allele b at locus i. In a healthy individual or healthy sample, the difference between the allele frequency, as well as the relative difference, is expected to be 0. In some embodiments, a probability distribution function is determined for the plurality of values indicative of allele fraction. For example, in some embodiments, the probability distribution function is determined for the plurality of allele fractions at the plurality of corresponding loci in the sample. In some embodiments, the probability distribution function for the plurality of allele fractions is defined by:

$$P ( af ) = P ( \frac{{Cvg}_{i , a}}{{Cvg}_{i , a} + {Cvg}_{i , b}} )$$

wherein Cvg_{i,a} is the coverage of allele a at locus i, and Cvg_{i,b} is the coverage of allele b at locus i.

The dispersion (or certainty metric) can be, for example, a deviation from the expected allele fraction (or value indicative of expected allele fraction) across the plurality of loci. In some embodiments, the certainty metric is a root mean squared deviation from the expected allele fraction (or value indicative thereof). For example, in some embodiments, the certainty metric is a root mean squared deviation (RMSD) defined by:

$$RMSD = {[ \frac{1}{N} {\sum }_{i = 0}^{N} {( {af}_{i} - {af}_{i , expected} )}^{2} ]}^{( 1 / 2 )}$$

wherein afᵢ is the allele frequency (or value indicative of the allele frequency, such as a relative difference ratio) at locus i, af_{expected} is the expected allele frequency at locus i, and N is the number of loci in the plurality of corresponding loci. For example, for some loci, af_{expected} may be 0.5, and at other loci af_{expected} may be 1. In some embodiments, the loci include only those loci having a different maternal allele and paternal allele. Thus, the af_{expected} may be defined as 0.5 across all loci, and the RMSD can be defined as:

$$RMSD = {[ \frac{1}{N} {\sum }_{i = 0}^{N} {( {af}_{i} - 0.5 )}^{2} ]}^{( 1 / 2 )}$$

In some embodiments, the value indicative of the allele fraction may be ratio of the difference in abundance (e.g., a coverage or sequencing depth) between a maternal allele and a paternal allele, relative to the abundance of the maternal allele or the paternal allele, and the af_{expected} may be defined as 0. Thus, the RMSD can be defined as:

$$RMSD = {[ \frac{1}{N} {\sum }_{i = 0}^{N} {( \frac{{Cvg}_{i , a} - {Cvg}_{i , b}}{{Cvg}_{i , b}} )}^{2} ]}^{( 1 / 2 )}$$

wherein Cvg_{i,a} is the coverage of allele a at locus i, and Cvg_{i,b} is the coverage of allele b at locus i.

In some embodiments, a probability distribution (e.g., a probability distribution function) can be determined for allele fractions across a plurality of loci. The certainty metric (e.g., a dispersion) can be a metric of the probability distribution, such as an entropy of the probability distribution. For example, in some embodiments, the entropy of an allele fraction probability distribution function (S[P(af)]) may be defined as:

$$S [ P ( af ) ] = {\sum }_{af = 0}^{0.5} P ( af ) {log}_{n} ( P ( af ) )$$

wherein P(af) is the allele fraction probability distribution function, and n is the log base. In some embodiments, the log base is 2 (i.e., log 2). Accordingly, in some embodiments, the entropy of an allele fraction probability distribution function (S[P(af)]) may be defined as:

$$S [ P ( af ) ] = {\sum }_{af = 0}^{0.5} P ( af ) {log}_{2} ( P ( af ) )$$

In some embodiments, a method of determining a tumor fraction of a sample from a subject, the method comprising: acquiring a plurality of values, each value indicative of a difference between an allele coverage of a locus in a tumor sample and an allele coverage of the same locus in a non-tumor sample at a plurality of loci within a subgenomic interval; determining a certainty metric indicative of a dispersion of the plurality of values; accessing a predetermined relationship between a stored certainty metric and a stored tumor fraction; and determining, from the certainty metric and the predetermined relationship, the tumor fraction of the sample. In some embodiments, the tumor sample and the non-tumor sample are obtained from the same individual (i.e., a matched normal control). In some embodiments, the tumor sample and the non-tumor sample are obtained from different individuals. The coverage may be a raw coverage (for example, a raw number of sequencing reads), a normalized coverage (for example, normalized to a mean or median sequencing depth), and/or otherwise bias-corrected coverage (for example, a GC-bias corrected coverage depth). In some embodiments, the allele coverage comprises the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele).

In some embodiments, each value indicative of the difference between an allele coverage of the locus in a tumor sample and an allele coverage of the same locus in the non-tumor sample comprises a ratio of the allele coverage of a locus in the tumor sample compared to the allele coverage of the same locus in the non-tumor sample. In some embodiments, the allele coverage comprises the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele). For example, in some embodiments, the ratio may be defined as:

$$ratio = \frac{( {Cvg}_{i , a}^{cancer} + {Cvg}_{i , b}^{cancer} )}{( {Cvg}_{i , a}^{normal} + {Cvg}_{i , b}^{normal} )}$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample.

In some embodiments, each value indicative of the difference between an allele coverage of the locus in a tumor sample and an allele coverage of the same locus in the non-tumor sample is a log ratio (such as a log 2 ratio) of the allele coverage of a locus in the tumor sample compared to the allele coverage of the same locus in the non-tumor sample. In some embodiments, the allele coverage comprises the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele). For example, the log ratio may be defined, in some embodiments, as:

$${log}_{n} ratio = {log}_{n} \frac{( {Cvg}_{i , a}^{cancer} + {Cvg}_{i , b}^{cancer} )}{( {Cvg}_{i , a}^{normal} + {Cvg}_{i , b}^{normal} )}$$

wherein logₙ is the log at base n, Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample. For example, the log ratio may be a log₂ ratio. In some embodiments, the log ratio is defined as:

$${log}_{2} ratio = {log}_{2} \frac{( {Cvg}_{i , a}^{cancer} + {Cvg}_{i , b}^{cancer} )}{( {Cvg}_{i , a}^{normal} + {Cvg}_{i , b}^{normal} )}$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample

In some embodiments, each value indicative of the difference between an allele coverage of the locus in a tumor sample and an allele coverage of the same locus in the non-tumor sample comprises a ratio of the difference between the allele coverage of a locus in the tumor sample compared to the allele coverage of the same locus in the non-tumor sample, relative to the allele coverage of the same locus in the non-tumor sample. In some embodiments, the allele coverage comprise the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele). For example, in some embodiments, the ratio is defined as:

$$\frac{( {Cvg}_{i , a}^{cancer} + {Cvg}_{i , b}^{cancer} ) - ( {Cvg}_{i , a}^{normal} + {Cvg}_{i , b}^{normal} )}{( {CCvg}_{i , a}^{normal} + {Cvg}_{i , b}^{normal} )}$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample.

In some embodiments, a probability distribution function is determined for the plurality of values indicative of the difference between an allele coverage of the locus in a tumor sample and an allele coverage of the same locus in the non-tumor sample. In some embodiments, the allele coverage comprises the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele). For example, in some embodiments, the probability distribution function is determined for the plurality of ratios of the allele coverage of a locus in the tumor sample compared to the allele coverage of the same locus in the non-tumor sample (such as a log ratio, for example a log₂ ratio). In some embodiments, the probability distribution function for the plurality of allele fractions is defined by:

$$P ( {log}_{n} ( \frac{{Cvg}_{i , a}^{cancer} + {Cvg}_{i , b}^{cancer}}{{Cvg}_{i , a}^{normal} + {Cvg}_{i , b}^{normal}} ) )$$

wherein logₙ is the log at base n, Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample. In some embodiment, log ratio is a log₂ ratio. For example, in some embodiments, the probability distribution function for the plurality of allele fractions is defined by:

$$P ( {log}_{2} ( \frac{{Cvg}_{i , a}^{cancer} + {Cvg}_{i , b}^{cancer}}{{Cvg}_{i , a}^{normal} + {Cvg}_{i , b}^{normal}} ) )$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample.

The dispersion (or certainty metric) can be, for example, a deviation of each value within the plurality of values from an expected value across the corresponding loci. The expected value is the value that would be expected if the tumor sample were non-tumor (e.g., a healthy) sample. In some embodiments, the certainty metric is a root mean squared deviation from the expected value. For example, in some embodiments, the certainty metric is a root mean squared deviation (RMSD) defined by:

$$RMSD = \sqrt{\frac{1}{N} {\sum }_{i}^{N} {[ ( {log}_{2} \frac{( {Cvg}_{i , a}^{cancer} + {Cvg}_{i , b}^{cancer} )}{( {Cvg}_{i , a}^{normal} + {Cvg}_{i , b}^{normal} )} ) - 0 ]}^{2}}$$

In some embodiments, the value indicative of the allele fraction is a ratio of the difference between the allele coverage of a locus in the tumor sample compared to the allele coverage of the same locus in the non-tumor sample, relative to the allele coverage of the same locus in the non-tumor sample. Thus, the RMSD can be defined as:

$$RMSD = {[ \frac{1}{N} {\sum }_{i = 0}^{N} {( \frac{( {Cvg}_{i , a}^{cancer} + {Cvg}_{i , b}^{cancer} ) - ( {Cvg}_{i , a}^{normal} + {Cvg}_{i , b}^{normal} )}{( {CCvg}_{i , a}^{normal} + {Cvg}_{i , b}^{normal} )} )}^{2} ]}^{( 1 / 2 )}$$

In some embodiments, a probability distribution (e.g., a probability distribution function) can be determined for the plurality of values indicative of the difference between an allele coverage of the locus in a tumor sample and an allele coverage of the same locus in the non-tumor sample. The certainty metric (e.g., a dispersion) can be a metric of the probability distribution, such as an entropy of the probability distribution. For example, in some embodiments, the entropy of an allele fraction probability distribution function (S[P(af)]) may be defined as:

$$S [ P ( ln r ) ] = {\sum }_{lnr = 0} P ( ln r ) {log}_{n} ( P ( ln r ) )$$

$$wherein :$$

$$P ( ln r ) = P ( {log}_{n} ( \frac{{Cvg}_{i , a}^{cancer} + {Cvg}_{i , b}^{cancer}}{{Cvg}_{i , a}^{normal} + {Cvg}_{i , b}^{normal}} ) )$$

wherein logₙ is a log having base n, Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample. In some embodiments, the log base is 2 (i.e., log₂). Accordingly, in some embodiments, the entropy of an allele fraction probability distribution function (S[P(af)]) may be defined as:

$$S [ P ( l 2 r ) ] = {\sum }_{lnr = 0} P ( l 2 r ) {log}_{2} ( P ( l 2 r ) )$$

$$P ( l 2 r ) = P ( {log}_{2} ( \frac{{Cvg}_{i , a}^{cancer} + {Cvg}_{i , b}^{cancer}}{{Cvg}_{i , a}^{normal} + {Cvg}_{i , b}^{normal}} ) )$$

wherein:
wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample.

A relationship between one or more stored certainty metrics and one or more stored tumor fractions can be used to determine the tumor fraction based on the determined certainty metrics. In some embodiments, a model is trained to using a training dataset that includes training certainty metrics and associated tumor fractions to determine the relationship between the certainty metrics and the tumor fractions. The training dataset may be determined, for example, using a plurality of clinical samples with known (i.e., training) tumor fractions (for example, as determined by maximum somatic allele frequency (MSAF), which filters germline variant calls from all calls in a tumor sample and compares residual variants (i.e., the maximum somatic variants) to the total variants (maximum somatic variants plus germline variants) to determine the maximum somatic allele frequency). Nucleic acid molecules in the clinical samples can be sequenced to determine allele frequency across a plurality of loci (or a value indicative of an allele frequency), as well as an associated training certainty metric. The training certainty metrics can be correlated with the training tumor fractions to determine the relationship between certainty metric and tumor fraction. In another method, serial dilutions may be made from one or more clinical samples to obtain a plurality of different tumor fractions, which can be correlated with the certainty metric for the serially diluted samples to determine the relationship.

In some embodiments, to determine (e.g., estimate) the tumor fraction, a training subprocess is first performed. A dataset can be constructed from clinical specimens. Using the training set and in-silico dilutions of the training set, tumor fraction can be correlated to variation in allele fractions or log ratios corresponding to aneuploidy typically observed in tumors. In other examples, cell-line/clinical sample dilutions can be performed.

In some embodiments, the certainty metric may be functions of the coverage at particular SNP bins for particular alleles and/or an allele frequency (e.g., in the range of 0 to 0.5). In some examples, the training data uses as input a deviation metric (e.g., allele fraction deviation or log ratio deviation) and returns the estimated tumor fraction, along with lower and upper bounds. Values that deviate from (i.e., fall between) 0 and 1 and not 0.5 (exclusive) may be thought of as “noise,” and the averaged noise may be correlated with an expected or estimated tumor fraction. In other examples, the training data provides as input a log ratio deviation metric, or in general, any metric which quantifies coverage deviations from expectations. In either case, the allele coverage deviation metric or the log ratio deviation metric may be a measure of the tumor fraction.

Utilizing these correlations derived during training, a tumor fraction of a patient can be estimated or evaluated with upper and lower bounds. Coverage metrics, such as SNP allele coverage variation metrics, may be used in generating the correlation.

### (i) Tumor Fraction Estimator

In some embodiments, determining a tumor shed value comprises a tumor fraction estimator (TFE) process. In some embodiments, the TFE process comprises receiving a plurality of values, each value indicative of an allele fraction at a corresponding locus within a subgenomic interval in the sample; determining a certainty metric value indicative of a dispersion of the plurality of values; and determining an estimate of the tumor fraction of the sample based on the certainty metric value for the sample and a predetermined relationship between one or more stored certainty metric values and one or more stored tumor fraction values, wherein the estimate is determined as the tumor fraction of the sample.

In the TFE process, a value for a target variable associated with a subgenomic interval is obtained, e.g., directly obtained, from a sample from a subject. The target variable may be, for example, an allele fraction. The certainty metric may be determined from the target variable, and a determined relationship is accessed between a stored certainty metric and a stored tumor fraction. The determined relationship may include historical sample data (collected from patients or other test subjects) relating a certainty metric (e.g., a sampled allele fraction deviation) for at least one heterozygous SNP site to a corresponding sampled tumor fraction. In some instances, the sampled allele coverage deviation is a “noise” metric, reflecting the degree to which an allele fraction varies from an expected value. In some examples, the number of data points correlating tumor fraction to noise metrics calculated from the allele fraction may exceed one hundred (100), one thousand (1,000), ten thousand (10,000), or more.

In one example, the determined relationship may be derived from an in silico process, and the analysis may be performed by a machine learning process. The process may perform a sample dilution (e.g., using a matched normal) starting at a particular tumor fraction in order to correlate one or more coverage deviation metrics (e.g., allele fraction values) across one or more subgenomic intervals (e.g., SNPs, SNP bins, and/or chromosomes). The metric may be a measure of the frequency and degree to which tumor fraction falls in between the values of 0 or 1. Averaged “noise” metrics between 0 and 1 (exclusive) may be correlated with an expected or estimated tumor fraction. In some embodiments, the disclosed methods may comprise: obtaining a training dataset comprising a plurality of relationships between a plurality of training certainty metric values and associated training tumor fraction values; training a machine learning model based on the training dataset; and using the trained machine learning model to determine a tumor fraction value from the certainty metric value for the sample.

The number of elements associated with subgenomic intervals that contribute to the determination of the certainty metric value, which is correlated to tumor fraction, may be on the order of ten (10), one hundred (100), one thousand (1,000), ten thousand (10,000), or more.

Due to the large number of elements associated with subgenomic intervals that contribute to the certainty metric determination in the correlation, the elements may be “binned” or aggregated by subgenomic interval position or other characteristics in some examples. Binning may avoid a single (or small set of) element(s) disproportionately weighting a correlation in the certainty metric, adversely affecting the estimated tumor fraction. For example, if one element at a single subgenomic interval represents a copy variant with 5,000 copies, it may result in an estimated tumor fraction that is inaccurately high. Therefore, in some examples, elements that contribute to a certainty metric are averaged or otherwise aggregated by chromosome, for example, for each of 22 relevant chromosomes. Those 22 aggregate chromosome values can then be used to calculate the certainty metric which is then correlated with tumor fraction, ensuring that a single subgenomic interval (e.g., SNP site) does not disproportionately affect the correlation. Other methods can be utilized to limit the effect of extreme copy-number events, such as, but not limited to, excluding outlier values from the certainty metric determinations.

In some instances, the correlation may be a mean (i.e., average) correlation, with upper bound correlations and lower bound correlations also calculated. In this way, the mean correlation is bounded by a 95% confidence interval.

The subgenomic interval may comprise one or several subgenomic intervals, and in some examples may be at least one heterozygous SNP site. Subgenomic intervals may be selected based on various criteria. For example, subgenomic intervals may be selected based on how polymorphic the subgenomic interval is in a general healthy population, as well as, healthy subpopulations (including different genders, ages or ethnic backgrounds). It may be advantageous that the subgenomic intervals vary considerably in the healthy population. The sequencing characteristics of the subgenomic intervals may also be selected on the basis of being “well-behaved,” i.e., near expected allele-frequencies, such as 0, 0.5, and 1.0. Furthermore, the regions may be selected on the basis of being “well covered,” i.e., having typical coverage across populations for the site. Subgenomic intervals may be excluded if they occur in simple repeats of gene families or in any generally repeating sequence of DNA, since this characteristic can challenge alignment methodologies. In an embodiment, subgenomic intervals may be located in a genomic region that is free, or essentially free, of high homology, simple repeats, or gene families.

In an embodiment, the subgenomic interval comprises a minor allele. As used herein, a “minor allele” is an allele other than the most common allele (e.g., the second most common allele or the least common allele) associated with a particular subgenomic interval in a given population. In an embodiment, at least 10, 20, 50, 100, 150, 200, 300, 400, 500, 600, 700, 800, 900, 1000, 1200, 1400, 1600, 1800, 2000, or 10000 heterozygous subgenomic intervals are selected. In one example, no more than 10, 20, 50, 100, 150, 200, 300, 400, 500, 600, 700, 800, 900, 1000, 1200, 1400, 1600, 1800, 2000, or 10000 heterozygous SNP sites are selected.

In one example, the selected subgenomic intervals and/or correlation may be universal, i.e., across all disease ontologies, in order to provide a broad screening technique. In other examples, subgenomic intervals may be selected, and the correlation tuned, based on disease ontology (e.g., tumor type).

One or more certainty metrics may be used in correlating a target variable (e.g., allele coverage deviation and/or allele fraction variation) to tumor fraction. For example, metrics relating to allele fraction may be applied. In one example, an allele frequency entropy metric or root mean squared deviation (RMSD) metric may be used:

$$Allele Frequency Entropy : S [ P ( af ) ] = {\sum }_{af = 0}^{0.5} P ( af ) {log}_{2} ( P ( af ) )$$

$$Root Mean Squared Deviation : RMSD = {[ \frac{1}{N} {\sum }_{i = 0}^{N} {( {af}^{i} - 0.5 )}^{2} ]}^{( 1 / 2 )}$$

where i=SNP bin and af-allele frequency on the range of 0 to 0.5. Folded SNP allele frequencies are used here by convention (e.g., as described in Nielsen. Hum Genomics. 2004; 1(3): 218-224 and Marth, et al. Genetics. 2004; 6(1): 351-372), but the methodology holds if the full range of 0 to 1 is utilized. Other metrics may also be used, such as metrics based off the log 2 ratio. Any of these metrics may incorporate factors such as coverage at a particular SNP bin, where the “bin” can be defined to be 1 or more base-pairs. In some embodiments, the certainty metric may be written as a function of coverage, such that certainty_metric=f(Cvg). Further, any mathematical transformation or operation acting on the certainty_metric, may also be considered a certainty_metric.

In some embodiments, the certainty metric may be a deviation from the expected log 2 ratio for at least one subgenomic interval. In other examples, the certainty metric may be a deviation from expected allele fraction in a healthy population for at least one subgenomic interval (e.g., a SNP) that is known to be heterozygous. In other examples, the certainty metric may be a deviation from expected allele coverage in the healthy population for at least one subgenomic interval (e.g., a SNP) that is known to be heterozygous.

Table A shows exemplary certainty metrics that may be used, including any p-moment or combination thereof:

TABLE A

| Conditions to Relate | | |
|---|---|---|
| Metric to Tumor Fraction | Metric Calculating Relative Certainty of Variable | Comments |
| INTRA-SAMPLE- | | |
| Comparisons | | |
| All coverage under | variable = af | Intra-sample comparison |
| consideration is from the cancer sample | $af = \frac{{Cvg}_{a}}{{Cvg}_{a} + {Cvg}_{b}}$ | Cvgₐ < Cvg_{b}, so that af ≤ 0.5 |
| af is a metric which | af_{expectation} = 0.5 | |
| compares coverages from | | |
| the maternal and paternal | | |
| chromosomes | | |
| Cvgₐ is maternal or | | |
| paternal, Cvg_{b} is the other | | |
| allele. | | |
| N is a number of | certainty_metric = | |
| subgenomic intervals, and i is an index of N. | $\sqrt{\frac{1}{N} {\sum }_{i}^{N} {( af - {af}_{expectation} )}^{2}} =$ | |
| Any loci may be used such that the germline maternal and paternal chromosomes differ at the | $\sqrt{\frac{1}{N} {\sum }_{i}^{N} {( \frac{{Cvg}_{i , a}}{{Cvg}_{i , a} + {Cvg}_{i , b}} - 0.5 )}^{2}}$ | |
| same genomic location; | | |
| could be a SNP or | | |
| something larger. | | |
| All coverage under | variable = Cvg | Intra-sample comparison |
| consideration is from the | relative_difference = | Cvgₐ > Cvg_{b} |
| cancer sample Cvgₐ is matemal or | $\frac{{Cvg}_{a} - {Cvg}_{b}}{{Cvg}_{b}}$ | Previous choice is chosen because we assume the allele is amplified and |
| paternal, Cvg_{b} is the other | relative_difference_{expectation} = 0 | thus the ″b″ allele would be the |
| allele | | normal. In general, it should not |
| Any loci may be used such that the germline maternal and paternal chromosomes differ at the same genomic location; | certainty_metric = $\sqrt{\frac{1}{N} {\sum }_{i}^{N} {( \frac{{Cvg}_{ia} - {Cvg}_{ib}}{{Cvg}_{ib}} )}^{2}}$ | matter since this will switch for deletions. Thus, Cvgₐ could be < Cvg_{b}, or one could completely ignore the convention |
| could be a SNP or | | |
| something larger. | | |
| All coverage under | variable = af | Intra-sample comparison |
| consideration is from the | probability of allele frequency = P(af) = | S = Entropy is a metric that |
| cancer sample af is a metric which compares coverages from | $P ( \frac{{Cvg}_{i , a}}{{Cvg}_{i , a} + {Cvg}_{i , b}} )$ | inherently measures relative certainty of the variable |
| the maternal and paternal | | |
| chromosomes | | |
| Cvgₐ is maternal or | certainty_metric = S[P(af)] | |
| paternal, Cvg_{b} is the other | | |
| allele. | | |
| Any loci may be used | | |
| such that the germline | | |
| maternal and paternal | | |
| chromosomes differ at the | | |
| same genomic location; | | |
| could be a SNP of | | |
| something larger. | | |
| INTER-SAMPLE- | | |
| Comparisons | | |
| All coverage under | variable = log2ratio = l2r | Inter-sample comparison |
| consideration is from both the cancer and reference sample ratio is determined from the total coverage from | $ratio = \frac{( {Cvg}_{a}^{cancer} + {Cvg}_{b}^{cancer} )}{( {Cvg}_{a}^{normal} + {Cvg}_{b}^{normal} )}$ l2r = log₂(ratio) l2r_{expectation} = 0 | |
| maternal and paternal from | | |
| cancer sample to the | | |
| maternal and paternal from | | |
| the reference. | | |
| Any loci of the genome | certainty_metric = | |
| from 1 to an infinite set of bases can be used. | $\sqrt{\frac{1}{N} {\sum }_{i}^{N} {[ l 2 r - l 2 {r}_{expectation} ]}^{2}} =$ | |
| | $\sqrt{{\frac{1}{N} {\sum }_{i}^{N} [ {log}_{2} \frac{( {Cvg}_{i , a}^{cancer} + {Cvg}_{i , b}^{cancer} )}{{Cvg}_{i , a}^{normal} + {Cvg}_{i , b}^{normal} )} ) - 0 ]}^{2}}$ | |
| All coverage under | variable = Cvg | Inter-sample comparison |
| consideration is from both | Cvg_{cancer} = (Cvgₐ^{cancer} + Cvg_{b}^{cancer}) | |
| the cancer and reference | Cvgₙₒᵣₘₐₗ = (Cvgₐ^{normal} + Cvg_{b}^{normal}) | |
| sample | | |
| ratio is determined from | relative_difference = | |
| the toal coverage from maternal and paternal from cancer sample to the matemal and paternal from the reference. | $\frac{{Cvg}_{cancer} - {Cvg}_{normal}}{{Cvg}_{normal}}$ relative_difference_{expectaton} = 0 | |
| Any loci of the genome | certainty_metric = | |
| from 1 to an infinite set of bases can be used. | $\sqrt{\frac{1}{N} {\sum }_{i}^{N} ( \frac{{{Cvg}_{i , cancer} - {Cvg}_{i , normal} )}^{2}}{{Cvg}_{i , normal}}}$ | |
| Total coverage from | variable = log2ratio = l2r | Inter-sample comparison |
| maternal and paternal from | probability of log2ratio = P(l2r) = | S = Entropy is a metric that does |
| cancer sample is compared to the maternal and paternal from the reference. Any loci of the genome from 1 to an infinite set of bases can be used | $P ( {log}_{2} ( \frac{{Cvg}_{i , a}^{cancer} + {Cvg}_{i , b}^{cancer} )}{{Cvg}_{i , a}^{normal} + {Cvg}_{i , b}^{normal} )} ) )$ | not calculate a deviation from expectation. It is a metric that inherently measures relative certainty of the variable |
| | certainty_metric = S[P(l2r)] | |

In some embodiments, the tumor fraction of the sample is determined (e.g., estimated) with reference to the certainty metric and the determined relationship. In some examples, the coefficients of the determined relationship are applied to the certainty metric determined from the patient sample, and the products summed to arrive at an evaluated (e.g., estimated) tumor fraction. It will be appreciated that other functions may be performed to yield a final estimated tumor fraction. For example, the estimated tumor fraction may be scaled, normalized, or otherwise adjusted from an initial or raw estimated tumor fraction measure.

In some instances, a limit-of-detection (LoD) for accurately determining tumor fraction using the TFE process may range from about 0.01% to about 5%. In some instances, the limit-of-detection for accurately determining tumor fraction may be at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 1%, at least 1.5%, at least 2%, at least 2.5%, at least 3%, at least 3.5%, at least 4%, at least 4.5%, or at least 5%. In some instances, the limit-of-detection for accurately determining tumor fraction according to the TFE process may be any value within the preceding range of values.

In some instances, determining a tumor fraction in a sample using the TFE process may provide accurate determinations of tumor fraction over a wide range of tumor DNA concentration. In some instances, the accuracy for determining the tumor fraction in a sample may range from within about ±0.2% to within about ±10% of the tumor fraction determined by a reference method for samples containing a tumor fraction ranging from about 1% to about 50%. In some instances, the accuracy for determining the tumor fraction in a sample be within about ±10%, ±9%, ±8%, ±7%, ±6%, ±5%, ±4%, ±3%, ±2%, ±1%, ±0.9%, ±0.8%, ±0.7%, ±0.6%, ±0.5%, ±0.4%, ±0.3%, or ±0.2% (or any value within this range) of the value determined by a reference method for samples comprising a tumor fraction ranging from about 1%, 1.5%, 2%, 2.5%, 5%, 10%, 15%, 20%, 25%, 30%, 35%, 40%, 45%, or 50% to about 1%, 1.5%, 2%, 2.5%, 5%, 10%, 15%, 20%, 25%, 30%, 35%, 40%, 45%, or 50% (or a tumor fraction ranging between any pair of increasing values within this range).

### (ii) Maximum Allele Frequency

In some embodiments, determining a tumor shed value comprises a maximum allele frequency (MAF) determination. The determination of MAF begins with the input of the sequencing data for the liquid biopsy sample (e.g., sequence data for variant sequences at the plurality of loci selected for analysis). In some instances, the sequencing data may be, e.g., sequencing data for cell-free DNA (cfDNA) in the sample (e.g., a liquid biopsy sample). Variant alleles for which the variant allele frequency (VAF) is greater than an upper bound for estimated tumor fraction determined using the TFE method are excluded from the determination. The upper bound determined by TFE gives a 95% confidence limit for the ctDNA fraction in a sample. Variants with VAF above this threshold are excluded to limit potential overestimations of ctDNA content due to germline variants or variants with elevated VAF due to amplification. Known germline variant sequences (e.g., variants with consensus SGZ germline status), variant sequences associated with clonal hematopoiesis of indeterminate potential (CHIP variants), sequencing artifacts, and the like, are also excluded from the determination by, e.g., comparing variant sequences against one or more sequence databases. In some instances, variant alleles having a VAF ranging from 40-60% (in this version of MSAF there is no upper bound to use as a filter, so 40-60% represents a broad range that should capture most germline variants) are also excluded. In some embodiments, variants of unknown significance (VUS) are also excluded.

The remaining variant sequences are then iteratively examined to identify variants which occur on amplified alleles (median log 2 ratio>1.0, where the log 2 ratio is the logarithm base 2 of the ratio of sample coverage and matched coverage across the targeted region for a specific allele). For these variants, base coverage and copy number are used to correct their VAF by reverse engineering what the VAF would be if the variant occurred on a non-amplified allele. The value of the highest VAF observed for all remaining coding variants is assigned as the output value for the estimated tumor fraction of the sample. The sequencing data is also examined for the presence of rearrangements. If no rearrangements are detected, the highest value of VAF is output as the final determination of tumor fraction in the sample. If rearrangements are detected, but the rearrangement VAF is less than the estimated tumor fraction based on the previously determined highest value of VAF, the highest value of VAF is kept as the final determination of tumor fraction in the sample. If rearrangements are detected, and the rearrangement VAF is greater than or equal to the estimated tumor fraction based on the previously determined highest value of VAF, the highest observed value for rearrangement VAF is output as the final determination of tumor fraction for the sample.

If the output value for the tumor fraction of the sample is zero, the original sequencing data is checked for detection of amplifications (i.e. copy number gains for one or more loci being analyzed) by the sequencing data analysis pipeline used to input the variant sequence data, and a first override is returned if amplifications have been detected. If the output value for the tumor fraction of the sample is zero, the original sequencing data is also checked for deflections in SNP allele frequencies (i.e., deflections from an expected value of 0.5), and a second override is returned if an average minor allele frequency>0.47 was observed.

In some instances, a limit-of-detection (LoD) for accurately determining tumor fraction using the determination of MAF may range from about 0.01% to about 2.5%. In some instances, the limit-of-detection for accurately determining tumor fraction may be at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 1%, at least 1.5%, at least 2%, at least 2.5%, at least 3%, at least 3.5%, at least 4%, at least 4.5%, or at least 5%. In some instances, the limit-of-detection for accurately determining tumor fraction according to the MAF method may be any value within the preceding range of values.

(iii) Composite Tumor Fraction

In some embodiments, determining a tumor shed value comprises determining a composite tumor fraction (cTF). In some embodiments, determining the cTF comprises receiving a plurality of values, each value indicative of an allele fraction at a corresponding locus within a subgenomic interval in the sample; determining a certainty metric value indicative of a dispersion of the plurality of values; determining a first estimate of the tumor fraction of the sample, the first estimate based on the certainty metric value for the sample and a predetermined relationship between one or more stored certainty metric values and one or more stored tumor fraction values; determining whether a value associated with the first estimate is greater than a first threshold, wherein if the value associated with the first estimate is greater than the first threshold, the first estimate determined as the tumor fraction of the sample; and if the value associated with the first estimate is less than or equal to the first threshold, determining a second estimate of the tumor fraction of the sample based on an allele frequency determination, wherein the second estimate is determined as the tumor fraction of the sample. In some embodiments, the first determination of somatic allele frequency and the second determination of somatic allele frequency further comprise removing variant allele frequencies from the determination that correspond to germline variants, clonal hematopoiesis of indeterminate potential (CHIP) variants, and sequencing artifact variants, prior to determining the second estimate of the tumor fraction of the sample.

In some embodiments, the cTF is a cTFv2. In some embodiments, determining a cTFv2 comprises receiving a plurality of values, each value indicative of an allele fraction at a corresponding locus within a subgenomic interval in the sample; determining a certainty metric value indicative of a dispersion of the plurality of values; determining a first estimate of the tumor fraction of the sample, the first estimate based on the certainty metric value for the sample and a predetermined relationship between one or more stored certainty metric values and one or more stored tumor fraction values; determining whether a value associated with the first estimate is greater than a first threshold, wherein if the value associated with the first estimate is greater than the first threshold, the first estimate determined as the tumor fraction of the sample; and if the value associated with the first estimate is less than or equal to the first threshold, determining a second estimate of the tumor fraction of the sample based on an allele frequency determination, wherein the second estimate is determined as the tumor fraction of the sample. In some embodiments, the first determination of somatic allele frequency and the second determination of somatic allele frequency further comprise removing variant allele frequencies from the determination that correspond to germline variants, clonal hematopoiesis of indeterminate potential (CHIP) variants, variants of unknown significance (VUS) and sequencing artifact variants, prior to determining the second estimate of the tumor fraction of the sample.

For cTF determination, sequencing data (e.g., cell-free DNA (cfDNA) sequencing data obtained using a CGP assay) representing values for, e.g., allele fraction, at a plurality of loci within a genome or subgenomic interval of a subject may be processed according to a first TFE process. In some instances, the values derived from the sequencing data may represent, e.g., a difference between an allele coverage of a locus in a tumor sample and an allele coverage of the same locus in a non-tumor sample at the plurality of loci within the genome or subgenomic interval of the subject. The estimate of tumor fraction (e.g., a circulating tumor fraction) for the sample returned by the first stage determination is compared to a first threshold. The first threshold may be, for example, a limit-of-detection (LoD) or specified confidence level for determining tumor fraction using the first stage determination. If the estimated tumor fraction returned by the first stage determination is greater than the first threshold, the estimate is output as the determined value of the tumor fraction for the sample. If the estimated tumor fraction returned by the first stage determination is less than or equal to the first threshold, a secondary process may be used to calculate tumor fraction for the sample. In some instances, the secondary method may comprise the use of, for example, a maximum allele frequency (MAF) determination to estimate tumor fraction of the sample. In some instances, the use of two complementary processes in a composite methodology for determining tumor fraction provides for more accurate determinations of tumor fraction over a larger range of DNA concentrations (e.g., circulating tumor DNA (ctDNA) concentrations).

If the estimated tumor fraction returned by the first stage determination is less than or equal to the first threshold, the sequencing data may be examined for quality control issues. For example, a quality metric may be calculated for the sequencing data and compared to a quality control threshold (e.g., a second threshold). Examples of quality control parameters that may be examined, used as a quality metric, and/or used to calculate a quality metric for the sequencing data include, but are not limited to, an average sequence coverage for the sample, a minimum average sequence coverage for the sample, an allele coverage at each of the corresponding loci in the plurality of loci, a minimum allele coverage at each of the corresponding loci in the plurality of loci, a degree of nucleic acid contamination in the sample (determined, e.g., by quantifying aberrations in SNP allele frequencies), a maximum degree of nucleic acid contamination in the sample, a number of single nucleotide polymorphism (SNP) loci within the plurality of loci examined a minimum number of single nucleotide polymorphism (SNP) loci within the plurality of loci examined, or any combination thereof. In some instances, the quality control threshold (or second threshold) may comprise a specified lower limit of the quality metric.

In some instances, determining a tumor fraction in a sample using the cTF method may provide improved accuracy in determining tumor fraction over a wider range of tumor DNA concentration. In some instances, the accuracy for determining the tumor fraction in a sample may range from within about ±0.1% to within about ±10% of the tumor fraction determined by a reference method for samples containing a tumor fraction ranging from about 0.1% to about 50%. In some instances, the accuracy for determining the tumor fraction in a sample be within about ±10%, ±9%, ±8%, ±7%, ±6%, ±5%, ±4%, ±3%, ±2%, ±1%, ±0.9%, ±0.8%, ±0.7%, ±0.6%, ±0.5%, ±0.4%, ±0.3%, ±0.2%, or ±0.1% (or any value within this range) of the value determined by a reference method for samples comprising a tumor fraction ranging from about 0.1%, 0.2%, 0.3%, 0.4%, 0.5%, 1%, 1.5%, 2%, 2.5%, 5%, 10%, 15%, 20%, 25%, 30%, 35%, 40%, 45%, or 50% to about 0.1%, 0.2%, 0.3%, 0.4%, 0.5%, 1%, 1.5%, 2%, 2.5%, 5%, 10%, 15%, 20%, 25%, 30%, 35%, 40%, 45%, or 50% (or a tumor fraction ranging between any pair of increasing values within this range).

In some instances, determining the tumor shed value comprises analysis of samples comprising a quantity of cell-free DNA (cfDNA) and/or circulating tumor DNA (ctDNA) ranging from about 25 nanograms to about 1,000 nanograms. In some instances, the quantity of cfDNA and/or ctDNA in the sample may be at least 25 nanograms, at least 50 nanograms, at least 75 nanograms, at least 100 nanograms, at least 200 nanograms, at least 300 nanograms, at least 400 nanograms, at least 500 nanograms, at least 600 nanograms, at least 700 nanograms, at least 800 nanograms, at least 900 nanograms, or at least 1,000 nanograms. In some instances, the quantity of cfDNA and/or ctDNA in the sample may be at most 1,000 nanograms, at most 900 nanograms, at most 800 nanograms, at most 700 nanograms, at most 600 nanograms, at most 500 nanograms, at most 400 nanograms, at most 300 nanograms, at most 200 nanograms, at most 100 nanograms, at most 75 nanograms, at most 50 nanograms, or at most 25 nanograms. Any of the lower and upper values described in this paragraph may be combined to form a range included within the present disclosure, for example, in some instances, the quantity of cfDNA and/or ctDNA in the sample may range from about 100 nanograms to about 700 nanograms. Those of skill in the art will recognize that quantity of cfDNA and/or ctDNA in the sample may have any value within this range, e.g., about 232 nanograms.

### B. Reference Tumor Shed Value

In some embodiments, the methods comprise comparing a tumor shed value determined for a liquid biopsy sample to a reference tumor shed value. In some embodiments, the reference tumor shed value is between 0.5% to 10.0%. In some embodiments, the reference value is 0.5%. In some embodiments, the reference tumor shed value is 1.0%. In some embodiments, the reference tumor shed value is 2.0%.

In some embodiments, the reference value reference tumor shed value significantly separates a set of individuals into two groups based on significant difference in predicted responsiveness to a first therapy, and a first therapy in combination with a second therapy. In some embodiments, the reference value reference tumor shed value significantly separates a set of individuals into two groups based on significant difference in predicted responsiveness to an immune-oncology therapy, and a IO therapy in combination with chemotherapy.

### C. Liquid Biopsy Samples

In some embodiments, the methods of the disclosure comprise determining a tumor shed value in a liquid biopsy sample from an in individual having cancer. In some embodiments, the individual is a human.

In some embodiments, the liquid biopsy sample comprises a nucleic acid, e.g., DNA, RNA, or both. In certain embodiments, the sample comprises one or more nucleic acids from a cancer. In certain embodiments, the sample further comprises one or more non-nucleic acid components from the tumor, e.g., a cell, protein, carbohydrate, or lipid. In certain embodiments, the sample further comprises one or more nucleic acids from a non-tumor cell or tissue.

In some embodiments, the liquid biopsy sample comprises one or more nucleic acids, e.g., DNA, RNA, or both, from a premalignant or malignant cell, a cell from a solid tumor, a soft tissue tumor or a metastatic lesion, a cell from a hematological cancer, a histologically normal cell, a circulating tumor cells (CTCs), or a combination thereof. In some embodiments, the liquid biopsy sample comprises one or more cells chosen from a premalignant or malignant cell, a cell from a solid tumor, a soft tissue tumor or a metastatic lesion, a cell from a hematological cancer, a histologically normal cell, a circulating tumor cell (CTC), or a combination thereof.

In some embodiments, the liquid biopsy sample comprises RNA (e.g, mRNA), DNA, circulating tumor DNA (ctDNA), cell-free DNA (cfDNA), or cell-free RNA (cfRNA) from the cancer. In some embodiments, the liquid biopsy sample comprises cell-free DNA (cfDNA). In some embodiments, cfDNA comprises DNA from healthy tissue, e.g., non-diseased cells, or tumor tissue, e.g., tumor cells. In some embodiments cfDNA from tumor tissue comprises circulating tumor DNA (ctDNA). In some embodiments, the liquid biopsy sample further comprises a non-nucleic acid component, e.g., a cell, protein, carbohydrate, or lipid, e.g., from the tumor.

In some embodiments, the liquid biopsy sample comprises blood, plasma, serum, cerebrospinal fluid, sputum, stool, urine, or saliva. In some embodiments, the liquid biopsy sample comprises blood, plasma or serum. In certain embodiments, the liquid biopsy sample comprises cerebral spinal fluid (CSF). In certain embodiments, the liquid biopsy sample comprises pleural effusion. In certain embodiments, the liquid biopsy sample comprises ascites. In certain embodiments, the liquid biopsy sample comprises urine.

In some embodiments, the sample comprises a blood sample, e.g., peripheral whole blood sample. In some embodiments, the peripheral whole blood sample is collected in, e.g., two tubes, e.g., with about 8.5 ml blood per tube. In some embodiments, the peripheral whole blood sample is collected by venipuncture, e.g., according to CLSI H3-A6. In some embodiments, the blood is immediately mixed, e.g., by gentle inversion, for, e.g., about 8-10 times. In some embodiments, inversion is performed by a complete, e.g., full, 180° turn, e.g., of the wrist. In some embodiments, the blood sample is shipped, e.g., at ambient temperature, e.g., 43-99° F. or 6-37° C. on the same day as collection. In some embodiments, the blood sample is not frozen or refrigerated. In some embodiments, the collected blood sample is kept, e.g., stored, at 43-99° F. or 6-37° C.

### (i) Isolated Nucleic Acids

In some embodiments, the methods of the disclosure further comprise isolating nucleic acids from a liquid biopsy sample described herein.

In an embodiment, the method includes isolating nucleic acids from a sample to provide an isolated nucleic acid sample. In an embodiment, the method includes isolating nucleic acids from a control to provide an isolated control nucleic acid sample. In an embodiment, a method further comprises rejecting a sample with no detectable nucleic acid.

In an embodiment, the method further comprises acquiring a value for nucleic acid yield in said liquid biopsy sample and comparing the acquired value to a reference criterion, e.g., wherein if said acquired value is less than said reference criterion, then amplifying the nucleic acid prior to library construction. In an embodiment, a method further comprises acquiring a value for the size of nucleic acid fragments in said sample and comparing the acquired value to a reference criterion, e.g., a size, e.g., average size, of at least 300, 600, or 900 bps. A parameter described herein can be adjusted or selected in response to this determination.

In some embodiments, the nucleic acids are isolated when they are partially purified or substantially purified. In some embodiments, a nucleic acid is isolated when purified away from other cellular components (e.g. proteins, carbohydrates, or lipids) or other contaminants by standard techniques.

Protocols for DNA isolation from a sample are known in the art, e.g., as provided in Example 1 of International Patent Application Publication No. WO 2012/092426. Additional methods to isolate nucleic acids (e.g., DNA) from formaldehyde- or paraformaldehyde-fixed, paraffin-embedded (FFPE) tissues are disclosed, e.g., in Cronin M. et al., (2004) Am J Pathol. 164(1):35-42; Masuda N. et al., (1999) Nucleic Acids Res. 27(22):4436-4443; Specht K. et al., (2001) Am J Pathol. 158(2):419-429, Ambion RecoverAll™ Total Nucleic Acid Isolation Protocol (Ambion, Cat. No. AM1975, September 2008), Maxwell® 16 FFPE Plus LEV DNA Purification Kit Technical Manual (Promega Literature #TM349, February 2011), E.Z.N.A.® FFPE DNA Kit Handbook (OMEGA bio-tek, Norcross, GA, product numbers D3399-00, D3399-01, and D3399-02; June 2009), and QIAamp® DNA FFPE Tissue Handbook (Qiagen, Cat. No. 37625, October 2007). RecoverAll™ Total Nucleic Acid Isolation Kit uses xylene at elevated temperatures to solubilize paraffin-embedded samples and a glass-fiber filter to capture nucleic acids. Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA. Protocols for DNA isolation from blood are disclosed, e.g., in the Maxwell® 16 LEV Blood DNA Kit and Maxwell 16 Buccal Swab LEV DNA Purification Kit Technical Manual (Promega Literature #TM333, Jan. 1, 2011).

Protocols for RNA isolation are disclosed, e.g., in the Maxwell® 16 Total RNA Purification Kit Technical Bulletin (Promega Literature #TB351, August 2009).

The isolated nucleic acids (e.g., genomic DNA) can be fragmented or sheared by practicing routine techniques. For example, genomic DNA can be fragmented by physical shearing methods, enzymatic cleavage methods, chemical cleavage methods, and other methods well known to those skilled in the art. The nucleic acid library can contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library is a portion of the genome, e.g., where the complexity of the genome is reduced by design. In some embodiments, any selected portion of the genome can be used with a method described herein. In certain embodiments, the entire exome or a subset thereof is isolated.

In certain embodiments, the method further includes isolating nucleic acids from the sample to provide a library (e.g., a nucleic acid library as described herein). In certain embodiments, the sample includes whole genomic, subgenomic fragments, or both. The isolated nucleic acids can be used to prepare nucleic acid libraries. Protocols for isolating and preparing libraries from whole genomic or subgenomic fragments are known in the art (e.g., Illumina's genomic DNA sample preparation kit). In certain embodiments, the genomic or subgenomic DNA fragment is isolated from a subject's sample (e.g., a sample described herein).

In still other embodiments, the nucleic acids used to generate the library include RNA or cDNA derived from RNA. In some embodiments, the RNA includes total cellular RNA. In other embodiments, certain abundant RNA sequences (e.g., ribosomal RNAs) have been depleted. In some embodiments, the poly(A)-tailed mRNA fraction in the total RNA preparation has been enriched. In some embodiments, the cDNA is produced by random-primed cDNA synthesis methods. In other embodiments, the cDNA synthesis is initiated at the poly(A) tail of mature mRNAs by priming by oligo(dT)-containing oligonucleotides. Methods for depletion, poly(A) enrichment, and cDNA synthesis are well known to those skilled in the art.

In other embodiments, the nucleic acids are fragmented or sheared by a physical or enzymatic method, and optionally, ligated to synthetic adapters, size-selected (e.g., by preparative gel electrophoresis) and amplified (e.g., by PCR). Alternative methods for DNA shearing are known in the art, e.g., as described in Example 4 in International Patent Application Publication No. WO 2012/092426. For example, alternative DNA shearing methods can be more automatable and/or more efficient (e.g., with degraded FFPE samples). Alternatives to DNA shearing methods can also be used to avoid a ligation step during library preparation.

In other embodiments, the isolated DNA (e.g., the genomic DNA) is fragmented or sheared. In some embodiments, the library includes less than 50% of genomic DNA, such as a subfraction of genomic DNA that is a reduced representation or a defined portion of a genome, e.g., that has been subfractionated by other means. In other embodiments, the library includes all or substantially all genomic DNA.

In other embodiments, the fragmented and adapter-ligated group of nucleic acids is used without explicit size selection or amplification prior to hybrid selection. In some embodiments, the nucleic acid is amplified by a specific or non-specific nucleic acid amplification method that is well known to those skilled in the art. In some embodiments, the nucleic acid is amplified, e.g., by a whole-genome amplification method such as random-primed strand-displacement amplification.

The methods described herein can be performed using a small amount of nucleic acids, e.g., when the amount of source DNA or RNA is limiting (e.g., even after whole-genome amplification). In one embodiment, the nucleic acid comprises less than about 5 μg, 4 μg, 3 μg, 2 μg, 1 μg, 0.8 μg, 0.7 μg, 0.6 μg, 0.5 μg, or 400 ng, 300 ng, 200 ng, 100 ng, 50 ng, 10 ng, 5 ng, 1 ng, or less of nucleic acid sample. For example, one can typically begin with 50-100 ng of genomic DNA. One can start with less, however, if one amplifies the genomic DNA (e.g., using PCR) before the hybridization step, e.g., solution hybridization. Thus it is possible, but not essential, to amplify the genomic DNA before hybridization, e.g., solution hybridization.

### D. Sequencing

In some embodiments, the methods of the disclosure comprise determining a tumor shed value for an individual.

In some embodiments, the methods of the disclosure comprise determining a tumor shed value for a liquid biopsy sample by sequencing.

In some embodiments, sequencing comprises providing a plurality of nucleic acid molecules obtained from the sample; amplifying nucleic acid molecules from the plurality of nucleic acid molecules; capturing nucleic acid molecules from the amplified nucleic acid molecules; and sequencing, by a sequencer, the captured nucleic acid molecules to obtain a plurality of sequence reads corresponding to one or more genomic loci within a subgenomic interval in the sample. In some embodiments, the plurality of nucleic acid molecules comprises a mixture of tumor nucleic acid molecules and non-tumor nucleic acid molecules.

In some embodiments, amplification of the nucleic acid molecules is performed by a polymerase chain reaction (PCR) technique, a non-PCR amplification technique, or an isothermal amplification technique.

In some embodiments, sequencing further comprises ligating one or more adapters onto one or more nucleic acid molecules from the plurality of nucleic acid molecules. In some embodiments, the adapters comprise one or more of amplification primer sequences, flow cell adapter hybridization sequences, unique molecular identifier sequences, substrate adapter sequences, or sample index sequences.

In some embodiments, nucleic acid molecules from a library are isolated, e.g., using solution hybridization, thereby providing a library catch. The library catch, or a subgroup thereof, can be sequenced. Accordingly, the methods described herein can further include analyzing the library catch. In some embodiments, the library catch is analyzed by a sequencing method, e.g., a next-generation sequencing method as described herein. In some embodiments, the method includes isolating a library catch by solution hybridization, and subjecting the library catch to nucleic acid sequencing. In certain embodiments, the library catch is re-sequenced.

In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, the one or more bait molecules each comprise a capture moiety. In some embodiments, the capture moiety is biotin.

Any method of sequencing known in the art can be used. Sequencing of nucleic acids, e.g., isolated by solution hybridization, are typically carried out using next-generation sequencing (NGS). Sequencing methods suitable for use herein are described in the art, e.g., as described in International Patent Application Publication No. WO 2012/092426. In some embodiments, sequencing is performed using a massively parallel sequencing (MPS) technique, whole genome sequencing (WGS), whole exome sequencing, targeted sequencing, direct sequencing, next-generation sequencing (NGS), or a Sanger sequencing technique.

In some embodiments, sequencing comprises detecting alterations present in the genome, whole exome or transcriptome of an individual. In some embodiments, sequencing comprises DNA and/or RNA sequencing, e.g., targeted DNA and/or RNA sequencing. In some embodiments, the sequencing comprises detection of a change (e.g., an increase or decrease) in the level of a gene or gene product, e.g., a change in expression of a gene or gene product described herein.

Sequencing can, optionally, include a step of enriching a sample for a target RNA. In other embodiments, sequencing includes a step of depleting the sample of certain high abundance RNAs, e.g., ribosomal or globin RNAs. The RNA sequencing methods can be used, alone or in combination with the DNA sequencing methods described herein. In one embodiment, sequencing includes a DNA sequencing step and an RNA sequencing step. The methods can be performed in any order. For example, the method can include confirming by RNA sequencing the expression of an alteration described herein, e.g., confirming expression of a mutation or a fusion detected by the DNA sequencing methods of the invention. In other embodiments, sequencing includes performing an RNA sequencing step, followed by a DNA sequencing step.

### E. Individuals

In some embodiments, the sample is obtained, e.g., collected, from an individual, e.g., patient, with a condition or disease, e.g., a hyperproliferative disease or a non-cancer indication. In some embodiments, the disease is a hyperproliferative disease. In some embodiments, the hyperproliferative disease is a cancer, e.g., a solid tumor or a hematological cancer. In some embodiments, the cancer is a solid tumor. In some embodiments, the cancer is a hematological cancer, e.g. a leukemia or lymphoma. In some embodiments, the sample is a liquid biopsy sample.

In some embodiments, the individual has a cancer. In some embodiments, the individual has been, or is being treated, for cancer. In some embodiments, the individual is in need of being monitored for cancer progression or regression, e.g., after being treated with a cancer therapy. In some embodiments, the individual is in need of being monitored for relapse of cancer. In some embodiments, the individual is at risk of having a cancer. In some embodiments, the individual is suspected of having cancer. In some embodiments, the individual is being tested for cancer. In some embodiments, the individual has a genetic predisposition to a cancer (e.g., having a mutation that increases his or her baseline risk for developing a cancer). In some embodiments, the individual has been exposed to an environment (e.g., radiation or chemical) that increases his or her risk for developing a cancer. In some embodiments, the individual is in need of being monitored for development of a cancer.

In certain embodiments, the liquid biopsy sample is from an individual having a cancer. Exemplary cancers include, but are not limited to, B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like. In some embodiments, the cancer is a NSCLC, colorectal cancer, cholangiocarcinoma, breast cancer, stomach cancer, melanoma, pancreatic cancer, prostate cancer, ovarian cancer, esophageal cancer, or a cancer of unknown primary.

In certain embodiments, the liquid biopsy sample from an individual having a solid tumor, a hematological cancer, or a metastatic form thereof. In certain embodiments, the sample is obtained from a subject having a cancer, or at risk of having a cancer. In certain embodiments, the liquid biopsy sample is obtained from an individual who has not received a therapy to treat a cancer, is receiving a therapy to treat a cancer, or has received a therapy to treat a cancer, as described herein.

In some embodiments, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm. Premaligancy, as used herein, refers to a tissue that is not yet malignant but is poised to become malignant.

In some embodiments, the patient has been previously treated with an anti-cancer therapy, e.g., one or more anti-cancer therapies (e.g. any of the anti-cancer therapies of the disclosure). For example, the liquid biopsy sample may be from an individual that has been treated with an anti-cancer therapy comprising one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, or a cytotoxic agent. In some embodiments, the individual has previously been treated with a chemotherapy or an immune-oncology therapy. In some embodiments, for a patient who has been previously treated with an anti-cancer therapy, a post-anti-cancer therapy sample, e.g., specimen is obtained, e.g., collected. In some embodiments, the post-anti-cancer therapy sample is a sample obtained, e.g., collected, after the completion of the targeted therapy.

In some embodiments, the patient has not been previously treated with an anti-cancer therapy.

In some embodiments, the individual is a human. In some embodiments, the individual is a non-human mammal.

### IV. Methods of Use of Tumor Fraction

Once a tumor shed value has been determined in a liquid biopsy sample from an individual having cancer, and compared to a reference tumor shed value, the disclosure provides for therapies or further analysis of a biomarker responsive to said comparison. The individual may be any of the individuals described in Section III. D. of the disclosure.

### A. Therapies

Some aspects of the disclosure provide for therapies. In some embodiments, the therapy comprises an immune-oncology (IO) therapy, or an IO therapy in combination with a chemotherapy. In some embodiments, the therapy comprises a targeted therapy. In some embodiments, the therapy comprises an anti-cancer therapy.

### (i) Immuno-Oncology Therapies

Certain aspects of the present disclosure relate to immuno-oncology (IO) therapies. In some embodiments the IO therapy comprises an immune checkpoint inhibitor (ICI).

As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCNI, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSFi4, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments according to any of the embodiments described herein, the immune checkpoint inhibitor comprises a PD-1 antagonist/inhibitor or a PD-L1 antagonist/inhibitor.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.I. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist/inhibitor is a molecule that inhibits the binding of PD-1 to its ligand binding partners. In a specific embodiment, the PD-1 ligand binding partners are PD-L1 and/or PD-L2. In another instance, a PD-L1 binding antagonist/inhibitor is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is MDX-1 106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANBO11; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (1B1308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MED14736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist/inhibitor of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MED14736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MED14736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the immune checkpoint inhibitor is monovalent and/or monospecific. In some embodiments, the immune checkpoint inhibitor is multivalent and/or multispecific.

In some embodiments, the immune checkpoint inhibitor may be administered in combination with an immunoregulatory molecule or a cytokine. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3a (Lax), MIP-30, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is a first line immune checkpoint inhibitor. In some embodiments, the immune checkpoint inhibitor is a second line immune checkpoint inhibitor. In some embodiments, an immune checkpoint inhibitor is administered in combination with one or more additional anti-cancer therapies or treatments.

In some embodiments, the methods of the disclosure further comprise treating an individual with the 10 therapy. In some embodiments, an 10 therapy is administered as a monotherapy. In some embodiments, the 10 therapy comprises one or multiple 10 agents.

In some embodiments, the individual is treated with an 10 therapy in combination with a second therapy. In some embodiments, the individual with the 10 therapy in combination with a chemotherapy. In some embodiments, the 10 therapy and the chemotherapy are administered concurrently or sequentially.

### (ii) Chemotherapies

Certain aspects of the present disclosure relate to chemotherapies.

In some embodiments, the methods provided herein comprise administering to an individual a chemotherapy, e.g., in combination with another anti-cancer therapy of the disclosure, such as an 10 therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, farnesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure, such as an IO therapy, are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

(iii) Targeted Therapies

Certain aspects of the disclosure provide for targeted therapies.

In some embodiments, the targeted therapy is an TMB-targeted therapy. In some embodiments, the TMB-targeted therapy comprises an immune checkpoint inhibitor. In some embodiments, the immune checkpoint inhibitor is an anti-PD1 therapy or an anti-PD-L1 therapy. In some embodiments, the anti-PD-1 therapy comprises one or more of nivolumab, pembrolizumab, cemiplimab, or dostarlimab. In some embodiments, the anti-PD-L1 therapy comprises one or more of atezolizumab, avelumab, or durvalumab. In some embodiments, the TMB-targeted therapy is administered to an individual having a TMB high score.

In some embodiments, the targeted therapy is a MSI-high-targeted therapy. In some embodiments, the MSI-high-targeted therapy comprises an immune checkpoint inhibitor. In some embodiments, the immune checkpoint inhibitor is an anti-PD1 therapy, an anti-PD-L1 therapy, or an anti-CTLA-4 therapy. In some embodiments, the anti-PD-1 therapy comprises one or more of nivolumab, pembrolizumab, cemiplimab, or dostarlimab. In some embodiments, the anti-PD-1 therapy comprises one or more of nivolumab, pembrolizumab, cemiplimab, or dostarlimab. In some embodiments, the anti-CTLA-4 therapy comprises ipilimumab In some embodiments, the anti-PD-L1 therapy comprises one or more of atezolizumab, avelumab, or durvalumab. In some embodiments, the MSI-high-targeted therapy is administered to an individual having a MSI-high score.

In some embodiments, the targeted therapy is an HRD-positive targeted therapy.

Treatments that are effective in a HRD positive tumor and may be used as a HRD-positive targeted therapy can include one or more PARP inhibitors and/or one or more platinum-based agents. PARP inhibitors may include, but are not limited to, veliparib, olaparib, talazoparib, iniparib, rucaparib, and niraparib. PARP inhibitors are described in Murphy and Muggia, PARP inhibitors: clinical development, emerging differences, and the current therapeutic issues, Cancer Drug Resist 2019; 2:665-79. Platinum-based agents may include, but are not limited to, cisplatin, oxaliplatin, and carboplatin. Platinum-based drugs are described in Rottenberg et al., The rediscovery of platinum-based cancer therapy, Nat. Rev. Cancer 2021 January; 21(1):37-50.

In some embodiments, the HRD-positive targeted therapy is selected from the group consisting of a platinum-based drug and a PARP inhibitor, or any combination thereof. In some embodiments, the PARP inhibitor is olaparib, niraparib, or rucaparib. In some embodiments, the HRD-positive targeted therapy is administered to an individual having an HRD-positive status.

### (iv) Anti-Cancer Therapies

Certain aspects of the disclosure provide for anti-cancer therapies.

In some embodiments, the anti-cancer therapy comprises a kinase inhibitor. In some embodiments, the methods provided herein comprise administering to the individual a kinase inhibitor, e.g., in combination with another anti-cancer therapy such as an immune checkpoint inhibitor. Examples of kinase inhibitors include those that target one or more receptor tyrosine kinases, e.g., BCR-ABL, B-Raf, EGFR, HER-2/ErbB2, IGF-IR, PDGFR-α, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy such as an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy such as an immune checkpoint inhibitor. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy such as an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy such as an immune checkpoint inhibitor. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy such as an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy such as an immune checkpoint inhibitor. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy such as an immune checkpoint inhibitor. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some embodiments, the anti-cancer therapy comprises a cancer immunotherapy, such as a cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a cancer vaccine, cell-based therapy, T cell receptor (TCR)-based therapy, adjuvant immunotherapy, cytokine immunotherapy, and oncolytic virus therapy, e.g., in combination with another anti-cancer therapy such as an immune checkpoint inhibitor. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA or RNA that encodes a neoantigen. In some embodiments, the cancer vaccine comprises a polynucleotide that encodes a neoantigen. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-lh68/GLV-lh153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or BSR. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration. In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

### B. Biomarkers

In some embodiments of the methods of the disclosure, the methods comprise further analyzing a biomarker if a tumor shed value in a liquid biopsy sample is equal or higher to a reference tumor shed value. In some embodiments, the biomarker is a tumor mutational burden (TMB) score, a homologous recombination deficiency (HRD) score, or a microsatellite instability (MSI) status. In some embodiments, the biomarker comprises one or more alterations in one or more genes.

### (i) Tumor Mutation Burden

Some aspects of the disclosure provide for further analysis of a tumor mutation burden (TMB) score. In some embodiments, the level of TMB corresponds to a TMB score. In some embodiments, the TMB is blood TMB (bTMB).

As used herein, the terms “blood tumor mutational burden score,” “blood tumor mutation burden score,” and “bTMB score,” each of which may be used interchangeably, refer to a numerical value that reflects the number of somatic mutations detected in a blood sample (e.g., a whole blood sample, a plasma sample, a serum sample, or a combination thereof) obtained from an individual (e.g., an individual at risk of or having a cancer). The bTMB score can be measured, for example, on a whole genome or exome basis, or on the basis of a subset of the genome or exome (e.g., a predetermined set of genes). In certain embodiments, a bTMB score can be measured based on intergenic sequences. In some embodiments, the bTMB score measured on the basis of a subset of genome or exome can be extrapolated to determine a whole genome or exome bTMB score. In certain embodiments, the predetermined set of genes does not comprise the entire genome or the entire exome. In other embodiments, the set of subgenomic intervals does not comprise the entire genome or the entire exome. In some embodiments, the predetermined set of genes comprise a plurality of genes, which in mutant form, are associated with an effect on cell division, growth or survival, or are associated with cancer. In some embodiments, the predetermined set of genes comprise at least about 50 or more, about 100 or more, about 150 or more, about 200 or more, about 250 or more, about 300 or more, about 350 or more, about 400 or more, about 450 or more, or about 500 or more genes. In some embodiments, the pre-determined set of genes covers about 1 Mb (e.g., about 1.1 Mb, e.g., about 1.125 Mb). In some embodiments, the bTMB score is determined from measuring the number of somatic mutations in cell-free DNA (cfDNA) in a sample. In some embodiments, the bTMB score is determined from measuring the number of somatic mutations in circulating tumor DNA (ctDNA) in a sample. In some embodiments, the number of somatic mutations is the number of single nucleotide variants (SNVs) counted or a sum of the number of SNVs and the number of indel mutations counted. In some embodiments, the bTMB score refers to the number of accumulated somatic mutations in a tumor.

In some embodiments, tumor mutational burden (e.g. bTMB) is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety. In some embodiments, tumor mutational burden is measured according to the methods described in Montesion, M., et al., Cancer Discovery (2021) 11(2):282-92.

In some embodiments, tumor mutational burden is assessed based on the number of non-driver somatic coding mutations/megabase (mut/Mb) of genome sequenced.

In some embodiments, tumor mutational burden is measured in the sample by whole exome sequencing. In some embodiments, tumor mutational burden is measured in the sample using next-generation sequencing. In some embodiments, tumor mutational burden is measured in the sample using whole genome sequencing. In some embodiments, tumor mutational burden is measured in the sample by gene-targeted sequencing. In some embodiments, tumor mutational burden is measured on between about 0.8 Mb and about 1.3 Mb of sequenced DNA. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, about 1.1 Mb, about 1.2 Mb, or about 1.3 Mb of sequenced DNA. In some embodiments, tumor mutational burden is measured on about 0.8 Mb of sequenced DNA. In some embodiments, tumor mutational burden is measured on between about 0.83 Mb and about 1.14 Mb of sequenced DNA. In some embodiments, tumor mutational burden is measured on up to about 1.24 Mb of sequenced DNA. In some embodiments, tumor mutational burden is measured on up to about 1.1 Mb of sequenced DNA. In some embodiments, tumor mutational burden is measured on about 0.79 Mb of sequenced DNA.

In some embodiments, the TMB score is less than about 10 mut/Mb, e.g., any of about 9.9 mut/Mb, about 9.8 mut/Mb, about 9.6 mut/Mb, about 9.4 mut/Mb, about 9.2 mut/Mb, about 9 mut/Mb, about 8.8 mut/Mb, about 8.6 mut/Mb, about 8.4 mut/Mb, about 8.2 mut/Mb, about 8 mut/Mb, about 7.8 mut/Mb, about 7.6 mut/Mb, about 7.4 mut/Mb, about 7.2 mut/Mb, about 7 mut/Mb, about 6.8 mut/Mb, about 6.6 mut/Mb, about 6.4 mut/Mb, about 6.2 mut/Mb, about 6 mut/Mb, about 5.8 mut/Mb, about 5.6 mut/Mb, about 5.4 mut/Mb, about 5.2 mut/Mb, about 5 mut/Mb, about 4.8 mut/Mb, about 4.6 mut/Mb, about 4.4 mut/Mb, about 4.2 mut/Mb, about 4 mut/Mb, about 3.8 mut/Mb, about 3.6 mut/Mb, about 3.4 mut/Mb, about 3.2 mut/Mb, about 3 mut/Mb, about 2.8 mut/Mb, about 2.6 mut/Mb, about 2.4 mut/Mb, about 2.2 mut/Mb, about 2 mut/Mb, about 1.8 mut/Mb, about 1.6 mut/Mb, about 1.4 mut/Mb, about 1.2 mut/Mb, about 1 mut/Mb, about 0.8 mut/Mb, about 0.6 mut/Mb, about 0.4 mut/Mb, about 0.2 mut/Mb, or less.

In some embodiments, the TMB score is a high tumor mutational burden score, e.g., of at least about 10 mut/Mb. In some embodiments, the TMB score is at least about 10 mut/Mb. In some embodiments, the TMB score is at least about 20 mut/Mb. In some embodiments, the TMB score is between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the TMB score is between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, the TMB score is at least about 100 mut/Mb, at least about 110 mut/Mb, at least about 120 mut/Mb, at least about 130 mut/Mb, at least about 140 mut/Mb, at least about 150 mut/Mb, or more.

In some embodiments, the TMB score is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb. In some embodiments, the TMB is at least about 5 mutations/Mb, at least about 10 mutations/Mb, at least about 12 mutations/Mb, at least about 16 mutations/Mb, at least about 20 mutations/Mb, or at least about 30 mutations/Mb. In some embodiments, the TMB score is determined based on between about 100 kb to about 10 Mb. In some embodiments, the TMB score is determined based on between about 0.8 Mb to about 1.1 Mb.

In some embodiments, measuring tumor mutational burden comprises assessing mutations in a liquid biopsy sample derived from an individual having cancer. In some embodiments, measuring tumor mutational burden comprises assessing mutations in a liquid biopsy sample derived from a cancer in an individual and in a matched normal sample, e.g., a sample from the individual derived from a tissue or other source that is free of the cancer.

In some embodiments, tumor mutational burden is obtained from a plurality of sequence reads, e.g., a plurality of sequence reads obtained by sequencing nucleic acids corresponding to at least a portion of a genome (such as from an enriched or unenriched sample). In some embodiments, tumor mutational burden is determined based on the number of non-driver somatic coding mutations per megabase of genome sequenced.

### (ii) Microsatellite Instability

Some aspects of the disclosure provide for further analysis of a microsatellite instability (MSI) status.

Microsatellite instability may be assessed using any suitable method known in the art. For example, microsatellite instability may be measured using next generation sequencing (see, e.g., Hempelmann et al., J Immunother Cancer (2018) 6(1):29), Fluorescent multiplex PCR and capillary electrophoresis (see, e.g., Arulananda et al., J Thorac Oncol (2018) 13(10):1588-94), immunohistochemistry (see, e.g., Cheah et al., Malays J Pathol (2019) 41(2):91-100), or single-molecule molecular inversion probes (smMIPs, see, e.g., Waalkes et al., Clin Chem (2018) 64(6):950-8). In some embodiments, microsatellite instability is assessed based on DNA sequencing (e.g., next generation sequencing) of up to about 114 loci. In some embodiments, microsatellite instability is assessed based on DNA sequencing (e.g., next generation sequencing) of intronic homopolymer repeat loci for length variability. In some embodiments, microsatellite instability is assessed based on DNA sequencing (e.g., next generation sequencing) about 114 intronic homopolymer repeat loci for length variability. In some embodiments, microsatellite instability status (e.g., microsatellite instability high) is defined as described in Trabucco et al., J Mol Diagn. 2019 November; 21(6):1053-1066.

(iii) Homology-Deficient Recombination

Some aspects of the disclosure provide for further analysis of a homology-deficient recombination (HRD) score. In some embodiments, the HRD score is an HRD-positive score.

In some embodiments, further analyzing an HRD score comprises using a HRD classification model. A trained HRD classification model may be is configured to classify a tumor as HRD-positive (or likely HRD positive) or HRD-negative (or likely HRD negative). The HRD classification model is trained using HRD positive data comprising, for each HRD-positive tumor in a plurality of HRD-positive tumors, one or more data features (such as one or more copy number features and/or one or more short variant features, among other possible features) associated with the HRD-positive tumors and a HRD-positive label. The HRD classification model is further trained using HRD negative data comprising, for each HRD-negative tumor in a plurality of HRD-negative tumors, one or more data features (such as one or more copy number features and/or one or more short variant features, among other possible features) associated with the HRD-negative tumors and a HRD-negative label. Test data comprising one or more data features (such as one or more copy number features and/or one or more short variant features, among other possible features) associated with a genome of a tumor in a subject is input into the trained HRD classification model, which then classifies the tumor as HRD-positive (or likely HRD positive) or HRD-negative (or likely HRD negative) based on the test data.

The HRD classifier may be a probabilistic classifier, such as a gradient boosting model. The probabilistic classifier can be configured to compute a probability that the tumor is HRD positive or HRD negative, such as by outputting a HRD positive likelihood score or a HRD negative likelihood score. Based on the probability or probabilities outputted from the HRD classification model, the tumor can be called as being HRD positive or HRD negative. Optionally, the tumor may be called as ambiguous, for example if neither the probability that the tumor is HRD positive nor that the probability that the tumor is HRD negative is above a predetermined probability threshold. The HRD positive data and the HRD negative data can include the copy number features and/or the short variant features described herein.

The HRD negative data may comprise genomes with wild-type alleles (i.e., alleles not associated with HRD) at certain HRD-associated genes. For example, in some embodiments, the HRD negative data comprises data associated with genomes with wild-type alleles at one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD negative data comprises promoter methylation data of one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD negative data comprises RNA expression data of one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD negative data comprises data associated with genomes associated with tumors that were found to be resistant to platinum-based drugs (e.g., chemotherapy) and/or PARP inhibitors. In some embodiments, the HRD negative data comprises data associated with genomes associated with tumors previously classified as HRD negative. In some embodiments, the HRD negative data is, at least in part, derived from a consensus human genome sequence, or a portion thereof.

The HRD positive data may comprise data associated with genomes with HRD-associated alleles at certain HRD-associated genes. For example, in some embodiments, the HRD positive data comprises data associated with genomes with mutations at one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L, particularly biallelic mutations thereof. In some embodiments, the HRD positive data comprises promoter methylation data of one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD positive data comprises RNA expression data of one or more of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. In some embodiments, the HRD positive data comprises data associated with genomes associated with tumors that were found to be sensitive to platinum-based drugs and/or PARP inhibitors. In some embodiments, the HRD positive data comprises data associated with genomes associated with tumors previously classified as HRD positive. In some embodiments, the HRD positive data comprises data associated with tumors having biallelic BRCA1 and BRCA2 mutations associated with HRD.

The HRD positive data may be balanced with the HRD negative data. For example, in an unbalanced training dataset, the number of HRD positive training tumors may outnumber the number of HRD negative tumors (or vice versa). Balancing the data ensures the model has a sufficient number of each label to avoid biasing to one label. When balanced, the number of HRD positive tumors or the number of HRD negative tumors are adjusted so that the ratio between them is at a desired level (such as approximately 1:1 or any other desired ratio). Using the balanced dataset, the HRD classifier may be trained and then tested against a test dataset comprising HRD positive tumors and HRD negative tumors.

The tumors used to train the HRD classifier each comprise an HRD positive label or a HRD negative label. Any suitable methodology may be used to computationally label (e.g., apply a metadata tag to) the tumors as HRD positive or HRD negative. An HRD positive label may be assigned by the presence of alterations in one of the HRD-associated genes, such as one of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L, particularly biallelic alterations thereof. Mutations in one or both of BRCA1 and BRCA2 are especially indicative of HRD positivity, especially biallelic BRCA1/BRCA2 mutations. Tumors may also be labeled as HRD positive based on clinical history. For example, if a tumor was sensitive to a PARP inhibitor or a platinum-based drug regimen, then the tumor is more likely to be HRD positive. An HRD negative label may be assigned based on the absence of alterations in one of the HRD-associated genes, such as one of a gene associated with HRD, including, but not limited to, BRCA1, BRCA2, ATM, BARD1, BRIP1, CDK12, CHEK1, CHEK2, FANCL, PALB2, RAD51B, RAD51C, RAD51D, and/or RAD45L. Mutations in HRD-associated genes may be detected by comparison of the gene sequence with a reference genome, such as a consensus human genome sequence such as hg19. Likewise, tumors may also be labeled as HRD negative based on clinical history. For example, if a tumor was resistant to a PARP inhibitor or a platinum-based drug regimen, then the tumor is more likely to be HRD negative. This is especially true if the tumor was treatment naïve prior to treatment with the PARP inhibitor or platinum-based drug regimen, since HRD positive tumors may develop resistance to these drugs after rounds of treatment. Although each tumor may comprise an HRD positive or HRD negative label, this label does not require absolute certainty that a tumor is HRD positive or HRD negative. Instead, given a robust training dataset comprising numerous HRD positive tumors and numerous HRD negative tumors, and by avoiding overfitting of these data as is known in the art, the contributions of false positives and false negatives are averaged out in the model. Further, the use of a larger training dataset, particularly a balanced training dataset and a dataset having well-defined positive and negative labels (such as by using validated consensus genomes for HRD-negative labels; and by using validated biallelic BRCA1/2 mutants or validated, well-characterized BRCAness samples for HRD-positive labels), allows the model to properly assess the nuanced differences between HRD-negative phenotypes and those exhibiting HRD scarring (i.e., HRD-positive phenotypes).

The HRD classifier model may classify the tumor of the cancer as HRD positive or HRD negative. In some embodiments, the HRD classifier model may classify the tumor as likely HRD positive, likely HRD negative, or ambiguous. For example, the HRD classifier model may classify the tumor as ambiguous if it cannot classify the tumor as likely HRD positive or likely HRD negative with sufficiently high confidence or probability. The confidence or probability threshold may be set by the user as desired, given the tolerance for inaccurate classification. In one example, the user may set the HRD-positive likelihood score threshold at 0.8 and the HRD-negative likelihood score threshold at 0.2. If the HRD-positive likelihood score is below 0.8 and/or if the HRD-negative likelihood score is above 0.2, then the HRD model may not classify the tumor as HRD positive, and would either classify the tumor as HRD negative (depending on how low the HRD-positive likelihood score is and how high the HRD-negative likelihood score is) or ambiguous.

In some embodiments, the HRD classifier outputs a likelihood score that the tumor is HRD positive. In some embodiments, the HRD classifier outputs a likelihood score that the tumor is HRD negative. The HRD classifier may be configured to output either or both of an HRD positive likelihood score and an HRD negative likelihood score. The HRD classifier may also be configured to output a ratio of the HRD positive likelihood score to the HRD negative likelihood score and/or a ratio of the HRD negative likelihood score to the HRD positive likelihood score. The likelihood scores may be expressed as a value from 0.0 (indicating a certainty that the tumor is not HRD positive or HRD negative) to 1.0 (indicating a certainty that the tumor is HRD positive or HRD negative). For example, the trained HRD classifier may receive test sample data comprising a plurality of data features associated with a tumor of a cancer in a subject and output an HRD positive likelihood score of 0.8 and an HRD negative likelihood score of 0.15. The HRD classifier may be configured to call the tumor as HRD positive or HRD negative based upon the likelihood score or scores. In the preceding example, based on the HRD positive likelihood score 0.8 and the HRD negative likelihood score of 0.15, the HRD classifier may call the tumor as HRD positive. In some embodiments, the HRD classifier will call the tumor as HRD positive if the HRD positive likelihood score is at least 0.6, such as at least 0.65, at least 0.70, at least 0.75, at least 0.80, at least 0.85, at least 0.90, at least 0.95, or at least 0.99. In some embodiments, the HRD classifier will call the tumor as HRD negative if the HRD negative likelihood score is at least 0.6, such as at least 0.65, at least 0.70, at least 0.75, at least 0.80, at least 0.85, at least 0.90, at least 0.95, or at least 0.99. In some embodiments, the HRD classifier will call the tumor as HRD positive if the HRD negative likelihood score is less than 0.5, such as less than 0.45, less than 0.40, less than 0.35, less than 0.30, less than 0.30, less than 0.25, less than 0.20, less than 0.15, less than 0.10, or less than 0.05. In some embodiments, the HRD classifier will call the tumor as HRD negative if the HRD positive likelihood score is less than 0.5, such as less than 0.45, less than 0.40, less than 0.35, less than 0.30, less than 0.30, less than 0.25, less than 0.20, less than 0.15, less than 0.10, or less than 0.05. In some embodiments, the HRD classifier will call the tumor as HRD positive if the HRD positive likelihood score is above a certain threshold (such as at least 0.80) and the HRD negative likelihood score is below a certain threshold (such as less than 0.25). In some embodiments, the HRD classifier will call the tumor as HRD negative if the HRD negative likelihood score is above a certain threshold (such as at least 0.80) and the HRD positive likelihood score is below a certain threshold (such as less than 0.25). In some embodiments, the HRD classifier will call the tumor as ambiguous if the HRD positive likelihood score is below a certain threshold and the HRD negative likelihood score is below threshold, or if the absolute values of the likelihood scores are within a threshold percent similarity.

### (iv) Gene Alterations

Some aspects of the disclosure provide for further analysis of one or more alterations in one or more genes.

In some embodiments, the one or more genes are ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, C11orf30, C17orf39, CALR, CARD11, CASP8, CBFB, CBL, CCND1, CCND2, CCND3, CCNE1, CD22, CD70, CD74, CD79A, CD79B, CD274, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A, KMT2D, KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NSD3, NT5C2, NTRK1 NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK. TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WTI, XPO1, XRCC2, ZNF217, and ZNF703, or any combination thereof. In some embodiments, the one or more alterations are in PIK3CA. In some embodiments, the one or more alterations comprise a base substitution, an insertion/deletion (indel), a copy number alteration, or a genomic rearrangement.

In some embodiments, further analyzing one or more alterations in one or more genes comprises detecting the presence of one or more alterations in the one or more genes. Methods for detecting gene alterations are known in the art. For example, an alteration may be detected by sequencing part or all of a gene by next-generation or other sequencing of DNA, RNA, or cDNA. In some embodiments, an alteration may be detected by PCR amplification of DNA, RNA, or cDNA. In some embodiments, an alteration may be detected by in situ hybridization using one or more polynucleotides that hybridize to a locus involved in a rearrangement or fusion and/or a corresponding fusion partner gene locus described herein, e.g., using fluorescence in situ hybridization (FISH). In some embodiments, an alteration may be detected in a cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Exemplary and non-limiting methods for detecting one or more alterations in one or more genes are provided below.

Detection of one or more alterations in one or more genes is performed using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety. In some embodiments, one or more alterations in one or more genes are detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS). In some embodiments, the sequencing comprises RNA-sequencing (RNA-seq). In some embodiments, the amplification-based assay comprises a polymerase chain reaction (PCR) amplification technique, a non-PCR amplification technique, or an isothermal amplification technique. In some embodiments, the amplification-based assay comprises a reverse transcription PCR (RT-PCR), a quantitative real-time PCR (qPCR), or a reverse transcription quantitative real-time PCR (RT-qPCR) assay. In some embodiments, the amplification-based assay comprises an RT-PCR assay.

In some embodiments, the one or more alterations in one or more genes are detected using an in situ hybridization method, such as a fluorescence in situ hybridization (FISH) method.

In some embodiments, FISH analysis is used to detect the one or more alterations in one or more genes. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion or rearrangement, e.g., at one or more exons and or introns of the gene, are utilized.

In some embodiments, the one or more alterations in one or more genes are detected using an array-based method, such as array-based comparative genomic hybridization (CGH) methods. In array-based CGH methods, a first sample of nucleic acids (e.g., from a sample, such as from a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor, or a tissue or liquid biopsy) is labeled and hybridized to a second array (with identical content) and absolute signals are read. Copy number differences are calculated based on absolute signals from the two arrays.

In some embodiments, the one or more alterations in one or more genes are detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers, e.g., such as one or more oligonucleotides or primers provided herein. The presence of one or more alterations in one or more genes in a sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, the one or more alterations in one or more genes are detected using hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference. In some embodiments, the one or more alterations in one or more genes are detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 105 molecules may be sequenced simultaneously). Next generation sequencing methods suitable for use according to the methods provided herein are known in the art and include, without limitation, massively parallel short-read sequencing, template based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect one or more alterations in one or more genes include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect one or more alterations in one or more genes are include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

### V. Exemplary Embodiments

The following embodiments are exemplary and are not intended to limit the scope of the invention.

Embodiment 1. A method for identifying an individual having a cancer for treatment with an immuno-oncology (IO) therapy and chemotherapy combination comprising (a) determining a tumor shed value for a liquid biopsy sample obtained from the individual, and (b) identifying the individual for treatment with the IO therapy and chemotherapy combination if the tumor shed value in the liquid biopsy sample is equal to or higher than a reference tumor shed value.

Embodiment 2. A method of treating an individual having a cancer with an immuno-oncology (IO) therapy and chemotherapy combination comprising (a) determining a tumor shed value for a liquid biopsy sample obtained from the individual, and (b) treating the individual with the IO therapy and chemotherapy combination if the tumor shed value in the liquid biopsy sample is equal to or higher than a reference tumor shed value.

Embodiment 3. A method of selecting a treatment for an individual having a cancer, the method comprising determining a tumor shed value for a liquid biopsy sample obtained from the individual, wherein a tumor shed value in the liquid biopsy sample that is equal to or higher than a reference tumor shed value identifies the individual as one who may benefit from treatment with an immuno-oncology (IO) therapy and chemotherapy combination.

Embodiment 4. A method of identifying one or more treatment options for an individual having a cancer, the method comprising:
(a) determining a tumor shed value for a liquid biopsy sample obtained from the individual, and
(b) generating a report comprising one or more treatment options identified for the individual based at least in part on the tumor shed value for the liquid biopsy sample, wherein a tumor shed value in the liquid biopsy sample that is equal to or higher than a reference tumor shed value identifies the individual as one who may benefit from treatment with an immuno-oncology (IO) therapy and chemotherapy combination.

Embodiment 5. A method of predicting survival of an individual having a cancer, comprising acquiring knowledge of a tumor shed value for a liquid biopsy sample obtained from the individual, wherein the if tumor shed value for the liquid biopsy sample obtained from the individual is equal to or higher than a reference tumor shed value, and wherein responsive to the acquisition of said knowledge, the individual is predicted to have longer survival when treated with an immuno-oncology (IO) therapy and chemotherapy combination, as compared to treatment with an immuno-oncology (IO) therapy without chemotherapy.

Embodiment 6. A method of monitoring, evaluating, or screening an individual having a cancer, comprising acquiring knowledge of a tumor shed value for a liquid biopsy sample obtained from the individual, wherein if the tumor shed value for the liquid biopsy sample obtained from the individual is equal to or higher than a reference tumor shed value, and wherein responsive to the acquisition of said knowledge, the individual is predicted to have longer survival when treated with an immuno-oncology (IO) therapy in combination with chemotherapy, as compared to treatment with an immuno-oncology (IO) therapy without chemotherapy.

Embodiment 7. A method for identifying an individual having a cancer for treatment with an immuno-oncology (IO) therapy comprising (a) determining a tumor shed value for a liquid biopsy sample obtained from the individual, and (b) identifying the individual for treatment with the IO therapy if the tumor shed value in the liquid biopsy sample is lower than a reference tumor shed value.

Embodiment 8. A method of treating an individual having a cancer with an immuno-oncology (IO) therapy comprising (a) determining a tumor shed value for a liquid biopsy sample obtained from the individual, and (b) treating the individual with the IO if the tumor shed value in the liquid biopsy sample is lower than a reference tumor shed value.

Embodiment 9. A method of selecting a treatment for an individual having a cancer, the method comprising determining a tumor shed value for a liquid biopsy sample obtained from the individual, wherein a tumor shed value in the liquid biopsy sample that is lower than a reference tumor shed value identifies the individual as one who may benefit from treatment with an immuno-oncology (IO) therapy.

Embodiment 10. A method of identifying one or more treatment options for an individual having a cancer, the method comprising:
(a) determining a tumor shed value for a liquid biopsy sample obtained from the individual, and
(b) generating a report comprising one or more treatment options identified for the individual based at least in part on the tumor shed value for the liquid biopsy sample, wherein a tumor shed value in the liquid biopsy sample that is lower than a reference tumor shed value identifies the individual as one who may benefit from treatment with an immuno-oncology (IO) therapy.

Embodiment 11. A method of predicting survival of an individual having a cancer, comprising acquiring knowledge of a tumor shed value for a liquid biopsy sample obtained from the individual, wherein the if tumor shed value for the liquid biopsy sample obtained from the individual is lower than a reference tumor shed value, and wherein responsive to the acquisition of said knowledge, the individual is predicted to have longer survival when treated with an immuno-oncology (IO) therapy, as compared to treatment without immuno-oncology (IO) therapy.

Embodiment 12. A method of monitoring, evaluating, or screening an individual having a cancer, comprising acquiring knowledge of a tumor shed value for a liquid biopsy sample obtained from the individual, wherein if the tumor shed value for the liquid biopsy sample obtained from the individual is lower than a reference tumor shed value, and wherein responsive to the acquisition of said knowledge, the individual is predicted to have longer survival when treated with an immuno-oncology (IO) therapy, as compared to treatment without an immuno-oncology (IO) therapy.

Embodiment 13. A method of stratifying an individual with a cancer for treatment with a therapy comprising determining a tumor shed value for a liquid biopsy sample obtained from the individual, and
(a) if the tumor shed value is equal to or higher than a reference tumor shed value, identifying the individual as a candidate for receiving an IO therapy in combination with chemotherapy; or
(b) if the tumor shed value is less than the reference tumor shed value, identifying the individual as a candidate for receiving an immuno-oncology (IO) therapy without chemotherapy.

Embodiment 14. A method for identifying an individual having a cancer for treatment with a first therapy and a second therapy comprising (a) determining a tumor shed value for a liquid biopsy sample obtained from the individual, and (b) identifying the individual for treatment with the first therapy and the second therapy if the tumor shed value in the liquid biopsy sample is equal to or greater than a reference tumor shed value.

Embodiment 15. A method of treating an individual having a cancer with a first therapy and a second therapy comprising (a) determining a tumor shed value for a liquid biopsy sample obtained from the individual, and (b) treating the individual with the first therapy and the second therapy if the tumor shed value in the liquid biopsy sample is equal to or greater than a reference tumor shed value.

Embodiment 16. A method of selecting a treatment for an individual having a cancer, the method comprising determining a tumor shed value for a liquid biopsy sample obtained from the individual, wherein a tumor shed value in the liquid biopsy sample that is equal to or greater than a reference tumor shed value identifies the individual as one who may benefit from treatment with a first therapy and a second therapy.

Embodiment 17. A method of identifying one or more treatment options for an individual having a cancer, the method comprising:
(a) determining a tumor shed value for a liquid biopsy sample obtained from the individual, and
(b) generating a report comprising one or more treatment options identified for the individual based at least in part on the tumor shed value for the liquid biopsy sample, wherein a tumor shed value in the liquid biopsy sample that is equal to or greater than a reference tumor shed value identifies the individual as one who may benefit from treatment with a first therapy and a second therapy.

Embodiment 18. A method of predicting survival of an individual having a cancer, comprising acquiring knowledge of a tumor shed value for a liquid biopsy sample obtained from the individual, wherein the tumor shed value for the liquid biopsy sample obtained from the individual is equal to or greater than a reference tumor shed value, and wherein responsive to the acquisition of said knowledge, the individual is predicted to have longer survival when treated with a first therapy and a second therapy, as compared to treatment with the first therapy without the second therapy.

Embodiment 19. A method of monitoring, evaluating, or screening an individual having a cancer, comprising acquiring knowledge of a tumor shed value for a liquid biopsy sample obtained from the individual, wherein the tumor shed value for the liquid biopsy sample obtained from the individual is equal to or greater than a reference tumor shed value, and wherein responsive to the acquisition of said knowledge, the individual is predicted to have longer survival when treated with a first therapy and a second therapy, as compared to treatment the first therapy without the second therapy.

Embodiment 20. A method of stratifying an individual with a cancer for treatment with a first therapy and a second therapy comprising determining a tumor shed value for a liquid biopsy sample obtained from the individual, and
(a) if the tumor shed value is equal to or greater than a reference tumor shed value, identifying the individual as a candidate for receiving a first therapy and a second therapy; or
(b) if the tumor shed value is less than a reference tumor shed value, identifying the individual as a candidate for receiving the first therapy without the second therapy.

Embodiment 21. The method of any one of embodiments 14-20, wherein the first therapy is an immuno-oncology (IO) therapy.

Embodiment 22. The method of any one of embodiments 14-21, wherein the second therapy is a chemotherapy.

Embodiment 23. A method of assessing a biomarker in a liquid biopsy sample from an individual having cancer, the method comprising determining a tumor shed value for the individual, and wherein the tumor shed value is equal to or greater than a reference tumor shed value, further analyzing the biomarker.

Embodiment 24. The method of embodiment 23, wherein the biomarker is one or more of a tumor mutational burden (TMB) score, a homologous recombination deficiency (HRD) score, or a microsatellite instability (MSI) status.

Embodiment 25. The method of embodiment 24, wherein the TMB score is at least about 4 to 100 mutations/Mb, about 4 to 30 mutations/Mb, 8 to 100 mutations/Mb, 8 to 30 mutations/Mb, 10 to 20 mutations/Mb, less than 4 mutations/Mb, or less than 8 mutations/Mb.

Embodiment 26. The method of embodiment 25, wherein the TMB is at least about 5 mutations/Mb.

Embodiment 27. The method of embodiment 25 or embodiment 26, wherein the TMB score is at least about 10 mutations/Mb.

Embodiment 28. The method of any one of embodiments 25-27, wherein the TMB score is at least about 12 mutations/Mb.

Embodiment 29. The method of any one of embodiments 25-28, wherein the TMB score is at least about 16 mutations/Mb.

Embodiment 30. The method of any one of embodiments 25-29, wherein the TMB score is at least about 20 mutations/Mb.

Embodiment 31. The method of any one of embodiments 25-30, wherein the TMB score is at least about 30 mutations/Mb.

Embodiment 32. The method of any one of embodiments 25-31, wherein the TMB score is determined based on between about 100 kb to about 10 Mb.

Embodiment 33. The method of any one of embodiments 25-32, wherein the TMB score is determined based on between about 0.8 Mb to about 1.1 Mb.

Embodiment 34. The method of any one of embodiments 24-33, wherein the TMB score is a blood TMB (bTMB) score.

Embodiment 35. The method of embodiment 24, wherein the MSI status is a MSI high or MSI low status.

Embodiment 36. The method of embodiment 24, wherein the MSI status is an MSI stable status.

Embodiment 37. The method of embodiment 24, wherein the HRD score is a HRD-positive score, or a HRD-negative score.

Embodiment 38. The method of embodiment 23, wherein the biomarker comprises one or more alterations in one or more of ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, C11orf30, C17orf39, CALR, CARD11, CASP8, CBFB, CBL, CCND1, CCND2, CCND3, CCNE1, CD22, CD70, CD74, CD79A, CD79B, CD274, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A, KMT2D, KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NSD3, NT5C2, NTRK1 NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK. TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WTI, XPO1, XRCC2, ZNF217, and ZNF703, or any combination thereof.

Embodiment 39. The method of embodiment 23, wherein the biomarker comprises one or more alteration in PIK3CA.

Embodiment 40. The method of embodiment 38 or embodiment 39, wherein of the one or more alterations comprise a base substitution, an insertion/deletion (indel), a copy number alteration, or a genomic rearrangement.

Embodiment 41. The method of any one of embodiments 1-40, wherein the tumor shed value is determined by composite tumor fraction (cTF) or by a tumor fraction estimator (TFE) process.

Embodiment 42. The method of embodiment 41, wherein the tumor shed value is determined by cTF using a method comprising:
(a) receiving a plurality of values, each value indicative of an allele fraction at a corresponding locus within a subgenomic interval in the sample;
(b) determining a certainty metric value indicative of a dispersion of the plurality of values;
(c) determining a first estimate of the tumor fraction of the sample, the first estimate based on the certainty metric value for the sample and a predetermined relationship between one or more stored certainty metric values and one or more stored tumor fraction values;
(d) determining whether a value associated with the first estimate is greater than a first threshold, wherein if the value associated with the first estimate is greater than the first threshold, the first estimate determined as the tumor fraction of the sample; and
(e) if the value associated with the first estimate is less than or equal to the first threshold, determining a second estimate of the tumor fraction of the sample based on an allele frequency determination, wherein the second estimate is determined as the tumor fraction of the sample.

Embodiment 43. The method of embodiment 42, wherein the tumor fraction is a value indicative of a ratio of circulating tumor DNA (ctDNA) to total cell-free DNA (cfDNA) in the sample.

Embodiment 44. The method of embodiment 42 or embodiment 43, wherein the first threshold is indicative of a minimum detectable quantity for the tumor fraction of the sample.

Embodiment 45. The method of any one of embodiments 42-44, wherein determining whether the value associated with the first estimate of the tumor fraction of the sample is greater than the first threshold comprises determining whether the first estimate is greater than a defined tumor fraction threshold.

Embodiment 46. The method of any one of embodiments 42-45, wherein determining whether the value associated with the first estimate of the tumor fraction of the sample is greater than a first threshold comprises determining whether a statistical lower bound associated with the first estimate is greater than 0.

Embodiment 47. The method of any one of embodiments 42-46, wherein determining the second estimate of the tumor fraction of the sample based on the allele frequency determination comprises:
(a) determining whether a quality metric for the plurality of values is greater than a second threshold;
(b) based on a determination that the quality metric for the plurality of values is greater than the second threshold, determining the second estimate for the tumor fraction of the sample based on a first determination of somatic allele frequency, and
(c) based on a determination that the quality metric for the plurality of values is less than or equal to the second threshold, determining the second estimate for the tumor fraction of the sample based on a second determination of somatic allele frequency.

Embodiment 48. The method of embodiment 47, wherein the quality metric for the plurality of values is indicative of an average sequence coverage for the sample, an allele coverage at each loci corresponding to the plurality of values, a degree of nucleic acid contamination in the sample, a number of single nucleotide polymorphism (SNP) loci within the loci corresponding to the plurality of values, or any combination thereof.

Embodiment 49. The method of embodiment 48, wherein the quality metric for the plurality of values is indicative of a minimum average sequence coverage for the sample, a minimum allele coverage at each of the loci corresponding to the plurality of values, a maximum degree of nucleic acid contamination in the sample, a minimum number of single nucleotide polymorphism (SNP) loci within the loci corresponding to the plurality of values, or any combination thereof.

Embodiment 50. The method of any one of embodiments 47-49, wherein the second threshold comprises a specified lower limit of the quality metric.

Embodiment 51. The method of any one of embodiments 47-50, wherein the first determination of somatic allele frequency comprises a determination of variant allele frequencies associated with the plurality of values after excluding variant alleles that are present at an allele frequency greater than an upper bound for the first estimate of the tumor fraction of the sample, and the second estimate of the tumor fraction of the sample is set equal to a maximum variant allele frequency detected.

Embodiment 52. The method of any one of embodiments 47-51, wherein the second determination of somatic allele frequency comprises a determination of variant allele frequencies for all variant alleles associated with the plurality of values, and the second estimate of the tumor fraction of the sample is set equal to a maximum variant allele frequency detected.

Embodiment 53. The method of any one of embodiments 47-52, wherein the first determination of somatic allele frequency and the second determination of somatic allele frequency further comprise removing variant allele frequencies from the determination that correspond to germline variants, clonal hematopoiesis of indeterminate potential (CHIP) variants, and sequencing artifact variants, prior to determining the second estimate of the tumor fraction of the sample.

Embodiment 54. The method of embodiment 53, wherein the first determination of somatic allele frequency and the second determination of somatic allele frequency further comprise using a variant allele frequency for a rearrangement as the second estimate of the tumor fraction of the sample if rearrangements are detected in the sample.

Embodiment 55. The method of any one of embodiments 47-54, wherein the first determination of somatic allele frequency and the second determination of somatic allele frequency further comprise removing variant allele frequencies that correspond to variants of unknown significance prior to determining the second estimate of the tumor fraction of the sample.

Embodiment 56. The method of any one of embodiments 47-55, wherein each value within the plurality of values is an allele fraction.

Embodiment 57. The method of any one of embodiments 47-56, wherein each value within the plurality of values comprises a ratio of the difference in abundance between a maternal allele and a paternal allele relative to abundance of the maternal allele or the paternal allele at the corresponding locus.

Embodiment 58. The method of any one of embodiments 47-57, wherein the certainty metric value for the sample is indicative of a deviation of each of the plurality of values from a corresponding expected value.

Embodiment 59. The method of embodiment 58, wherein the corresponding expected value is a locus-specific expected value.

Embodiment 60. The method of embodiment 58 or embodiment 59, wherein the certainty metric for the sample is a root mean squared deviation of the plurality of values from their corresponding expected values.

Embodiment 61. The method of any one of embodiments 58-60, wherein the corresponding expected value is an expected allele frequency for a non-tumorous sample.

Embodiment 62. The method of any one of embodiments 58-61, wherein each value within the plurality of values is an allele fraction, and the expected value is about 0.5.

Embodiment 63. The method of any one of embodiments 58-62, wherein each value within the plurality of values is a ratio of the difference in abundance between a maternal allele and a paternal allele, relative to an abundance of the maternal allele or the paternal allele at the corresponding locus, and the expected value comprises the expected ratio of the difference in abundance between a maternal allele and a paternal allele, relative to an abundance of the maternal allele or the paternal allele, wherein the expected value is the expected ratio for a non-tumorous sample.

Embodiment 64. The method of embodiment 63, wherein the corresponding expected value is about 0.

Embodiment 65. The method of any one of embodiments 47-64, wherein the plurality of values comprises a plurality of allele coverages.

Embodiment 66. The method of any one of embodiments 47-65, further comprising determining a probability distribution function for the plurality of values; wherein the certainty metric value for the sample is determined using the probability distribution function.

Embodiment 67. The method of embodiment 66, wherein the certainty metric value for the sample is an entropy of the probability distribution function.

Embodiment 68. The method of any one of embodiments 47-67, wherein the corresponding loci comprise one or more loci having a different maternal allele and paternal allele.

Embodiment 69. The method of any one of embodiments 47-67, wherein the corresponding loci consist of loci having a different maternal allele and paternal allele.

Embodiment 70. The method of any one of embodiments 47-67, wherein the corresponding loci comprise one or more loci having the same maternal allele and paternal allele.

Embodiment 71. The method of embodiment 41, wherein the tumor shed value is determined by a TFE process using a method comprising:
(a) receiving a plurality of values, each value indicative of an allele fraction at a corresponding locus within a subgenomic interval in the sample;
(b) determining a certainty metric value indicative of a dispersion of the plurality of values; and
(c) determining an estimate of the tumor fraction of the sample based on the certainty metric value for the sample and a predetermined relationship between one or more stored certainty metric values and one or more stored tumor fraction values, wherein the estimate is determined as the tumor fraction of the sample.

Embodiment 72. The method of embodiment 71, wherein the tumor fraction is a value indicative of a ratio of ctDNA to total cfDNA in the sample.

Embodiment 73. The method of embodiment 71 or embodiment 72, wherein each value within the plurality of values is an allele fraction.

Embodiment 74. The method of any one of embodiments 71-73, wherein each value within the plurality of values comprises a ratio of the difference in abundance between a maternal allele and a paternal allele relative to abundance of the maternal allele or the paternal allele at the corresponding locus.

Embodiment 75. The method of any one of embodiments 71-74, wherein the certainty metric value for the sample is indicative of a deviation of each of the plurality of values from a corresponding expected value.

Embodiment 76. The method of any one of embodiments 71-75, wherein the plurality of values comprises a plurality of allele coverages.

Embodiment 77. The method of any one of embodiments 71-76, further comprising determining a probability distribution function for the plurality of values; wherein the certainty metric value for the sample is determined using the probability distribution function.

Embodiment 78. The method of embodiment 77, wherein the certainty metric value for the sample is an entropy of the probability distribution function.

Embodiment 79. The method of any one of embodiments 71-78, wherein the corresponding loci comprise one or more loci having a different maternal allele and paternal allele.

Embodiment 80. The method of any one of embodiments 71-78, wherein the corresponding loci consist of loci having a different maternal allele and paternal allele.

Embodiment 81. The method of any one of embodiments 71-78, wherein the corresponding loci comprise one or more loci having the same maternal allele and paternal allele.

Embodiment 82. The method of any one of embodiments 1-81, wherein the reference tumor shed value is between 0.5% to 10.0%.

Embodiment 83. The method of any one of embodiments 1-81, wherein the reference tumor shed value is 0.5%.

Embodiment 84. The method of any one of embodiments 1-81, wherein the reference tumor shed value is 1.0%.

Embodiment 85. The method of any one of embodiments 1-81, wherein the reference tumor shed value is 2.0%.

Embodiment 86. The method of any one of embodiments 1-13, 21-22 and 41-85, wherein the IO therapy comprises a single IO agent or multiple IO agents.

Embodiment 87. The method of any one of embodiments 1-13, 21-22 and 41-86, wherein the IO therapy comprises an immune checkpoint inhibitor.

Embodiment 88. The method of embodiment 87, wherein the immune checkpoint inhibitor comprises a small molecule inhibitor, an antibody, a nucleic acid, an antibody-drug conjugate, a recombinant protein, a fusion protein, a natural compound, a peptide, a PROteolysis-TArgeting Chimera (PROTAC), a cellular therapy, a treatment for cancer being tested in a clinical trial, an immunotherapy, or any combination thereof.

Embodiment 89. The method of embodiment 87 or embodiment 88, wherein the immune checkpoint inhibitor is a PD-1 inhibitor.

Embodiment 90. The method of embodiment 89, wherein the immune checkpoint inhibitor comprises one or more of nivolumab, pembrolizumab, cemiplimab, or dostarlimab.

Embodiment 91. The method of embodiment 87 or embodiment 88, wherein the immune checkpoint inhibitor is a PD-L1-inhibitor.

Embodiment 92. The method of embodiment 91, wherein the immune checkpoint inhibitor comprises one or more of atezolizumab, avelumab, or durvalumab.

Embodiment 93. The method of embodiment 87 or embodiment 88, wherein the immune checkpoint inhibitor is a CTLA-4 inhibitor.

Embodiment 94. The method of embodiment 93, wherein the CTLA-4 inhibitor comprises ipilimumab.

Embodiment 95. The method of embodiment 88, wherein the nucleic acid comprises a double-stranded RNA (dsRNA), a small interfering RNA (siRNA), or a small hairpin RNA (shRNA).

Embodiment 96. The method of 88, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 97. The method of any one of embodiments 1-6, 13, 22 and 41-96, wherein the chemotherapy comprises a single chemotherapeutic agent or multiple therapeutic agents.

Embodiment 98. The method of any one of embodiments 1-6, 13, 22 and 41-97, wherein the chemotherapy comprises one or more of an alkylating agent, an alkyl sulfonates aziridine, an ethylenimine, a methylamelamine, an acetogenin, a camptothecin, a bryostatin, a callystatin, CC-1065, a cryptophycin, aa dolastatin, a duocarmycin, a eleutherobin, a pancratistatin, a sarcodictyin, a spongistatin, a nitrogen mustard, a nitrosureas, an antibiotic, a dynemicin, a bisphosphonate, an esperamicina a neocarzinostatin chromophore or a related chromoprotein enediyne antiobiotic chromophore, an anti-metabolite, a folic acid analogue, a purine analog, a pyrimidine analog, an androgens, an anti-adrenal, a folic acid replenisher, aldophosphamide glycoside, aminolevulinic acid, eniluracil, amsacrine, bestrabucil, bisantrene, edatraxate, defofamine, demecolcine, diaziquone, elformithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid, 2-ethylhydrazide, procarbazine, a PSK polysaccharide complex, razoxane, rhizoxin, sizofiran, spirogermanium, tenuazonic acid, triaziquone, 2,2′,2″-trichlorotriethylamine, a trichothecene, urethan, vindesine, dacarbazine, mannomustine, mitobronitol, mitolactol, pipobroman, gacytosine, arabinoside (“Ara-C”), cyclophosphamide, a taxoid, 6-thioguanine, mercaptopurine, a platinum coordination complex, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan, topoisomerase inhibitor RFS 2000, difluorometlhylomithine (DMFO), a retinoid, capecitabine, carboplatin, procarbazine, plicomycin, gemcitabine, navelbine, a famesyl-protein tansferase inhibitor, transplatinum, or any combination thereof.

Embodiment 99. The method of any one of embodiments 5-6, 1.-121, 18-19 and 41-98, wherein the survival is progression-free survival (PFS).

Embodiment 100. The method of any one of embodiments 5-6, 11-12, 18-19 and 41-98, wherein the survival is overall survival (OS).

Embodiment 101. The method of any one of embodiments 1-6, 13, 21-22 and 41-100, further comprising treating the individual with the IO therapy in combination with chemotherapy.

Embodiment 102. The method of embodiment 101, wherein the IO therapy and the chemotherapy are administered concurrently or sequentially.

Embodiment 103. The method of any one of embodiments 7-13, 21 and 41-100, further comprising treating the individual with the IO therapy.

Embodiment 104. The method of any one of embodiments 24-34 and 41-85, further comprising treating the individual with a TMB-targeted therapy.

Embodiment 105. The method of embodiment 104, wherein the TMB-targeted therapy comprises an immune checkpoint inhibitor.

Embodiment 106. The method of embodiment 105, wherein the immune checkpoint inhibitor is an anti-PD1 therapy or an anti-PD-L1 therapy.

Embodiment 107. The method of embodiment 106, wherein the anti-PD-1 therapy comprises one or more of nivolumab, pembrolizumab, cemiplimab, or dostarlimab.

Embodiment 108. The method of embodiment 106, wherein the anti-PD-L1 therapy comprises one or more of atezolizumab, avelumab, or durvalumab.

Embodiment 109. The method of embodiment 24, 35 and 41-85, further comprising treating the individual with a MSI high status an MSI-high-targeted therapy.

Embodiment 110. The method of embodiment 109, wherein the MSI-high-targeted therapy comprises an immune checkpoint inhibitor.

Embodiment 111. The method of embodiment 110, wherein the immune checkpoint inhibitor is an anti-PD1 therapy, an anti-PD-L1 therapy, or an anti-CTLA-4 therapy.

Embodiment 112. The method of embodiment 111, wherein the anti-PD-1 therapy comprises one or more of nivolumab, pembrolizumab, cemiplimab, or dostarlimab.

Embodiment 113. The method of embodiment 111, wherein the anti-PD-L1 therapy comprises one or more of atezolizumab, avelumab, or durvalumab.

Embodiment 114. The method of embodiment 111, wherein the anti-CTLA-4 therapy comprises ipilimumab.

Embodiment 115. The method of embodiment 24, 37 and 41-85, further comprising treating the individual having a HRD-positive score with an HRD-positive targeted therapy.

Embodiment 116. The method of embodiment 115, wherein the HRD-positive targeted therapy is selected from the group consisting of a platinum-based drug and a PARP inhibitor, or any combination thereof.

Embodiment 117. The method of embodiment 115, wherein the PARP inhibitor is olaparib, niraparib, or rucaparib.

Embodiment 118. The method of any one of embodiments 1-117, further comprising treating the individual with an additional anti-cancer therapy.

Embodiment 119. The method of embodiment 1118, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

Embodiment 120. The method of any one of embodiments 1-119, wherein the liquid biopsy sample comprises blood, plasma, serum, cerebrospinal fluid, sputum, stool, urine, or saliva.

Embodiment 121. The method of embodiment 120, wherein the liquid biopsy is blood, plasma, or serum.

Embodiment 122. The method of any one of embodiments 1-121, wherein the liquid biopsy sample comprises mRNA, DNA, circulating tumor DNA (ctDNA), cell-free DNA, or cell-free RNA from the cancer.

Embodiment 123. The method of any one of embodiments 1-122, wherein the tumor shed value is determined by sequencing.

Embodiment 124. The method of embodiment 123, wherein the sequencing comprises use of a massively parallel sequencing (MPS) technique, whole genome sequencing (WGS), whole exome sequencing, targeted sequencing, direct sequencing, next-generation sequencing (NGS), or a Sanger sequencing technique.

Embodiment 125. The method of embodiment 123 or embodiment 124, wherein the sequencing comprises:
(a) providing a plurality of nucleic acid molecules obtained from the sample, wherein the plurality of nucleic acid molecules comprises a mixture of tumor nucleic acid molecules and non-tumor nucleic acid molecules;
(b) optionally, ligating one or more adapters onto one or more nucleic acid molecules from the plurality of nucleic acid molecules;
(c) amplifying nucleic acid molecules from the plurality of nucleic acid molecules;
(d) capturing nucleic acid molecules from the amplified nucleic acid molecules, wherein the captured nucleic acid molecules are captured from the amplified nucleic acid molecules by hybridization to one or more bait molecules;
(e) sequencing, by a sequencer, the captured nucleic acid molecules to obtain a plurality of sequence reads corresponding to one or more genomic loci within a subgenomic interval in the sample.

Embodiment 126. The method of embodiment 125, wherein the adapters comprise one or more of amplification primer sequences, flow cell adapter hybridization sequences, unique molecular identifier sequences, substrate adapter sequences, or sample index sequences.

Embodiment 127. The method of embodiment 125 or embodiment 126, wherein amplifying nucleic acid molecules comprises performing a polymerase chain reaction (PCR) technique, a non-PCR amplification technique, or an isothermal amplification technique.

Embodiment 128. The method of any one of embodiments 125-127, wherein the one or more bait molecules comprise one or more nucleic acid molecules, each comprising a region that is complementary to a region of a captured nucleic acid molecule.

Embodiment 129. The method of embodiment 128, wherein the one or more bait molecules each comprise a capture moiety.

Embodiment 130. The method of embodiment 129, wherein the capture moiety is biotin.

Embodiment 131. The method of any one of embodiments 1-30, wherein the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Embodiment 132. The method of embodiment 131, wherein the cancer is a NSCLC, colorectal cancer, cholangiocarcinoma, breast cancer, stomach cancer, melanoma, pancreatic cancer, prostate cancer, ovarian cancer, esophageal cancer, or a cancer of unknown primary.

Embodiment 133. The method of any of embodiments 1-132, wherein the individual is a human.

Embodiment 134. The method of any one of embodiments 1-133, wherein the individual has previously been treated with an anti-cancer therapy.

Embodiment 135. The method of embodiment 134, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

### EXAMPLES

### Example 1: Comparison of Tumor Diagnosis of Matched Tissue and Liquid Biopsy Samples Stratified by Tumor Shed

This Example describes the comparison of tumor status diagnosis in liquid biopsy samples stratified by two tumor shed determination methods.

### Materials and Methods

### Liquid Biopsy Assay

The liquid biopsy assay was performed in a Clinical Laboratory Improvement Amendments (CLIA)-certified and College of American Pathologists (CAP)-accredited laboratory. The liquid biopsy assay analyzes cell-free DNA (cfDNA) isolated from plasma using a next generation sequencing platform and a targeted hybrid capture methodology that detects base substitutions, insertions and deletions 311 commonly altered oncogenes, gene rearrangements in four genes, and copy number alterations in three genes.

### Determination of Tumor Shed

The composite tumor fraction (cTF) measures the circulating tumor DNA (ctDNA) fraction of the total circulating cfDNA, across a broad range of tumor content values. The method leverages two complementary metrics which are a proprietary tumor fraction estimator (TFE) and the maximum allele frequency (MAF) method.

The TFE is based on a measure of tumor aneuploidy that incorporates observed deviations in variant coverage across the genome for a given sample. Calculated values for this metric are calibrated against a training set based on samples with well-defined tumor fractions to generate an estimate of the tumor fraction in a sample. The MAF estimates the fraction of ctDNA versus that of all sources of cfDNA in plasma. The MAF is determined by determining the allele fraction for all known somatic, likely somatic, and variant of unknown significance (VUS) substitution alterations detected at >2000× median unique coverage by non-PCR duplicate read pairs, excluding certain common and rare germline variants, as well as a select list of variants associated with clonal hematopoiesis. cTF defaults to the TFE's value when available. When lack of tumor aneuploidy limits the TFE's ability to return an informative estimate of TF, MAF is used to generate a cTF value. In this case, only variants with allele fraction at or below TFE's limit of detection are used as input for MAF. When the TFE's estimate is unavailable or impacted by failed quality control measures, the cTF value is generated from MAF as previously described. A second version of cTF (cTF v2) was also developed and used. cTF v2 uses known and likely short variants, as well as known fusion rearrangements, for MAF determination. Moreover, cTF v2 excludes additional genes (ATM, CHEK2, and GNAS), low confidence MSH3 non-frameshifts mutations, and VUS from the analysis.

The cTF measurement defaults to the TFE's value when available. When lack of tumor aneuploidy limits the TFE's ability to return an informative estimate of ctDNA fraction, MAF was used to generate a ctDNA fraction value. In this case, only variants with allele fraction at or below TFE's limit of detection are used as input for MAF. When the TFE's estimate was unavailable or impacted by failed quality control measures, the cTF or cTF v2 was generated from MAF as described above.

### Patient Stratification

Patients with non-small cell lung cancer (NSCLC) who had both a tissue-based tumor test and a subsequent liquid biopsy-based comprehensive genomic testing were included in the study. Overall, 247/701 (35%) and of samples were determined to be tumor positive and 454/701 (65%) as tumor unknown by a liquid biopsy-based test, while 379/701 (54%) and 322/701 (46%) were found to be tumor positive and tumor unknown, respectively, by a tissue-based test. The specimens were then stratified by tumor shed as determined by MAF, composite tumor fraction (cTF) or cTF v2.

Patients with a MAF≥1% were considered “tumor present,” while all other MAF values were considered “tumor unknown.” For tumor shed determination by composite tumor fraction (cTF) and cTF v2, patients with cTF≥1% were considered “tumor present”, and all other cTF values were considered “tumor unknown.”

### Statistical Analysis

Data from the NSCLC specimens was sampled 1,000 times in a simulated model. The positive percent agreement (PPA), negative percent agreement (NPA), positive predictive value (PPV), and negative predictive value (NPV) were calculated across all four measures for both the tumor-present and tumor-unknown cohorts. Table 1 summarizes the comparison of tumor driver status diagnosis between tissue-based and liquid-biopsy based test.

TABLE 1

| | Tumor driver | Tumor driver |
|---|---|---|
| | positive status by | negative status by |
| | tissue-based test | tissue-based test |
| Tumor driver positive by | True Positive | False Positive |
| liquid biopsy-based test | | |
| Tumor driver negative by | False Negative | True Negative |
| liquid biopsy-based test | | |

### Results

To establish whether a tumor shed cut-off would lead to improved PPA and NPV in liquid biopsies, NSCLC liquid samples were classified as tumor positive or negative based on a tumor shed cut-off of 1%. Tumor shed was then determined based on MAF or cTF. At a cutoff of 1% MAF, 610 (87%) samples were classified as tumor positive, and 91 (13%) samples were classified as not detected or unknown. For cTF, using a cutoff of 1%, 488 (70%) samples were classified as tumor positive and 213 (30%) samples were classified as not detected or unknown, while for cTF v2, 356 (51%) samples were classified as tumor positive, and 345 (49%) samples classified as not detected or unknown.

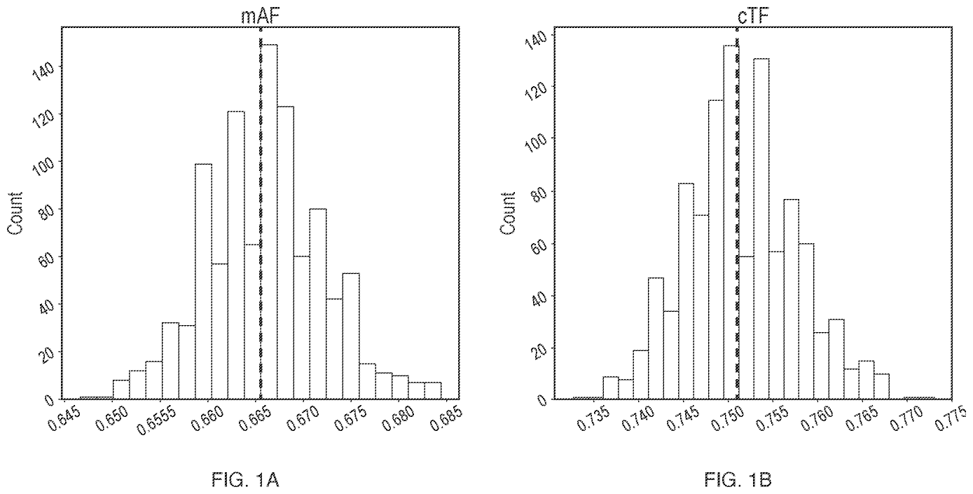

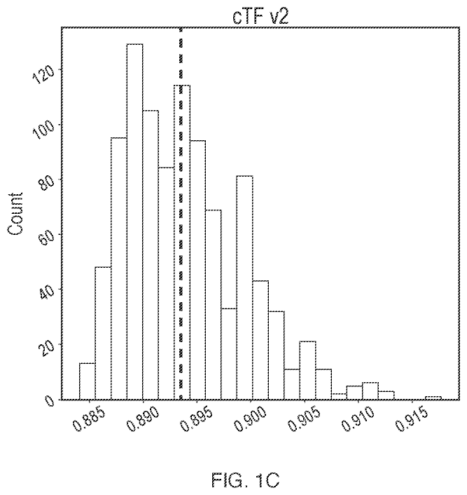

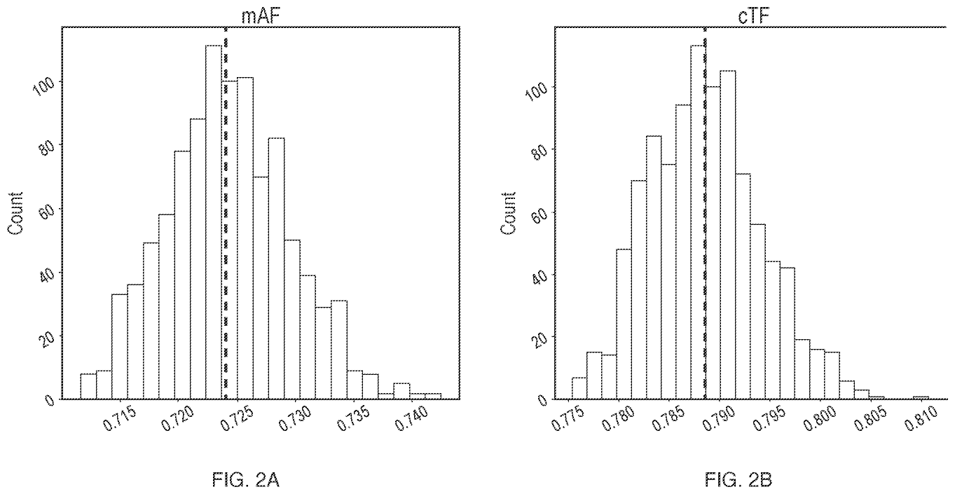

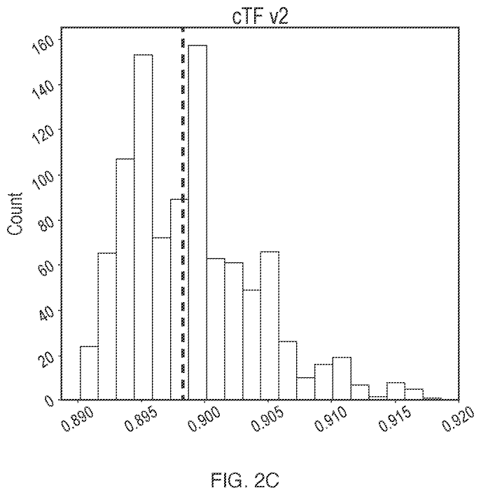

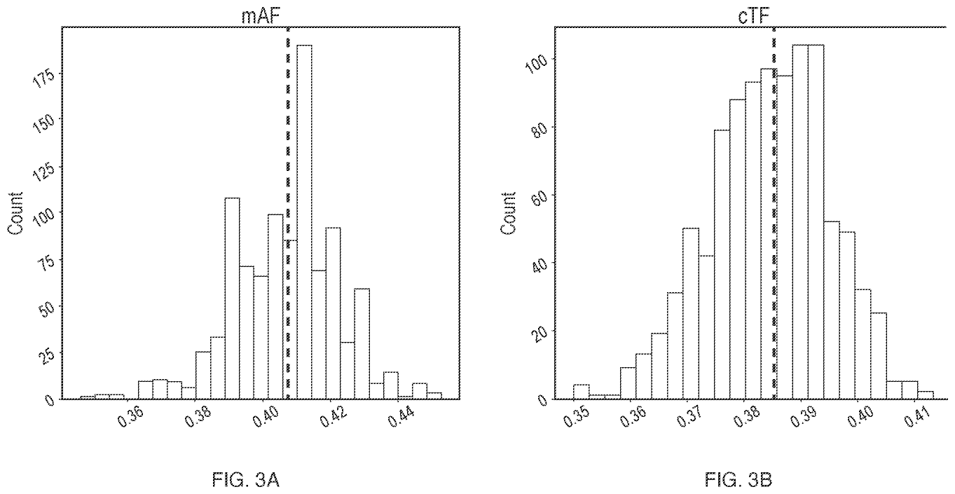

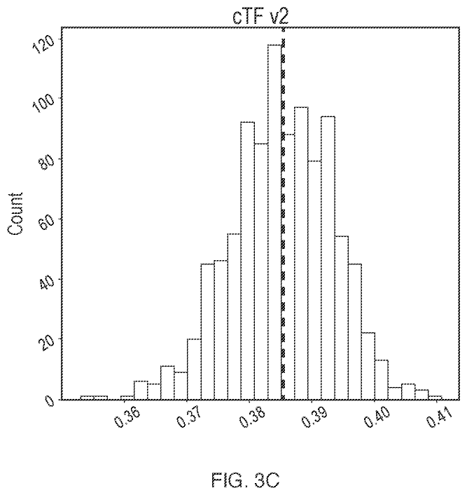

For each measure of tumor shed, the data was split into training and testing data sets, and a logistic regression model was trained. The PPA and NPV values were calculated for each simulation. The PPA for tumor-present specimens was higher for cTF-stratified specimens than for MAF-stratified specimens. The tumor-present PPA was 67% for MAF-, 75% for cTF-, and 90% for cTFv2-stratified specimens (FIGS. 1A-1B). For tumor-unknown specimens, the PPA was comparable, with a PPA of 41%, 39%, and 38% for MAF-, cTF-, and cTF v2-stratified specimens, respectively (FIGS. 3A-3B). NPV was also found to be increased in cTF-stratified specimens, with 72%, 79%, and 90% for MAF-, cTF-, and cTF v2-stratified specimens, respectively (FIGS. 2A-2B).

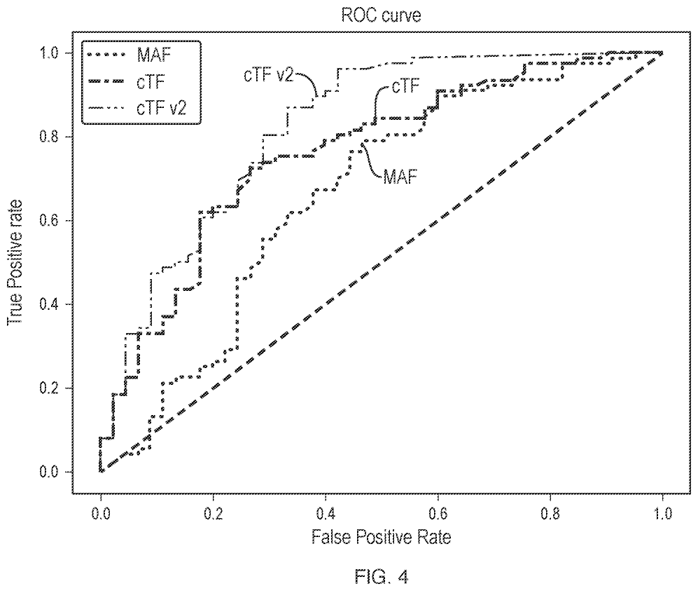

Receiver operator characteristic (ROC) curves were also generated for MAF, cTF and cTF v2 measurements of tumor shed in liquid biopsy samples. The area under the curve (AUC) was higher for cTF and cTF v2 stratification than for MAF stratification (FIG. 4). The higher AUC in the models using cTF and cTF v2 are indicative of higher accuracy for defining tumor positive or tumor unknown status when using these models.

### Conclusions

cTF and cTF v2 showed increased PPA and NPV for defining tumor status in liquid biopsy specimens when using a cut-off of 1%. Determination of tumor status of liquid biopsy specimens stratified by tumor shed based on cTF and cTF v2 was superior to the commonly used MAF measurement.

### Example 2: Predicting Response to Immune-Oncology Therapy Based on Tumor Shed Status

This Example describes the prediction of response to immune-oncology (IO) monotherapy based on tumor shed measurements.

### Materials and Methods

### Tumor Shed Stratification

NSCLC patients treated with IO monotherapy, or a combination of IO therapy and chemotherapy, and with liquid biopsy no more than 60 days prior to start of therapy, were stratified by cTF or MAF tumor shed measurements, using a cut-off value of 1%. Overall survival, time to next therapy and progression-free survival were calculated for the patient cohorts, with overall survival adjusted for delayed entry into the study.

### Results

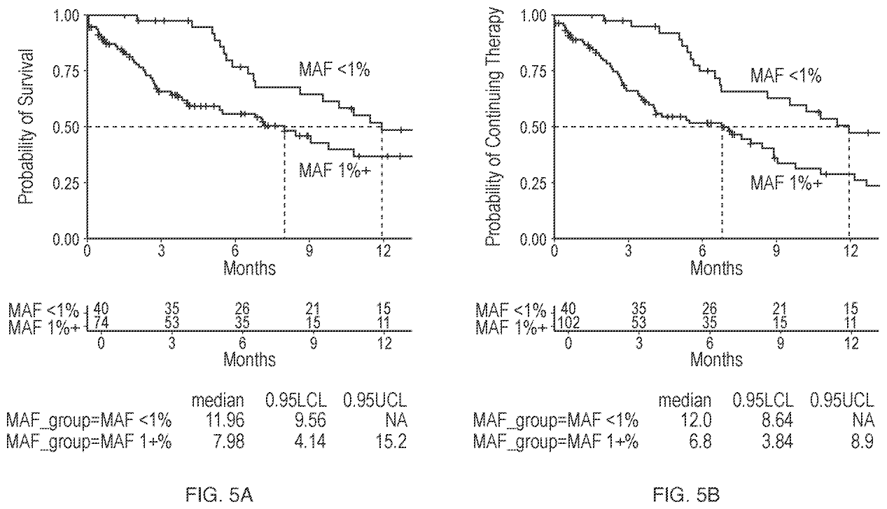

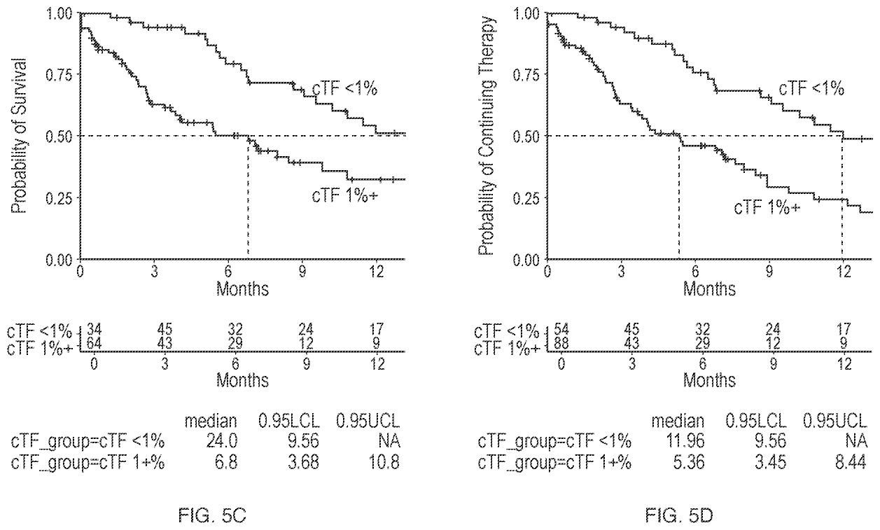

Overall survival and time to next therapy were calculated in NSCLC patients treated with IO monotherapy stratified by tumor shed (FIGS. 5A-5D). 14 patients were classified as ≤1% for cTF that were then considered >1% by MAF. Patients with ≤1% cTF showed improved overall survival and time to next therapy on an IO monotherapy regimen (FIGS. 5C-5D). Additionally, though patients with ≤1% MAF also showed improved overall survival (FIGS. 5A-5B), the difference in median overall survival between patients with ≤1% or >1% tumor shed was greater for cTF than for MAF, suggesting improved stratification when using cTF.

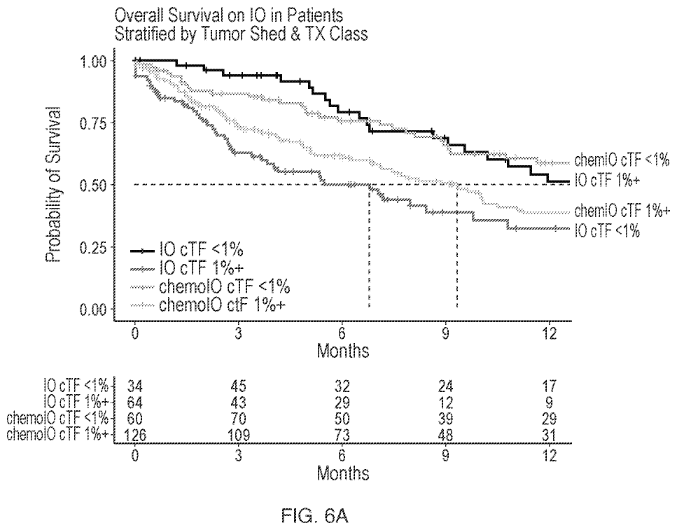

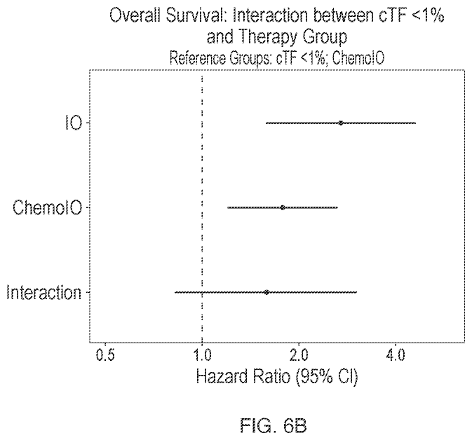

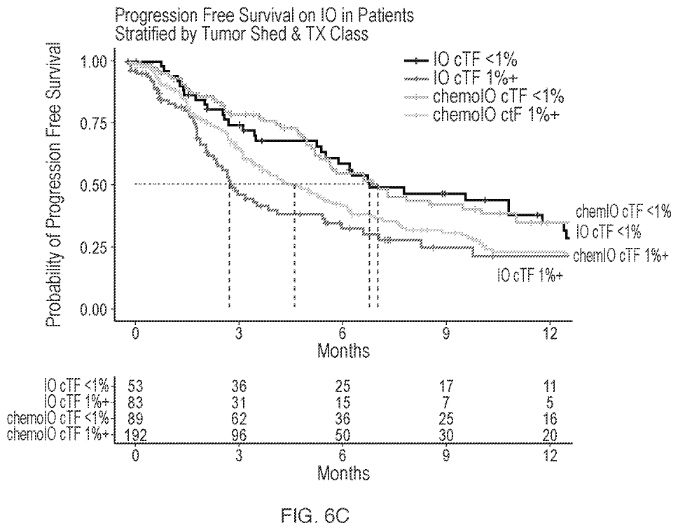

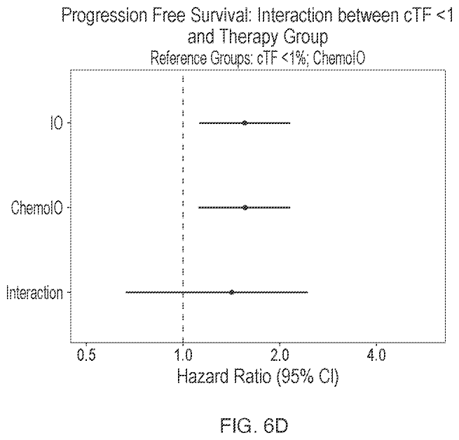

Overall survival and real-world progression free survival were calculated in NSCLC patients treated with IO monotherapy or chemotherapy in combination with IO (FIGS. 6A-6D). Patients with ≤1% cTF showed improved overall survival and progression-free survival in both the IO, and IO and chemotherapy combination cohorts (FIGS. 6A and 6C), which is suggestive of a prognostic use for this measurement. Moreover, the therapy and cTF interaction term from a Cox Proportional Hazard model suggests that <1% cTF predicts improved outcomes for patients with received IOP monotherapy than for those that received a combination of chemotherapy and IO (FIGS. 6B and 6D). Thus, cTF could be used as a predictive biomarker for identifying patients who would benefit more from an IO monotherapy than the combination of IO therapy with chemotherapy based on a cut-off of 1% cTF.

### Conclusions

cTF shows prognostic value for patients treated with IO monotherapy or a combination of IO therapy with chemotherapy. Moreover, cTF may be used as a predictive biomarker for identifying patients that would benefit more from an IO monotherapy as compared to a combination of IO therapy with chemotherapy.

### Example 3: Effect of Tumor Fraction on the Detection of Biomarker Variants in Liquid Biopsy Samples

This example describes the investigation of the effect of tumor fraction on detection of biomarker variants in liquid biopsy samples from cancer patients.

### Materials and Methods

Paired tissue and liquid biopsy samples from 206 patients with breast cancer with available next generation sequencing results were collected for a median time of 12 months (IQR: 1.2, 27). Positive percent agreements (PPA) were calculated to determine concordance of tumor status between the paired samples at both patient and variant level. The PPA at patient-level was calculated with tissue results as standard.

The tumor samples were stratified by tumor shed using cTF minimum cut-offs of 0%, 0.5%, 1%, 2%, 5%, and 10% cTF. The patient level PPA for detection of PIK3CA variants as well as the fraction of the total cohort stratified by the cTF cut-off were calculated for each cTF cut-off value.

### Results

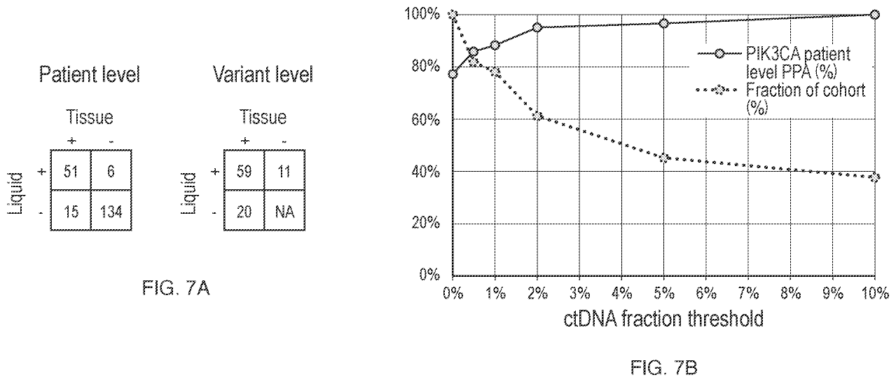

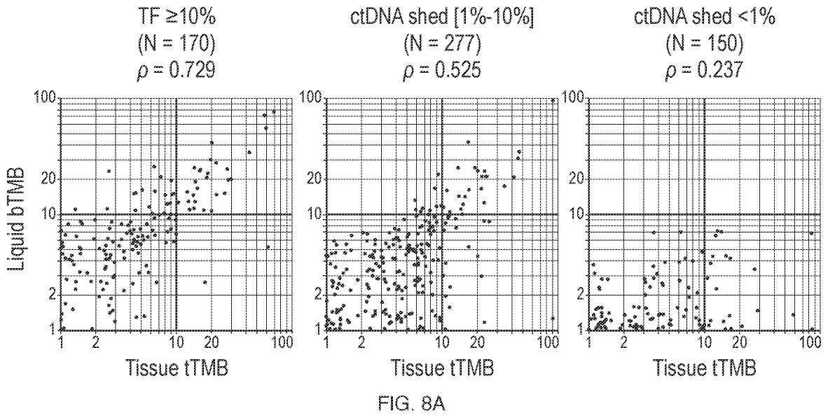

FIG. 7A shows the concordance for PIK3CA variant detection in liquid and tissue biopsy. The PPA for the paired was 77% (51/66) and 75% (59/79) at patient- and variant-level, respectively.

The effect of the tumor shed on variant detection was further evaluated. FIG. 7B shows the effect of the tumor shed on PIK3CA patient level PPA (solid line) and fraction of cohort (dotted line). Table 2 summarizes the PIK3CA patient level PPA and fraction of the total cohort represented at different cTF cut-offs. A cTF cut-off of 0.5% showed good correlation for PIK3CA variant detection in tissue and liquid biopsy samples (86%), and represented 82% of the total cohort. Optimal cTF thresholds may differ for other types of variants and complex genomic biomarkers.

TABLE 2
Effect of ctDNA fraction on PPA

| cTF threshold | PIK3CA patient level PPA (%) | Fraction of cohort (%) |
|---|---|---|
| 0% | 77% | 100% |
| 0.5% | 86% | 82% |
| 1% | 88% | 78% |
| 2% | 95% | 62% |
| 5% | 97% | 46% |
| 10% | 100% | 37% |

### Conclusions

The detection of traditional biomarkers, such as PIK3CA variants, in liquid biopsy samples is better correlated with tumor biopsy results as the cTF increases.

### Example 4: Association of Tumor Shed with Complex Genomic Biomarkers in Liquid Biopsy Samples

This example describes the investigation of the effect of tumor fraction on the detection of complex genomic biomarkers in liquid biopsy samples from cancer patients.

### Materials and Methods

### Effect of Tumor Shed on Tumor Mutation Burden

A pan-cancer cohort of 597 patient-matched liquid biopsy (blood) and tumor biopsy samples were collected >90 days apart, where both tissue and liquid biopsies passed quality control.

Tumor shed was calculated by cTF, and the paired samples were stratified as cTF≥10%, cTF≥1% but lower than 10%, and cTF<1% (including samples with cTF=0%). The tumor mutation burden (TMB) was calculated for both the liquid and tissue biopsy samples, using a threshold of 10 mutations/Mb threshold for tissue TMB (tTMB) determination, and 10 and 14 mutations/Mb thresholds for blood TMB (bTMB) determination. The correlation between the tTMB and bTMB was quantified by Spearman's correlation coefficient (ρ) and Lin's Concordance Correlation Coefficient (CCC). For each cTF group, the PPA, negative percent agreement (NPA), and overall percent agreement (OPA) were calculated for both bTMB>10 and bTMB>14 thresholds, with tTMB≥10 used as standard for both.

### Distribution of Complex Biomarkers at Different Tumor Fractions

A cohort of 16,381 patients with available liquid biopsy results for cTF, bTMB, and microsatellite instability (MSI) status were analyzed. The cTF distribution of the entire cohort as well as each subset cohort (bTMB<10 mut/Mb, bTMB>10 mut/Mb, MSI-H) is shown.

### Results

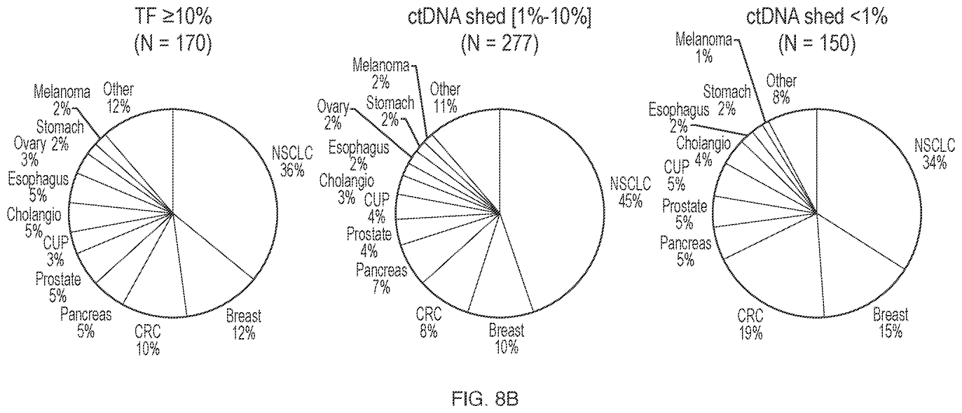

Samples with cTF≥10% showed the strongest correlation between tTMB and bTMB results (ρ=0.729), followed by the cTF=≥1% but lower than 10% group (ρ=0.0.525), whereas the <1% showed the weakest correlation (ρ=0.237) (FIG. 8A). Each of the cTF cohorts represented a variety of cancer types (FIG. 8B). Moreover, both the cTF≥10% and cTF=≥1% but lower than 10% showed high PPA, NPA and OPA values as compared to the overall cohort for both 10 and 14 mutations/Mb bTMB thresholds (Tables 3-5).

TABLE 3
All pairs

| N = | bTMB | bTMB |
|---|---|---|
| 597 | 10 | 14 |
| PPA | 55% | 36% |
| NPA | 94% | 99% |
| OPA | 86% | 87% |

TABLE 4
Pairs w/cTF ≥1%

| N = | bTMB | bTMB |
|---|---|---|
| 447 | 10 | 14 |
| PPA | 73% | 48% |
| NPA | 92% | 98% |
| OPA | 88% | 89% |

TABLE 5
Pairs with cTF ≥10%

| N = | bTMB | bTMB |
|---|---|---|
| 170 | 10 | 14 |
| PPA | 87% | 58% |
| NPA | 86% | 95% |
| OPA | 86% | 87% |

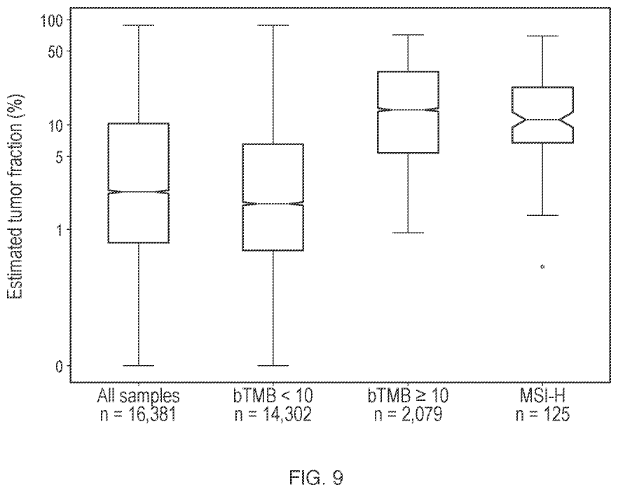

A second cohort of 16,381 liquid biopsy results was analyzed to determine the distribution of tumor shed for bTMB<10 mutations/Mb, bTMB≥10 mutations/Mb and microsatellite instability-high (MSI-H) status (FIG. 9). The distribution of samples with bTMB≥10 mutations/Mb and MSI-H status was skewed towards tumor fractions higher than 1%, as compared to all samples and samples with bTMB<10 mutations/Mb. These results indicate that determination of TMB and MSI status would be more accurate for samples with cTF≥1%.

### Conclusions

Complex biomarkers, such as TMB and MSI status, in liquid biopsy samples is better correlated with tumor biopsy results when cTF≥1%.

### Example 5: Association of Tumor Shed with Homologous Repair Deficiency Score in Liquid Biopsy Samples

This example describes the investigation of the effect of tumor fraction on the detection of homologous repair deficiency (HRD) score in liquid biopsy samples from cancer patients.

### Materials and Methods

### Effect of Tumor Shed on HRD Score Determination

A pan-cancer cohort patient—with available paired liquid and tissue biopsy results were analyzed. Tumor shed was calculated by cTF, and the samples were stratified as cTF>10%, cTF between 1% and 10%, and cTF<1% (including samples with cTF=0%). HRD scores for each sample were then determined.

For HRD score determination, a pan-cancer genomic profiling dataset (n=202,472) was split 70:30 for training and validation of an HRD signature using an XGBoost machine learning model (mlHRD). A broad set of copy number (Macintyre, G., et al. “(2018). Nature genetics 50 (9): 1262-1270.), as well as indel features (Alexandrov, L. B., et al. (2020). Nature 578 (7793): 94-101) were used to identify signatures of HRD. Scores were also applied to a set of validation samples.

### Results

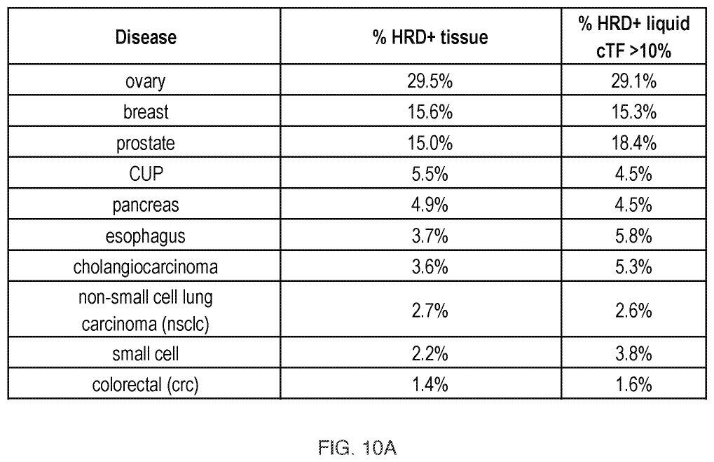

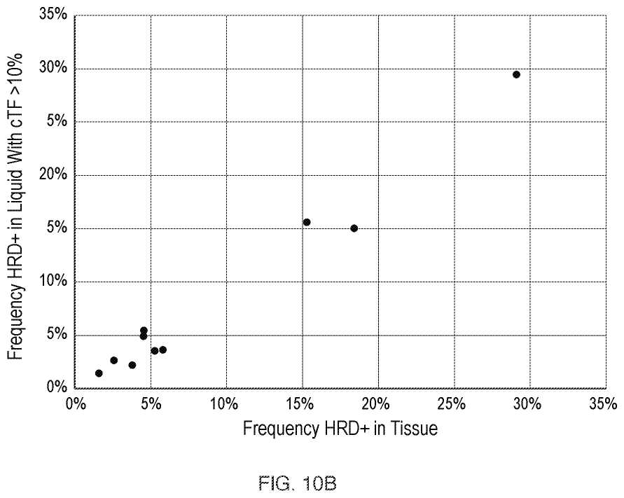

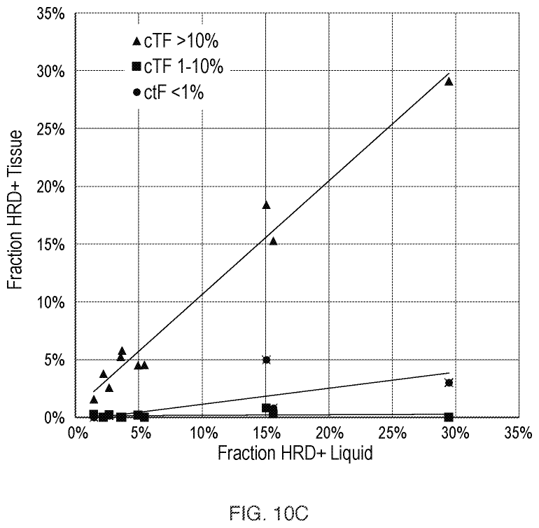

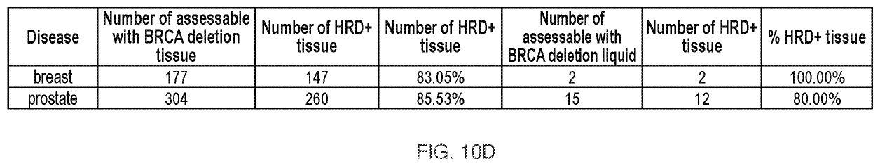

HRD score detection rates were lower in samples with cTF of <1% and 1-10% ctDNA (Table8). Higher detection rates of HRD scores were observed for samples with cTF>10% (Table 6 and FIGS. 10A-10D).

### Conclusions

Detection of HRD scores in liquid biopsy samples is better correlated with tumor biopsy results when cTF>10%.

TABLE 6

| | Total | | | | No. of | % | | No. of | % | | No. of | % |
|---|---|---|---|---|---|---|---|---|---|---|---|---|
| | no. of | No. of | % | No. of | HRD + | HRD + | No. of | HRD + | HRD + | No. of | HRD + | HRD + |
| | assessable | HRD + | HRD + | assessable | liquid | liquid | assessable | liquid | liquid | assessable | liquid | liquid |
| | tissue | tissue | tissue | cTF < | cTF < | cTF < | cTF | cTF | cTF | cTF > | cTF > | cTF > |
| Disease | samples | samples | samples | 1% | 1% | 1% | 1-10% | 1-10% | 1-10% | 10% | 10% | 10% |
| ovary | 13083 | 3855 | 29.47% | 91 | 0 | 0.00% | 166 | 5 | 3.01% | 55 | 16 | 29.09% |
| breast | 22632 | 3533 | 15.61% | 671 | 2 | 0.30% | 861 | 7 | 0.81% | 583 | 89 | 15.27% |
| prostate | 11084 | 1667 | 15.04% | 1007 | 8 | 0.79% | 1501 | 75 | 5.00% | 995 | 183 | 18.39% |
| unknown | 9955 | 543 | 5.45% | 202 | 0 | 0.00% | 297 | 0 | 0.00% | 220 | 10 | 4.55% |
| primary | | | | | | | | | | | | |
| carcinoma | | | | | | | | | | | | |
| (cup) | | | | | | | | | | | | |
| pancreas | 14445 | 712 | 4.93% | 550 | 1 | 0.18% | 469 | 1 | 0.21% | 133 | 6 | 4.51% |
| gallbladder | 1441 | 64 | 4.44% | 23 | 0 | 0.00% | 30 | 0 | 0.00% | 19 | 1 | 5.26% |
| endometrial | 7084 | 303 | 4.28% | 49 | 0 | 0.00% | 67 | 0 | 0.00% | 19 | 1 | 5.26% |
| stomach | 3832 | 160 | 4.18% | 55 | 0 | 0.00% | 51 | 1 | 1.96% | 38 | 3 | 7.89% |
| esophagus | 6474 | 237 | 3.66% | 58 | 0 | 0.00% | 107 | 0 | 0.00% | 69 | 4 | 5.80% |
| cholangio | 4685 | 167 | 3.56% | 143 | 0 | 0.00% | 137 | 0 | 0.00% | 76 | 4 | 5.26% |
| carcinoma | | | | | | | | | | | | |
| liver | 1006 | 29 | 2.88% | 11 | 0 | 0.00% | 21 | 0 | 0.00% | 23 | 0 | 0.00% |
| unknown | 1328 | 38 | 2.86% | 14 | 0 | 0.00% | 14 | 0 | 0.00% | 24 | 0 | 0.00% |
| primary- | | | | | | | | | | | | |
| neuro | | | | | | | | | | | | |
| lung non- | 40961 | 1086 | 2.65% | 1182 | 3 | 0.25% | 2044 | 3 | 0.15% | 778 | 20 | 2.57% |
| small cell | | | | | | | | | | | | |
| lung | | | | | | | | | | | | |
| carcinoma | | | | | | | | | | | | |
| (nsclc) | | | | | | | | | | | | |
| bladder | 5493 | 142 | 2.59% | 29 | 0 | 0.00% | 78 | 1 | 1.28% | 47 | 8 | 17.02% |
| soft tissue | 484 | 11 | 2.27% | 36 | 0 | 0.00% | 31 | 0 | 0.00% | 11 | 0 | 0.00% |
| sarcoma | | | | | | | | | | | | |
| endocrine- | 862 | 19 | 2.20% | 33 | 0 | 0.00% | 17 | 0 | 0.00% | 27 | 0 | 0.00% |
| neuro | | | | | | | | | | | | |
| small cell | 2502 | 55 | 2.20% | 8 | 0 | 0.00% | 24 | 0 | 0.00% | 53 | 2 | 3.77% |
| head and | 3736 | 71 | 1.90% | 46 | 0 | 0.00% | 64 | 0 | 0.00% | 23 | 0 | 0.00% |
| neck | | | | | | | | | | | | |
| melanoma | 6263 | 117 | 1.87% | 48 | 0 | 0.00% | 66 | 0 | 0.00% | 41 | 1 | 2.44% |
| colorectal | 27359 | 393 | 1.44% | 353 | 1 | 0.28% | 443 | 0 | 0.00% | 443 | 7 | 1.58% |
| (crc) | | | | | | | | | | | | |
| kidney | 3595 | 49 | 1.36% | 62 | 0 | 0.00% | 77 | 1 | 1.30% | 26 | 1 | 3.85% |
| thyroid | 1799 | 8 | 0.44% | 32 | 0 | 0.00% | 42 | 0 | 0.00% | 7 | 0 | 0.00% |

1. (canceled)

2. A method of treating an individual having a cancer with an immuno-oncology (IO) therapy and chemotherapy combination comprising (a) determining a tumor shed value for a liquid biopsy sample obtained from the individual, and (b) treating the individual with the IO therapy and chemotherapy combination if the tumor shed value in the liquid biopsy sample is equal to or higher than a reference tumor shed value.

3-7. (canceled)

8. A method of treating an individual having a cancer with an immuno-oncology (IO) therapy comprising (a) determining a tumor shed value for a liquid biopsy sample obtained from the individual, and (b) treating the individual with the IO if the tumor shed value in the liquid biopsy sample is lower than a reference tumor shed value.

9-14. (canceled)

15. A method of treating an individual having a cancer with a first therapy and a second therapy comprising (a) determining a tumor shed value for a liquid biopsy sample obtained from the individual, and (b) treating the individual with the first therapy and the second therapy if the tumor shed value in the liquid biopsy sample is equal to or greater than a reference tumor shed value.

16-40. (canceled)

41. The method of claim 2, wherein the tumor shed value is determined by composite tumor fraction (cTF) or by a tumor fraction estimator (TFE) process.

42. The method of claim 41, wherein the tumor shed value is determined by cTF using a method comprising:
(a) receiving a plurality of values, each value indicative of an allele fraction at a corresponding locus within a subgenomic interval in the sample;
(b) determining a certainty metric value indicative of a dispersion of the plurality of values;
(c) determining a first estimate of the tumor fraction of the sample, the first estimate based on the certainty metric value for the sample and a predetermined relationship between one or more stored certainty metric values and one or more stored tumor fraction values;
(d) determining whether a value associated with the first estimate is greater than a first threshold, wherein if the value associated with the first estimate is greater than the first threshold, the first estimate determined as the tumor fraction of the sample; and
(e) if the value associated with the first estimate is less than or equal to the first threshold, determining a second estimate of the tumor fraction of the sample based on an allele frequency determination, wherein the second estimate is determined as the tumor fraction of the sample.

43. The method of claim 42, wherein:
(a) the tumor fraction is a value indicative of a ratio of circulating tumor DNA (ctDNA) to total cell-free DNA (cfDNA) in the sample;
(b) the first threshold is indicative of a minimum detectable quantity for the tumor fraction of the sample;
(c) determining whether the value associated with the first estimate of the tumor fraction of the sample is greater than the first threshold comprises determining whether the first estimate is greater than a defined tumor fraction threshold; and/or
(d) determining whether the value associated with the first estimate of the tumor fraction of the sample is greater than a first threshold comprises determining whether a statistical lower bound associated with the first estimate is greater than 0.

44-46. (canceled)

47. The method of claim 42, wherein determining the second estimate of the tumor fraction of the sample based on the allele frequency determination comprises:
(a) determining whether a quality metric for the plurality of values is greater than a second threshold;
(b) based on a determination that the quality metric for the plurality of values is greater than the second threshold, determining the second estimate for the tumor fraction of the sample based on a first determination of somatic allele frequency, and
(c) based on a determination that the quality metric for the plurality of values is less than or equal to the second threshold, determining the second estimate for the tumor fraction of the sample based on a second determination of somatic allele frequency.

48. The method of claim 47, wherein:
(a) the quality metric for the plurality of values is indicative of an average sequence coverage for the sample, an allele coverage at each loci corresponding to the plurality of values, a degree of nucleic acid contamination in the sample, a number of single nucleotide polymorphism (SNP) loci within the loci corresponding to the plurality of values, or any combination thereof;
(b) the second threshold comprises a specified lower limit of the quality metric;
(c) the first determination of somatic allele frequency comprises a determination of variant allele frequencies associated with the plurality of values after excluding variant alleles that are present at an allele frequency greater than an upper bound for the first estimate of the tumor fraction of the sample, and the second estimate of the tumor fraction of the sample is set equal to a maximum variant allele frequency detected;
(d) the second determination of somatic allele frequency comprises a determination of variant allele frequencies for all variant alleles associated with the plurality of values, and the second estimate of the tumor fraction of the sample is set equal to a maximum variant allele frequency detected;
(e) the first determination of somatic allele frequency and the second determination of somatic allele frequency further comprise removing variant allele frequencies from the determination that correspond to germline variants, clonal hematopoiesis of indeterminate potential (CHIP) variants, and sequencing artifact variants, prior to determining the second estimate of the tumor fraction of the sample;
(f) the first determination of somatic allele frequency and the second determination of somatic allele frequency further comprise removing variant allele frequencies that correspond to variants of unknown significance prior to determining the second estimate of the tumor fraction of the sample;
(g) each value within the plurality of values is an allele fraction;
(h) each value within the plurality of values comprises a ratio of the difference in abundance between a maternal allele and a paternal allele relative to abundance of the maternal allele or the paternal allele at the corresponding locus; and/or
(i) the certainty metric value for the sample is indicative of a deviation of each of the plurality of values from a corresponding expected value.

49-65. (canceled)

66. The method of claim 47, further comprising determining a probability distribution function for the plurality of values; wherein the certainty metric value for the sample is determined using the probability distribution function.

67. (canceled)

68. The method of claim 47, wherein;
(a) the corresponding loci comprise one or more loci having a different maternal allele and paternal allele;
(b) the corresponding loci consist of loci having a different maternal allele and paternal allele; or
(c) the corresponding loci comprise one or more loci having the same maternal allele and paternal allele.

69-70. (canceled)

71. The method of claim 41, wherein the tumor shed value is determined by a TFE process using a method comprising:
(a) receiving a plurality of values, each value indicative of an allele fraction at a corresponding locus within a subgenomic interval in the sample;
(b) determining a certainty metric value indicative of a dispersion of the plurality of values; and
(c) determining an estimate of the tumor fraction of the sample based on the certainty metric value for the sample and a predetermined relationship between one or more stored certainty metric values and one or more stored tumor fraction values, wherein the estimate is determined as the tumor fraction of the sample.

72. The method of claim 71, wherein:
(a) the tumor fraction is a value indicative of a ratio of ctDNA to total cfDNA in the sample;
(b) each value within the plurality of values is an allele fraction;
(c) each value within the plurality of values comprises a ratio of the difference in abundance between a maternal allele and a paternal allele relative to abundance of the maternal allele or the paternal allele at the corresponding locus;
(d) the certainty metric value for the sample is indicative of a deviation of each of the plurality of values from a corresponding expected value;
(e) the plurality of values comprises a plurality of allele coverages;
(f) the method further comprises determining a probability distribution function for the plurality of values; wherein the certainty metric value for the sample is determined using the probability distribution function; and/or
(g) the corresponding loci comprise one or more loci having a different maternal allele and paternal allele, consist of loci having a different maternal allele and paternal allele, or comprise one or more loci having the same maternal allele and paternal allele.

73-81. (canceled)

82. The method of claim 2, wherein the reference tumor shed value is between 0.5% to 10.0%.

83-87. (canceled)

88. The method of claim 2, wherein the IO therapy comprises a single IO agent or multiple IO agents.

89. The method of claim 2, wherein the IO therapy comprises an immune checkpoint inhibitor.

90-98. (canceled)

99. The method of claim 2, wherein the chemotherapy comprises a single chemotherapeutic agent or multiple therapeutic agents.

100-121. (canceled)

122. The method of claim 2, wherein the liquid biopsy sample comprises blood, plasma, serum, cerebrospinal fluid, sputum, stool, urine, or saliva and comprises mRNA, DNA, circulating tumor DNA (ctDNA), cell-free DNA, or cell-free RNA from the cancer.

123-124. (canceled)

125. The method of claim 2, wherein the tumor shed value is determined by sequencing.

126-132. (canceled)

133. The method of claim 2, wherein the cancer is a B cell cancer, a melanoma, breast cancer, lung cancer, bronchus cancer, colorectal cancer or carcinoma, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer or carcinoma, lung non-small cell lung carcinoma (NSCLC), head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

134. (canceled)

135. The method of claim 2, wherein the individual is a human.

136-137. (canceled)
